(19) United States
(12) Patent Application Publication
(10) Pub. No.: US 2024/0309418 A1
(43) Pub. Date: Sep. 19, 2024

(54) ENZYMATIC METHODS AND COMPOSITIONS FOR POLYMERIZATION OF PHOSPHORAMIDATE AND THIOPHOSPHORAMIDATE-LINKED DNA

(71) Applicant: The General Hospital Corporation, Boston, MA (US)

(72) Inventors: Victor Lelyveld, Lexington, MA (US); Jack Szostak, Boston, MA (US)

(21) Appl. No.: 18/605,332

(22) Filed: Mar. 14, 2024

Related U.S. Application Data
(60) Provisional application No. 63/490,022, filed on Mar. 14, 2023.

(51) Int. Cl. C12P 19/34; C12N 9/12

(52) U.S. Cl. CPC C12P 19/34; C12N 9/1252; C12Y 207/07007

(57) ABSTRACT

Provided herein, in some embodiments, are improved enzymatic methods for producing an oligonucleotide or a polynucleotide comprising phosphoramidate-linked nucleotides or thiophosphoramidate-linked nucleotides, and compositions comprising the oligonucleotide or the polynucleotide thus produced.

### CLAIM OF PRIORITY

This application claims the benefit of U.S. Provisional Patent Application No. 63/490,022, filed Mar. 14, 2023, which is incorporated by reference herein in its entirety.

### SEQUENCE LISTING

This application contains a Sequence Listing that has been submitted electronically as an XML file named 29539-0734001_SL_ST26.xml. The XML file, created on Mar. 12, 2024, is 10,910 bytes in size. The material in the XML file is hereby incorporated by reference in its entirety.

### FIELD OF THE INVENTION

The subject matter disclosed herein generally relates to oligonucleotides and polynucleotides comprising N3′→P5′ phosphoramidate (NP) and thiophosphoramidate (NPS) bonds, and enzymatic methods and compositions for producing such.

### BACKGROUND OF THE INVENTION

DNA polymerases copy genetic material by catalyzing phosphodiester bond formation. This highly conserved activity proceeds by a common mechanism, such that incorporated nucleoside analogs terminate chain elongation if the resulting primer strand lacks a terminal hydroxyl group. Even conservatively substituted 3′-amino nucleotides generally act as chain terminators, and no enzymatic pathway for their polymerization has yet been found. Although 3′-amino nucleotides can be chemically coupled to yield stable oligonucleotides or polynucleotides containing N3′→P5′ phosphoramidate (NP) bonds, no such internucleotide linkages are known to be enzymatically synthesized and to occur in nature.

### SUMMARY OF THE INVENTION

The present disclosure is based, at least in part, on the surprising discovery that 3′-amino terminated primers can be extended by DNA polymerase from B. stearothermophilus in a template-directed manner to produce oligonucleotides or polynucleotides comprising N3′→P5′ phosphoramidate (NP) bonds. It has also been demonstrated that faster rates of extension can be achieved with Sc³⁺ among trivalent metal ions or Ca²⁺ among divalent metal ions or other metal ion cofactors and/or with DNA polymerase comprising a single active site mutation. It has also been demonstrated that a nonbridging thio-substituted N3′→P5′ thiophosphoramidate bond can be formed in a similar manner.

Accordingly, aspects of the present disclosure provide a method for producing an oligonucleotide or a polynucleotide comprising phosphoramidate-linked nucleotides, the method comprising incubating a sample comprising a DNA polymerase variant comprising an amino acid sequence that is at least 70% identical to SEQ ID NO: 1, wherein the amino acid sequence comprises an amino acid substitution at position F710, a divalent and/or trivalent metal ion cofactor, 3′-amino-2′,3′-dideoxyribonucleoside 5′-triphosphates (nNTPs), a 3′-amino terminated primer, and a DNA template comprising, from 3′ to 5′, a sequence complementary to the primer and a nucleic acid sequence of interest, under conditions and for a time sufficient for the DNA polymerase variant to produce the oligonucleotide or the polynucleotide comprising phosphoramidate-linked nucleotides. In another aspect of the disclosure, the sample can include one or more deoxyribonucleoside 5′-(α-P-thio)triphosphates (dNTPαS) substrates to produce an oligonucleotide or a polynucleotide containing one or more N3′→P5′ thiophosphoramidate bonds incorporating a nonbridging sulfur substitution.

In some embodiments, the amino acid sequence is at least 80% identical to SEQ ID NO: 1. In some embodiments, the amino acid sequence is at least 90% identical to SEQ ID NO: 1. In some embodiments, the amino acid sequence is at least 95% identical to SEQ ID NO: 1. In some embodiments, the amino acid substitution at position F710 is F710Y. In some embodiments, the DNA polymerase variant comprises the amino acid sequence set forth in SEQ ID NO: 1.

In some embodiment, the DNA polymerase variant is prepared from cells that express the DNA polymerase variant. In some embodiments, the DNA polymerase variant is in a cell lysate from cells that express the DNA polymerase variant.

In some embodiments, the sample is incubated at a temperature of 50 to 65° C. In some embodiments, the sample is incubated at a temperature of 55° C.

In some embodiments, the sample is incubated for 1 to 24 hours. In some embodiments, the sample is incubated for 24 hours.

In some embodiments, the sample is incubated at a pH of 7 to 10. In some embodiments, the sample is incubated at a pH of 8.6-9.2. In some embodiments, the sample is incubated at a pH of 8.8.

In some embodiments, a divalent metal ion cofactor is selected from the group consisting of Ba²⁺, Sr²⁺, Ca²⁺, Mg²⁺, Mn²⁺, Co²⁺, and Zn²⁺. In some embodiments, the divalent metal ion cofactor is Ca²⁺.

In some embodiments, a trivalent metal ion cofactor is selected from the group consisting of Sc³⁺, Lu³⁺, In³⁺, Y³⁺, Eu³⁺, Gd³⁺, Yb³⁺, or Er³⁺. In some embodiments, the trivalent metal ion is Sc³⁺.

In some embodiments, the substrate mixture comprises nATP, nGTP, nCTP, and/or nTTP.

In some embodiments, the substrate mixture comprises dATPαS, dGTPαS, dCTPαS, and/or dTTPαS.

In some embodiments, a polyamine or its respective salt is selected from the group consisting of spermine, putrescine, spermidine, cadaverine, thermospermine, caldopentamine, and caldohexamine. In some embodiments, the polyamine is spermine tetrahydrochloride.

In some embodiments, the 3′-amino terminated primer comprises ribonucleotides and/or deoxyribonucleotides.

In some embodiments, the 3′-amino terminated primer comprises a 3′-amino terminal ribonucleotide selected from the group consisting of 3′-amino-adenosine, 3′-amino-guanosine, 3′-amino-cytidine, and 3′-amino-uridine.

In some embodiments, the 3′-amino terminated primer comprises a 3′-amino terminal dideoxynucleotide selected from the group consisting of 3′-amino-2′,3′-dideoxyadenosine (nA), 3′-amino-2′,3′-dideoxythymidine (nT), 3′-amino-2′,3′-dideoxycytidine (nC), or 3′-amino-2′,3′-dideoxyguanosine (nG).

In some embodiments, the 3′-amino terminated primer comprises a label.

In some embodiments, the 3′-amino terminated primer comprises phosphodiester-linked nucleotides and/or phosphoramidate-linked nucleotides.

In some embodiments, the 3′-amino terminated primer is 5 to 200 nucleotides in length.

In some embodiments, the 3′-amino terminated primer comprises at least 5 consecutive phosphoramidate-linked nucleotides. In some embodiments, the 3′-amino terminated primer comprises at least 25 consecutive phosphoramidate-linked nucleotides.

In some embodiments, each nucleotide in the 3′-amino terminated primer is phosphoramidate-linked.

In some embodiments, the oligonucleotide or the polynucleotide is 25 to 250 nucleotides in length.

In some embodiments, the oligonucleotide or the polynucleotide comprises phosphoramidate-linked nucleotides and phosphodiester-linked nucleotides.

In some embodiments, the oligonucleotide or the polynucleotide comprises at least 25 consecutive phosphoramidate-linked nucleotides. In some embodiments, the oligonucleotide or the polynucleotide comprises at least 50 consecutive phosphoramidate-linked nucleotides. In some embodiments, the oligonucleotide or the polynucleotide comprises at least 100 consecutive phosphoramidate-linked nucleotides. In some embodiments, each nucleotide in the oligonucleotide or the polynucleotide is phosphoramidate-linked.

In some embodiments, the sample further comprises nucleoside triphosphates (NTPs). In some embodiments, the NTPs comprise deoxynucleoside triphosphates (dNTPs).

Aspects of the present disclosure provide an oligonucleotide or a polynucleotide comprising a plurality of phosphoramidate linkages, wherein the oligonucleotide or the polynucleotide is 25 to 250 nucleotides in length.

In some embodiments, the oligonucleotide or the polynucleotide is 25 to 150 nucleotides in length. In some embodiments, the oligonucleotide or the polynucleotide is 25 to 100 nucleotides in length.

In some embodiments, the oligonucleotide or the polynucleotide comprises phosphoramidate-linked nucleotides and phosphodiester-linked nucleotides.

In some embodiments, the oligonucleotide or the polynucleotide comprises at least 25 consecutive phosphoramidate-linked nucleotides. In some embodiments, the oligonucleotide or the polynucleotide comprises at least 50 consecutive phosphoramidate-linked nucleotides. In some embodiments, the oligonucleotide or the polynucleotide comprises at least 100 consecutive phosphoramidate-linked nucleotides. In some embodiments, each nucleotide in the oligonucleotide or the polynucleotide is phosphoramidate-linked.

In some embodiments, the oligonucleotide or the polynucleotide comprises ribonucleotides and/or deoxyribonucleotides.

### BRIEF DESCRIPTION OF THE DRAWINGS

FIGS. 1A-1F show reactant and product polymerase “open” complexes containing a 3′-amino primer or a N3′→P5′ phosphoramidate bond. FIG. 1A shows a cartoon of ternary complexes. The “0 complex” contains wild-type DNA polymerase I (BF) from the thermophilic bacterium Bacillus stearothermophilus, 3′-amino terminal DNA primer (5′-GCGATCAGnC (SEQ ID NO:2), black), and DNA template (5′-ACACGCTGATCGCA (SEQ ID NO:3), gray) in an open conformation. The “+1 complex” formed by in-situ primer extension with dGTP, forming a phosphoramidate linkage, followed by translocation. FIG. 1B shows the 0 complex structure solved at 2.25 Å. BF is ribbon cartooned, bound to unextended primer/template in the open conformation (PDB 6UR4). FIG. 1C shows an overlay of the 3′-terminal nC sugar in the 0 complex (gray bonds) and that of a dC terminated primer (gray, PDB IL5U) (Johnson et al., Processive DNA synthesis observed in a polymerase crystal suggests a mechanism for the prevention of frameshift mutations. Proc. Natl. Acad. Sci. 100, 3895-3900 (2003)), as well as the 2Fo−Fc density map (gray mesh, 2 σ) associated with the terminal nC:dG base pair. FIG. 1D shows a structure and omit map (top, gray mesh, 4 σ) for the terminal dG (+1) residue extended in situ from the 3′-nC (0) terminated primer in the +1 complex and chemical structure as refined (bottom). FIG. 1E shows the +1 complex structure solved at 2.27 Å in an open post-translocated conformation with +1 extended primer and template (PDB 6UR2). FIG. 1F shows an enlarged view of the +1 complex active site, showing the extended dG residue and associated 2Fo−Fc map overlay (gray mesh, 2 σ).

FIGS. 2A-2D show polymerase-catalyzed 3′-amino primer extension and NP-DNA synthesis. FIG. 2A shows a graph of the effect of divalent metal ion cofactors at 5 mM with 1 μM 3′-amino terminal DNA primer, 1.5 μM DNA template, and 1.1 μM wild-type BF at 55° C. in 40 mM Tris-HCl, pH 8.8, and 1 mM DTT (omitted for Co²⁺). Error bars are s.e.m. for n=2. FIG. 2B shows a graph of the observed rate constant, k_{obs}, of 3′-amino primer extension by wild-type BF in the presence of 1 mM dCTP and varying concentrations of CaCl₂ (gray, kₚₒₗ=0.075 min⁻¹ and K_{d,app}=2.1 mM) or MgCl₂ (black, kₚₒₗ=0.015 min⁻¹ and K_{d,app}=1.3 mM). FIG. 2C shows a time course of 3′-amino primer extension reactions (right) with 1 mM dCTP and 10 mM CaCl₂ or MgCl₂ visualized by denaturing PAGE. Top: fluorescein-labeled 3′-amino terminal primer (GCGTCCATTCGTTACCTG-NH₂; SEQ ID NO: 4) and template DNA duplex (CGCAGGTAAGCAATGGACGGACT; SEQ ID NO: 5) for FIGS. 2A-2C. FIG. 2D shows the chemical structure of nNTP substrates (left), and time course of 3′-amino primer extension reactions with 1 mM nNTPs and 10 mM CaCl₂ (right). Top: Fluorescein-labeled 3′-amino terminal primer (GCGTCCATTCGTTACCTG-NH₂; SEQ ID NO: 4) and template DNA duplex with underlined region DNA or NP-DNA

(CGCAGGTAA [symbol] ACGCACTCATAAGTTGTAAAGGC;
SEQ ID NO: 6)

FIGS. 3A-3H show discrimination between dNTP and nNTP substrates. FIG. 3A shows a time course of 3′-amino primer extension with either 1 mM dCTP or nCTP with wild-type BF and conditions as in FIGS. 2A-2D. FIG. 3B shows a graph of substrate-dependent rate constants for extension of a 3′-amino primer with varying concentrations of dCTP (black) or nCTP (gray) with 10 mM CaCl₂ under pre-steady-state conditions. Parameter estimates in Table 1. FIG. 3C shows a cartoon of BF closed complex with DNA primer/template and bound substrate (PDB 3EZ5) (Golosov et al., The Mechanism of the Translocation Step in DNA Replication by DNA Polymerase I: A Computer Simulation Analysis. Structure 18, 83-93 (2010)) with mutated active site residues indicated. FIG. 3D shows a graph of observed rates of 3′-amino primer extension with either 1 mM dCTP (black) or nCTP (gray) for wild-type (WT) BF and various point mutants. FIG. 3E shows a graph of substrate-dependent rate constants, as in FIG. 3B, for the mutant F710Y (see Table 4). FIG. 3F shows data from a 3′-amino primer extension by mutant F710Y on a mixed sequence template (sequence in FIG. 2D) with either 1 mM nNTPs or dNTPs at 55° C. in a buffer containing 40 mM Tris-HCl, pH 8.8, 10 mM CaCl₂, and 1 mM DTT. FIG. 3G shows data from extension reactions as in FIG. 3F with nNTP drop-out mixes (left: 0.5 mM each nNTP or only three of four, as indicated) or dNTP drop-in mixes (right: 0.5 mM each dNTP or only three of four, as indicated, with fourth complemented by with the corresponding nNTP). The gel shows reactions incubated for either 7 hr (lanes 2-6) with nNTP drop-out mixes or 10 min (lanes 8-12) with dNTP drop-in mixes. “P” indicates lanes containing primer alone. FIG. 3H shows data from a 3′-amino primer extension reactions with F710Y as in FIG. 3F, but showing acid digest products after 24 hr of extension (HOAc lanes).

FIGS. 4A-4D shown structural snapshots of the reaction pathway in NP catalysis. FIG. 4A show the reactant “0 complex” in the open conformation, with bound DNA duplex containing a 3′-amino terminal primer aligned at the active site, as in FIGS. 1B-1C (PDB 6UR4). FIG. 4B shows the closed conformation reaction complex bound to DNA duplex containing a 3′-amino terminal primer, substrate analog dGpNHpp, and Mn²⁺ (PDB 6US5). FIG. 4C shows the closed conformation reaction complex bound to DNA duplex containing a 3′-dideoxy terminal primer, as well as nGTP substrate and Ca²⁺ (6UR9). FIG. 4D shows the post-translocated product “+1 complex” in the open conformation, containing an incorporated N3′→P5′ bond between the primer terminal nC and dG residues, as in FIGS. 1D-1F (PDB 6UR2). Atomic distances are indicated in Å, and the orientation of the O helix in each structure is shown with a gray arrow.

FIGS. 5A-5B show structural model for the N—P bond forming reaction in BF. FIG. 5A shows the structure and Fo−Fc omit map (top, gray mesh, 5 σ) for the observed closed reaction complex bound to DNA duplex containing a ddC terminal primer, nGTP substrate, and Ca²⁺. FIG. 5B shows an early model of the calcium-bound reaction complex with attacking 3′-amino group (black) of the primer terminus (N) in reaction with the α-phosphate center (gray) of an incoming substrate (N₊₁) and a coordinated metal ion (M²⁺, gray). Interacting residues found in closed complex structures are indicated.

FIG. 6 shows data from 3′-amino terminal primer extension under crystallization conditions. Reactions were carried out with 1 μM of the indicated primer and 1.5 μM of the indicated template at 37° C. in 0.2 M sodium succinate, pH 7.0, with 10 mM dGTP and 10 mM DTT. Samples quenched 1:25 in 90% formamide with 10 mM EDTA were separated on a 24% denaturing polyacrylamide TBE-urea gel and visualized with a laser scanning fluorescence imager. TS, Thermoscript reverse transcriptase (Life Technologies); HIV1, HIV-1 reverse transcriptase (Worthington Biochemical); EcK, E. coli Klenow fragment
(exo-, New England Biolabs); Bst, Bst DNA polymerase large fragment 3.0 variant (New England Biolabs).

FIGS. 7A-7B show high resolution mass spectrometry detection of NP-bonded primer extension products. FIG. 7A shows primer extension with the indicated primer (2 μM) and template (2.2 μM) at 37° C. in 0.2 M sodium succinate, pH 7.0, with 10 mM dGTP, 10 mM DTT, and 10 mM MgCl₂ where indicated. BF, wild-type Bst DNA polymerase large fragment. FIG. 7B shows high resolution mass spectra of extended NP-bonded extended products observed as the [M-6H]⁶⁻ ions detected in samples of BF reactions with (gray, top) or without (gray, bottom) the addition of Mg²⁺, prepared from the same samples as in lanes indicated in FIG. 7A with dashed boxes of the corresponding color. Predicted isotope distribution for the indicated chemical formulas overlaid in red boxes.

FIGS. 8A-8D show that N3′→P5′ NP polymerase activity proceeds in crystallo by a one-metal-ion mechanism. FIG. 8A shows condensation of 3′-amino 2′,3′-dideoxynucleoside 5′-triphosphates (nNTPs) generates N3′→P5′ phosphoramidate DNA. FIG. 8B shows reaction scheme for BF-catalyzed primer extension of a 3′-amino terminal primer on a DNA template, yielding pyrophosphate. R=OH for in crystallo single nucleotide addition of dGTP with co-crystallized divalent metal ion cofactor Ca²⁺, whereas R=NH₂ for multiple turnover extension in solution to synthesize N3′→P5′ NP-DNA polynucleotide strands in the presence of divalent (M²⁺) or trivalent (M³⁺) metal ion cofactors. FIG. 8C shows ribbon cartoons of BF polymerase ground state (GS) holocomplex X-ray crystal structure with 3′-amino terminal DNA primer (gray ribbon) and DNA template (light gray ribbon) with bound dGTP substrate and a single Ca²⁺ ion (gray atom) in the canonical metal-B site (left and middle panels), showing one of two complexes co-crystallized in the asymmetric unit. All analyses performed here derive from the first complex (chains A, B, and C), but consistent trends are seen in both. Dotted circle indicates the position of a missing A-site metal in analogous models of the canonical phosphodiester synthesis mechanism but not observed in closed conformation GS structures containing a 3′-amino terminal primer (middle panel) or product state (PS) structures of the in-situ synthesized N3′→P5′ phosphoramidate (NP) bond. FIG. 8D shows representative time-dependent difference maps (Fₒ−F_{c}) showing density changes in the BF F710Y/D598A active site along the reaction coordinate for NP bond formation in crystallo. GS holocomplex crystals containing a 3′-amino-2′,3′-dideoxycytidine (nC) terminated primer and DNA template were formed under acidic conditions (pH 5-6) in the presence of Ca²⁺ and dGTP, and the in situ reaction was initiated by transferring crystals from the mother liquor to a soak solution buffered at pH 8.8. The reaction in intact crystals was quenched at the indicated times by flash freezing for subsequent data collection. Positive (gray mesh) or negative (black mesh) isosurfaces are shown contoured at 3 σ, superimposed on the GS model. Bottom-left: Normalized density change extracted from difference maps at the nascent NP (gray; N-P) and scissile OP (black; P-O) bonds quantified from 19 individual crystals (dots), observed first-order rate constant estimates for the density changes (solid lines), and 95% confidence interval on the estimates (shaded area).

FIGS. 9A-9G show that one-metal-ion NP catalysis is limited by deficient substrate activation. FIG. 9A shows voxelwise linear regression analysis of density dynamics in crystallo. Difference map (Fₒ−F_{c}) densities derived from reacting crystals quenched at various times yield kinetic traces at a reference point (Ref.) or any query point of interest. A pairwise linear regression slope, β, of density changes at the reference vs. query points can be estimated for any two points in real space. FIG. 9B shows regression statistics of key density dynamics during N3′→P5′ synthesis in BF F710Y/D598A. Insets i to iv show difference map density derived from individual crystals at four major active-site blobs indicated in FIG. 9C. Left panels: first-order kinetics of density differences from individual crystals (dots) over reaction time, with values for the estimated rate constant±s.d. plotted (solid line±shaded area). Right panels: pairwise linear regressions, coefficients of determination (R²), and correlation coefficients (r) of difference map densities at the indicated site vs. the NP bond across all crystals. FIG. 9C shows pairwise linear regression “beta” map calculated at all points vs. the nascent NP bond (black wedge) with positive (gray mesh) or negative (black mesh) regression slopes, β, displayed as isosurfaces superimposed on the ground state structure, contoured at |β|>0.45. FIG. 9D shows Pearson correlation map of the active site contoured at IrI>0.9 calculated by pairwise comparison of all voxels vs. the peak of the nascent bond (black wedge), with positive (gray mesh) or negative (black mesh) correlation displayed as isosurfaces superimposed on the ground state structure. FIG. 9E shows alternative view of the beta map contoured at |β|>0.4, highlighting positive and negative beta peaks at sites (black wedges) associated with a conformational change at the side chain of Lys-706 (positive lobe quantified in FIG. 9B, lower left panel) in the direction of the substrate O_{α,62} bridging position and the scissile bond, as well as the conformation change in the dGTP substrate sugar (grey wedges, positive lobe quantified in FIG. 9B, upper right panel). FIG. 9F shows pairwise linear regression of difference map densities at the position of the missing A site metal vs. the nascent NP bond. FIG. 9G shows cartoon overlay of ground state (light gray) and product state (gray) models, with indicated conformational dynamics labeled as in FIGS. 9B-9C.

FIGS. 10A-10D show probing for rate-limiting chemistry in NP-DNA synthesis in solution. FIG. 10A shows pre-steady-state solvent deuterium kinetic isotope effect estimated from the slope of the line kₙ/k_{H2O}=1+n(q−1) for 3′-amino primer extension with 1 mM dCTP and 10 mM CaCl₂ in varying mole fractions, n, of D₂O. The quantity 1/φ is the SDKIE, estimated at 1.16 with 95% confidence interval (1.09, 1.23) in the shaded region. FIG. 10B shows atomic model distances in the ground state reaction complex for the primer-terminal (nC) 3′-amine to the D830 side chain or to the substrate P_{α}. Grey mesh overlay indicates 2Fₒ−F_{c} density map contoured at 2.5 σ. FIG. 10C shows effect of α-phosphorothioate substitution on pre-steady-state Ca²⁺-activated NP synthesis at 45° C. with 500 μM Sp-αS, Rp-αS, or unmodified dCTP substrates and BF F710Y. Inset: fluorescently-labeled primer and template strand for the experiments in panels A, C, and D. A representative 15% TBE-urea PAGE separation of quenched reaction samples is shown. Only the first addition to this primer forms an NP or NPS linkage in the presence of 2′-deoxyribose substrates, yielding subsequent phosphodiester or phosphorothioate products, including mismatch extension products. FIG. 10D shows elemental thio effects (labeled arrows, mean±s.d.) estimated from the pre-steady-state rate constants (kₚₒₗ, error bars indicate s.d. from duplicates), for reactions with the indicated substrates as in FIG. 10C.

FIGS. 11A-11C shows a comparison of reaction models for phosphodiester and phosphoramidate synthesis in BF. FIG. 11A shows a model for the canonical two-metal ion “di-divalent” mechanism for native phosphodiester activity. FIG. 11B shows a model for the mono-divalent mechanism for Ca²⁺ catalyzed NP activity. FIG. 11C shows a proposed mono-trivalent mechanism for rare earth catalyzed NP activity. Note that neither inner sphere waters nor exact proton transfer pathways have been depicted. Two rate-limiting proton transfers have been detected for native polymerase activity (12, 16), but proton transfer is not rate-limiting for mono-divalent NP activity with Ca²⁺. At least one proton must nevertheless be transferred to generate the phosphoramidate product from the attacking neutral amine. In BF, these transfers likely involve deprotonated D830 as a general base and protonated K706 as a general acid. (Change labels to include 3′-OH or 3′-NH2)

FIGS. 12A-12D shows that trivalent rare earth metal ion cofactors confer rapid NP polymerase activity. FIG. 12A shows pre-steady-state kₚₒₗ estimates for extension of a 3′-amino terminal DNA primer on a DNA template (cartoon at top) by BF F710Y/D598A at 55° C. in the presence of various 1:1 ammonium citrate-buffered trivalent metal cations at 5 mM in 40 mM Tris-HCl, pH 8.8, 2 mM βME, in reactions initiated by addition of 250 μM nCTP. Dotted lines indicate the level of activity with Ca²⁺. Inset: representative 15% TBE-urea PAGE of quenched reaction samples from BF F710Y/D598A 3′-amino primer extension reactions in the presence of 5 mM Sc³⁺ showing +1 and +2 NP-DNA extension at the indicated times. FIG. 12B shows mixed sequence NP-DNA synthesis with excess BF F710Y/D598A on a 71-nt (left image) or 100-nt (right image) DNA templating region at 55° C. from a 5′-fluorescein-labeled 3′-amino-terminal DNA primer in the presence of 1:1 ammonium citrate-buffered 5 mM Sc³⁺ or Lu³⁺, as indicated, in 40 mM Tris-HCl, pH 8.8, 10 mM βME, and 25 μM spermine-HCl. Reactions were initiated by addition of 250 μM of each nNTP, sampled and quenched at the indicated times, and samples were separated on 10% (left) or 8% (right) TBE-urea PAGE gels. FIG. 12C shows elemental thio effects (labeled arrows, mean±s.d.) for Sc³⁺-activated NP synthesis with 500 μM Sp-αS, Rp-αS, or unmodified dCTP substrates at 45° C. estimated from kₚₒₗ (error bars indicate s.d. from duplicates) using a fluorescently-labeled primer and template strand (cartoon at top), yielding exclusively +1 product due to high NP(S) specificity. FIG. 12D shows representative 15% TBE-urea PAGE separation of quenched samples for the reactions in FIG. 12C.

FIGS. 13A-13B show additional analysis maps of time-resolved NP extension in F710Y/D598A BF. FIG. 13A shows linear regression map against the nascent bond (black wedge). Isosurfaces shown contoured at |β|>0.9 and superimposed on the ground state model, as in FIGS. 9A-9G. FIG. 13B shows P-value map for time resolved NP primer extension in F710Y/D598A BF. The P-value map was calculated as (1−p) for the significance of pairwise correlation estimates in FIGS. 9A-9G, contoured at p<10⁻⁶ and presented as isosurfaces 20 superimposed on the ground state model.

FIGS. 14A-14E shows the structural distinctions between BF F710Y mutant and WT in the ground state (GS) and during NP bond formation in crystallo. FIG. 14A shows overlay of the active site in GS structures of F710Y and WT, both also incorporating the solvent-exposed crystal-contact mutation D598A, viewed from the perspective of the O-helix. Two ordered solvent molecules are apparent in the F710Y mutant, stabilized by the exocyclic hydroxyl of Y710 and the catalytic Ca²⁺ ion, with the resulting GS conformational distinctions indicated by black arrows. FIG. 14B shows time resolved progress of NP bond formation in wild-type BF. Crystals of wild-type BF protein (containing the crystal contact mutation D598A) were prepared and soaked essentially as in FIGS. 8A-8D in a mother liquor containing CaCl₂ and dGTP, except soaking was carried out to 48 h. Difference maps are shown contoured at 3 σ, superimposed on the GS model, but time points up to 8 h show insignificant progress at this contouring and are not shown. FIG. 14C shows voxel-wise linear regression analysis of density dynamics during N3′→P5′ synthesis in crystallo in wild-type (D598A) BF. Pairwise linear regression map is calculated at all voxels vs. the site of the nascent NP bond (labeled gray wedge) with positive (gray mesh) or negative (black mesh) regression slopes, β, displayed as isosurfaces superimposed on the GS structure model, contoured at |β|>0.45. Dashed arrows indicate conformational changes giving rise to the indicated high-beta lobes. FIG. 14D shows first-order kinetic regression of nascent NP bond in wild-type, as in FIGS. 8A-8D, derived from difference map densities arising from 20 crystals. FIG. 14E shows kinetic regression (left) and pairwise linear regression (right) for the nascent inner sphere water indicated in FIG. 14C (gray wedge).

FIGS. 15A-15B shows the determinants of trivalent cofactor activity. FIG. 15A shows multiple turnover extension in BF F710Y catalyzed by calcium chloride or various trivalent rare earth metal cofactors in the presence of stoichiometric citrate. Conditions for extension were the same as in FIG. 12B, except with 10 mM CaCl₂ in lanes 2-6, 5 mM ScCl₃ and 5 mM ammonium citrate in lanes 7-11, 5 mM LuCl₃ and 5 mM ammonium citrate in lanes 12-16, 5 mM InCl₃ and 5 mM ammonium citrate in lanes 17-21, and 4 mM YCl₃ and 4 mM ammonium citrate in lanes 22-26. FIG. 15B shows representative gel showing extension of a native DNA primer on a DNA template with nCTP and 5 mM Sc₃₊ or Lu₃₊ in the presence of stoichiometric citrate at 55° C. Conditions are otherwise as in FIG. 10B. Estimated pre-steady-state rate constants were 5.5±0.6×10⁻³ min⁻¹ and 8±1×10⁻³ for Lu³⁺, respectively, from independent duplicates (mean±s.d.).

FIGS. 16A-16D shows the effects of metal citrate concentration and polyamines on multiple turnover NP polymerase activity. FIG. 16A shows optimization of multiple turnover primer extension with BF F710Y protein and nNTP mix (250 μM each amino-nucleotide) and 5 mM ScCl₃ vs. various concentrations of ammonium citrate at 55° C. FIG. 16B shows multiple turnover primer extension, as in FIG. 16A, with a range of metal concentrations and fixed stoichiometry of Sc³⁺:citrate. FIG. 16C shows primer extension reactions as in FIG. 12B, except with spermine HCl at the indicated concentrations. FIG. 16D shows primer extension reactions as in FIG. 16C, but with the addition of putrescine HCl as the polyamine additive. The first lane of each gel is sampled from a fully assembled reaction master mix prior to addition of the indicated additive and nNTPs and therefore shows only unextended fluorescently-labeled primer.

FIGS. 17A-17B show the effects of Mg²⁺ and active site mutations on long NP-DNA primer extension activated by trivalent Sc³⁺. FIG. 17A shows primer extension reactions on the +71 template in the presence of 1:1 ScCl₃:ammonium citrate and the indicated concentrations 1:1 MgCl₂:ammonium citrate, at concentrations on the metal:citrate basis. Reaction samples were quenched at the indicated times, analyzed on a 15% TBE-urea gel, and otherwise carried out as in FIG. 12B. FIG. 17B shows primer extension reactions as in FIG. 12B, except with the indicated BF polymerase active site mutants. The enzyme variant labeled as F710Y additionally carries the neutral crystal-contact mutation D598A.

FIGS. 18A-18D shows high resolution mass spectrometry validation of Sc³⁺ extension products. Reactions carried out as described in FIG. 10, with 5 mM ScCl₃ and 5 mM ammonium citrate at pH 8.8. FIG. 18A shows extracted spectrum for a 3′-amino terminal DNA primer extended by two nC residues in a reaction containing nCTP substrate on a template with two G residues in the +1 and +2 sites, as in FIG. 12A. FIG. 18B shows extracted spectrum for a 3′-amino terminal DNA primer extended by a thiophosphoramidate (NPS) linkage in a reaction with Sp-dCTPαS, as in FIG. 12F, on a 3′-biotinylated version of the sample. FIG. 18C shows analysis of 3′-amino terminal DNA primer extended by the sequence n[CCTGA] on a 3′-biotinylated +5 template in the presence of all four nNTP substrates at 250 μM. FIG. 18D shows analysis of 3′-amino terminal DNA primer extended by the sequence n[CCTGATGCGTG] on a 3′-biotinylated +11 template in the presence of all four nNTP substrates at 250 μM. For all reactions, the extended single-stranded chimeric primer was isolated from the reaction as described in Materials and Methods. For each extracted clipped spectra the observed charge state envelope is shown at left, with a magnification of the −6 charge state (shaded box) shown at right and with overlaid calculated isotope distributions for the indicated product chemical formula (red boxes).

FIGS. 19A-19D show that NP-DNA primer extension activity is template-directed and yields nuclease-resistant products. FIG. 19A shows primer extension reactions on the +71 nt template carried out as in FIG. 12B, except using single nucleotide drop-out substrate mixes where indicated. Reaction mixes equilibrated at 55° C. were initiated by addition of a substrate mix containing all four nNTP substrates (nN) or one prepared in the absence of the indicated aminonucleotide substrate. For each indicated reaction time (10-40 min), samples were quenched as in FIG. 12B. Partially hydrolyzed products are shown with hydrolytic fragments apparent only in the extended NP-DNA region of the chimeric product (HOAc lanes), prepared by incubating the full length extension product from the “nN” reaction 1:2.5 with 1% (v/v) HOAc in 98% formamide, 10 mM EDTA at 85° C. for 10 min (1). For all reactions, samples were quenched at the indicated times and then analyzed using a 15% TBE-urea gel (P: unextended primer). FIG. 19B shows single-nucleotide drop-out reactions as in FIG. 19B, except prepared using the +28 template. FIG. 19C shows preparation of a single-stranded chimeric DNA/NP-DNA product using a terminally-biotinylated template. Extension reactions were carried out using a biotinylated +11 template, otherwise as in FIG. 18, and the extended product duplex was isolated using a streptavidin-bound magnetic bead. After washing, the product strand was eluted using 0.1 N NaOH, followed by neutralization and desalting (see Materials and Methods). FIG. 19D shows single-stranded products prepared as in FIG. 19C, with either NP-DNA or DNA extensions were incubated with 1 U exonuclease I for the indicated times at 37° C. Samples were quenched and analyzed on a 15% TBE-urea gel (P-NH2: unextended 3′ amino-terminal primer. P-OH: unextended DNA primer).

The details of one or more embodiments of the invention are set forth in the description below. Other features or advantages of the present invention will be apparent from the following drawings and detailed description of several embodiments, and also from the appended claims.

### DETAILED DESCRIPTION

The present disclosure provides, in some aspects, improved methods for enzymatic synthesis of oligonucleotides or polynucleotides comprising phosphoramidate-linked nucleotides, and compositions comprising such. The enzymatic methods described herein advantageously resulted in massively greater numbers, over 100, of consecutive phosphoramidate-linked nucleotides in the oligonucleotide or the polynucleotide produced. The reason for this improvement in the enzymatic synthesis is due to the replacement of divalent Ca²⁺ as a cofactor with a trivalent cofactor, which are not typically used as cofactors by phosphoryl transfer enzymes. The diamagnetic group three trivalent rare earth element cations scandium (Sc³⁺), yttrium (Y³⁺), and lutetium (Lu³⁺), as well as the post-transition metal ion indium (In3), all significantly improved single-nucleotide 3′-amino primer extension, as well as NP-DNA polymerization with all four nNTPs on mixed sequence templates. Sc³⁺, in particular, accelerates pre-steady-state rate constants for nCTP addition by ˜100-fold at 55° C. to 6.8±0.3 min⁻¹ vs. 0.069 min⁻¹ for Ca²⁺. It was also found that the Sc³⁺ thio effect was 0.58±0.07 (mean±SD) at 45 C, indicating a significant preference for phosphorothioate substrates to yield thiophosphoramidate (NPS) linkages. Interestingly, the catalyzed reaction remained highly specific (66-fold) for the Sp over Rp substrates at 45° C.

Provided herein are methods involving a DNA polymerase variant (e.g., a DNA polymerase variant comprising a F710Y mutation) that extends 3′-amino terminal primers in a template-directed manner to produce oligonucleotides or polynucleotides comprising N3′→N5′ phosphoramidate (NP) bonds. Methods described herein can comprise a trivalent metal ion as a cofactor. Accordingly, methods described herein utilize a DNA polymerase variant and a trivalent metal ion cofactor to produce oligonucleotides or polynucleotides comprising phosphoramidate-linked nucleotides. The trivalent metal ion cofactor can be for example scandium (Sc³⁺), yttrium (Y³⁺), lutetium (Lu³⁺), indium (In³⁺), Europium (Eu³⁺), Gadolinium (Gd³⁺), Yttrium (Yb³⁺), or Erbium (Er³⁺).

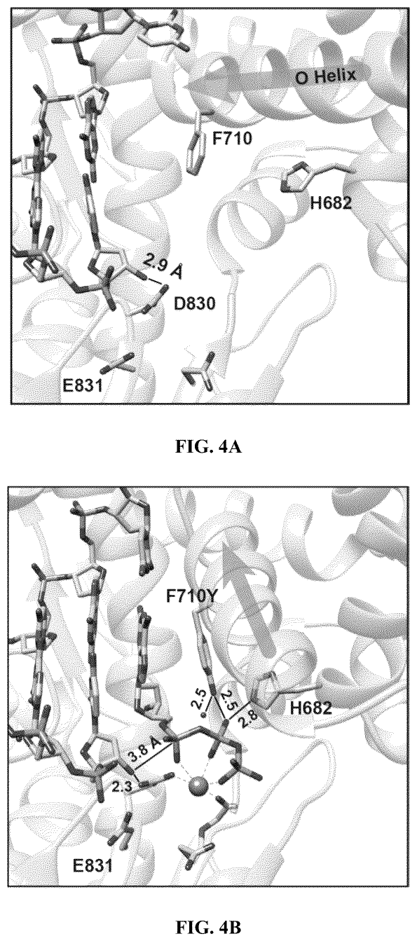

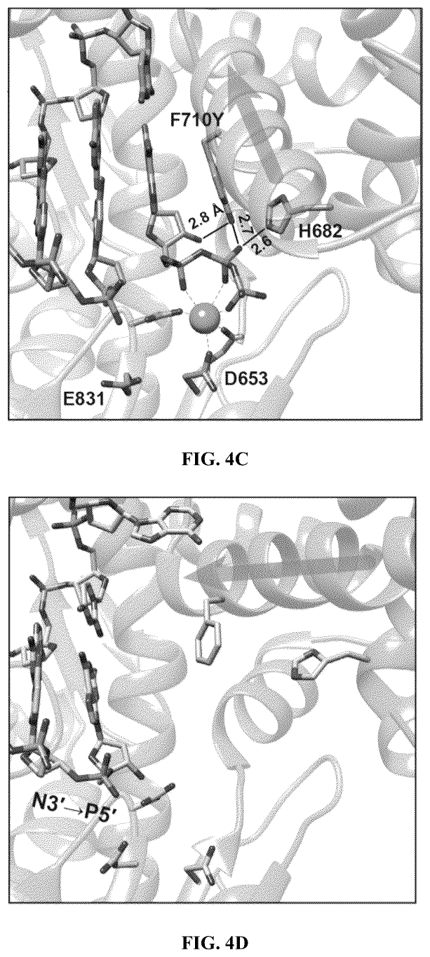

Studies described herein demonstrate that BF can use both deoxyribonucleoside triphosphate (dNTP) and 3′-amino-2′,3′-dideoxyribonucleoside triphosphate (nNTP) substrates in 3′-amino extension reactions. In some illustrative instances, BF was selective for dCTP over nCTP in 3′-amino extension reactions. This selectivity is modulated by mutations in the polymerase active site, notably the mutation F710Y in the “O-helix” of the fingers domain (Table 4). Without wishing to be bound by theory, this selectivity might be due, in part, from structural differences between the bound substrate geometry of nGTP seen in the crystal structure compared to that observed for the unreactive dGTP analog (FIGS. 4B-4C). Without wishing to be bound by theory, tuning of the reactivity of the substrate appears to be conferred not only by triphosphate-metal coordination, but also by side chains in the fingers domain that interact directly with the bound substrate in the closed conformation. However, use of a trivalent metal ion, for instance Sc³⁺, dramatically enhances enzyme specificity for synthesis of phosphoramidate and thiophosphoramidate linkages versus phosphodiester linkages.

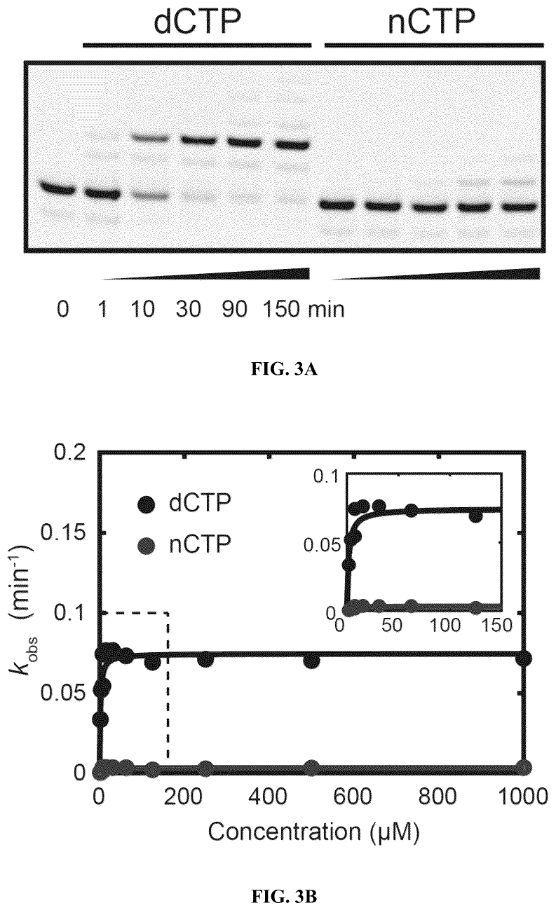

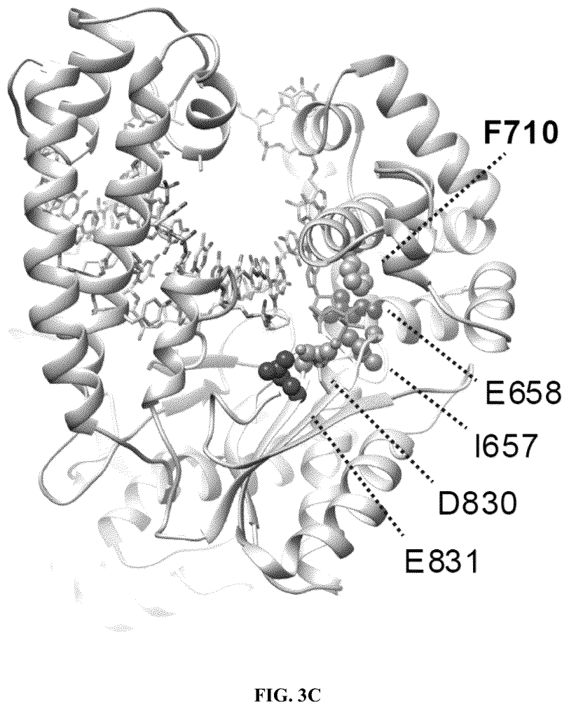

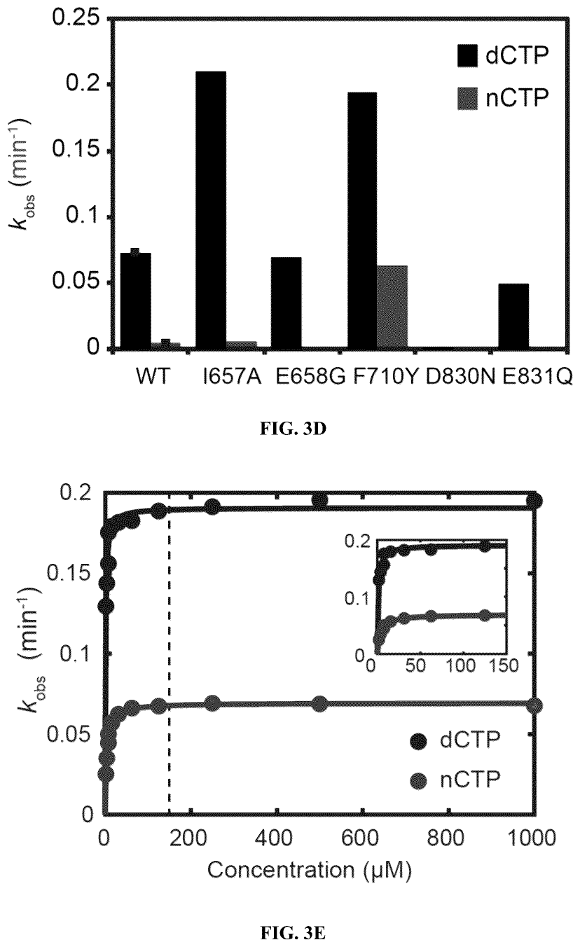

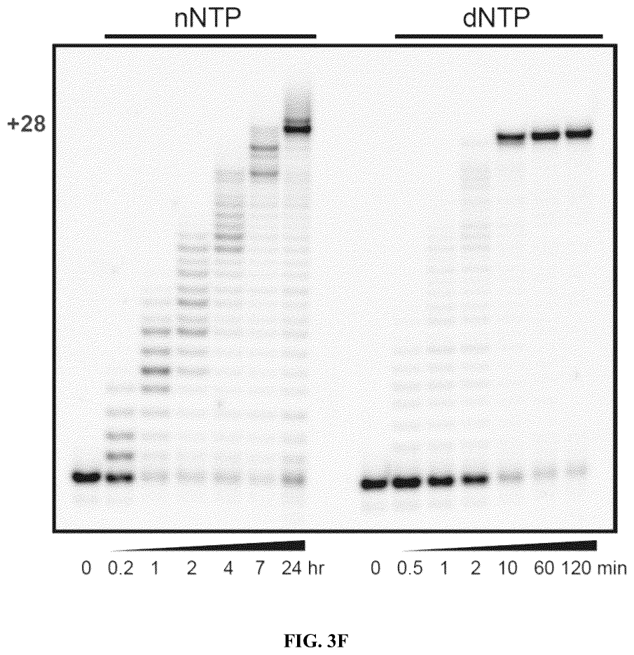

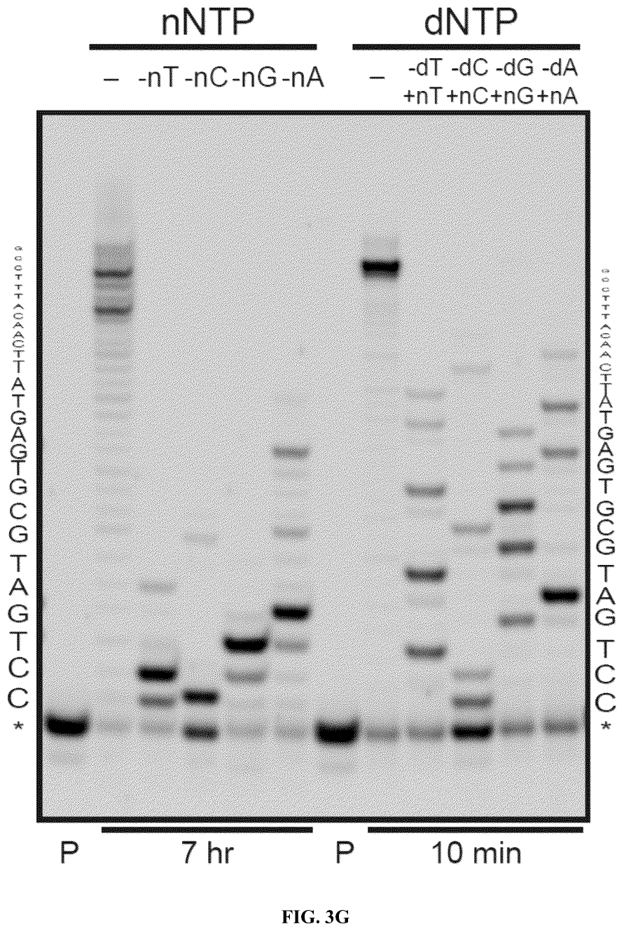

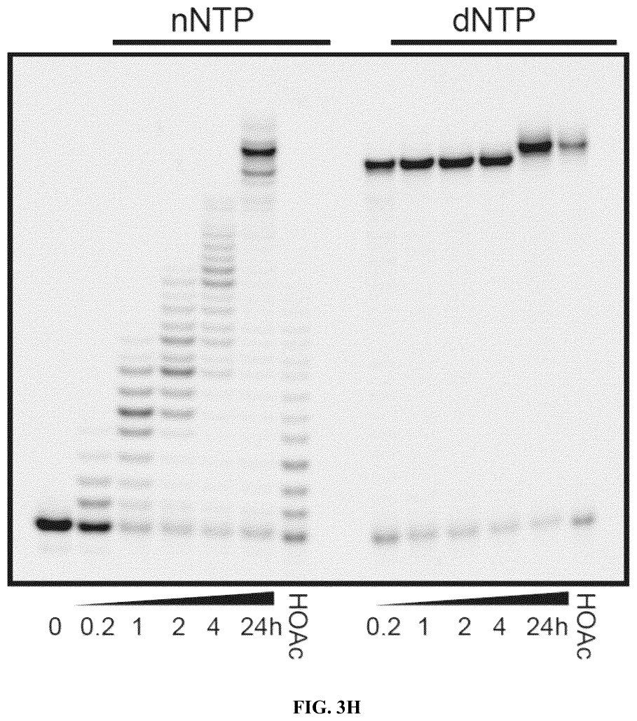

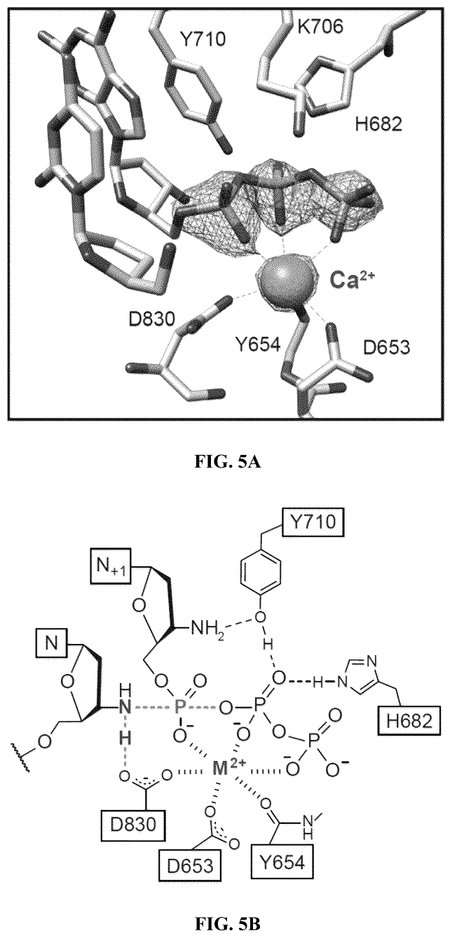

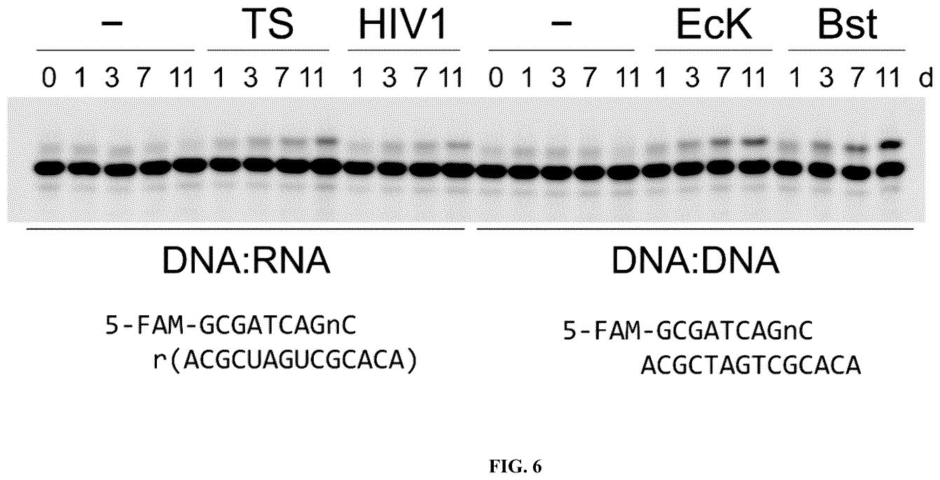

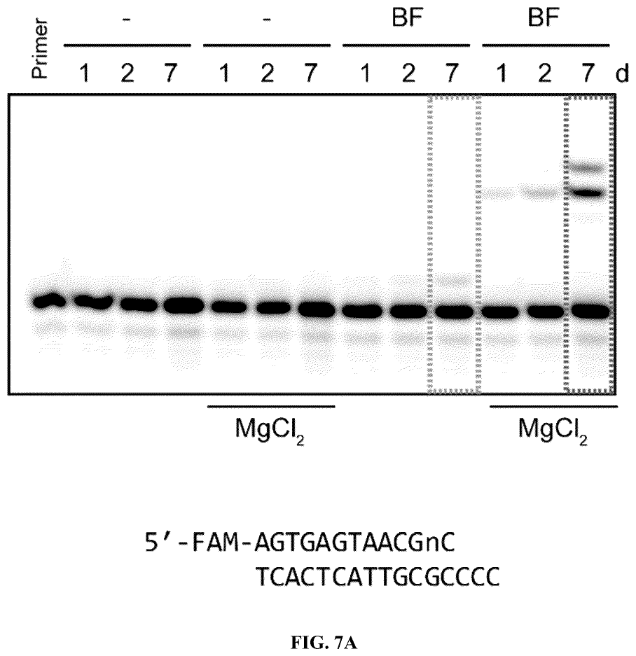

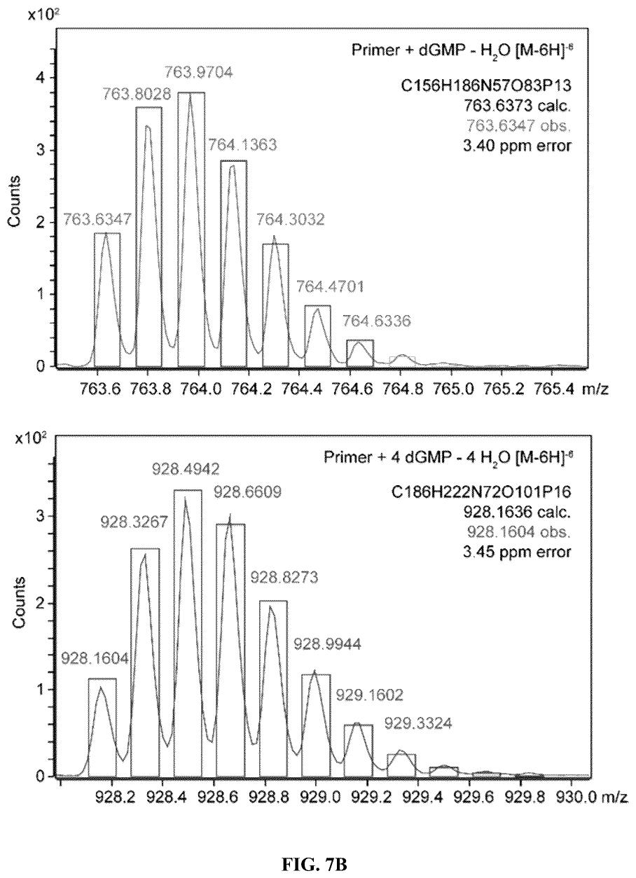

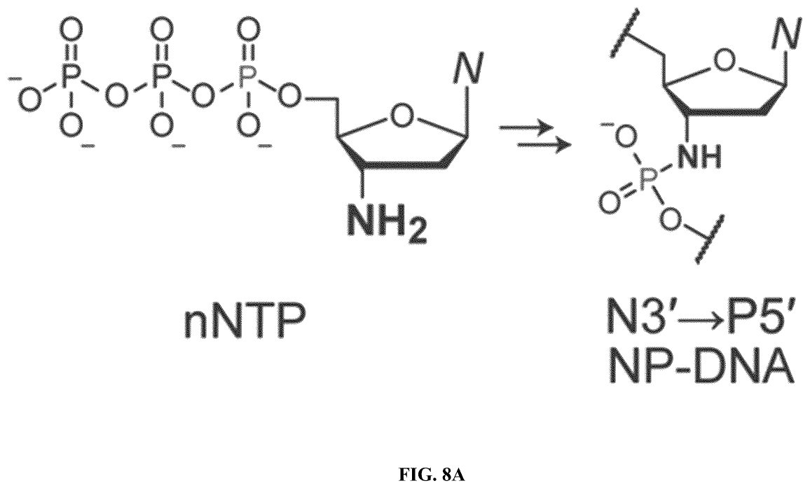

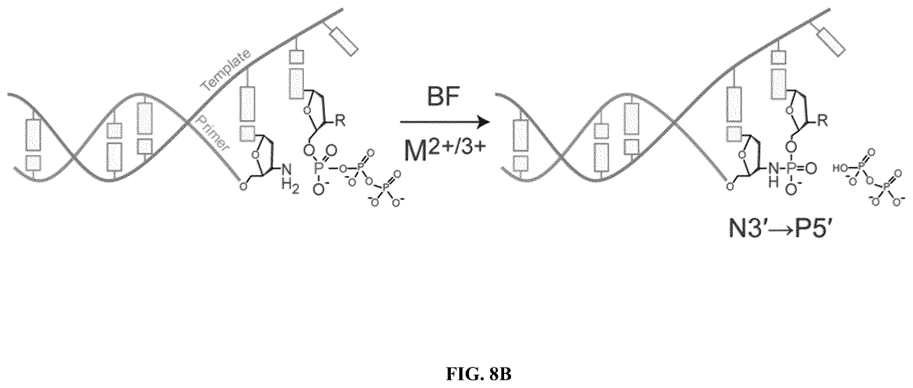

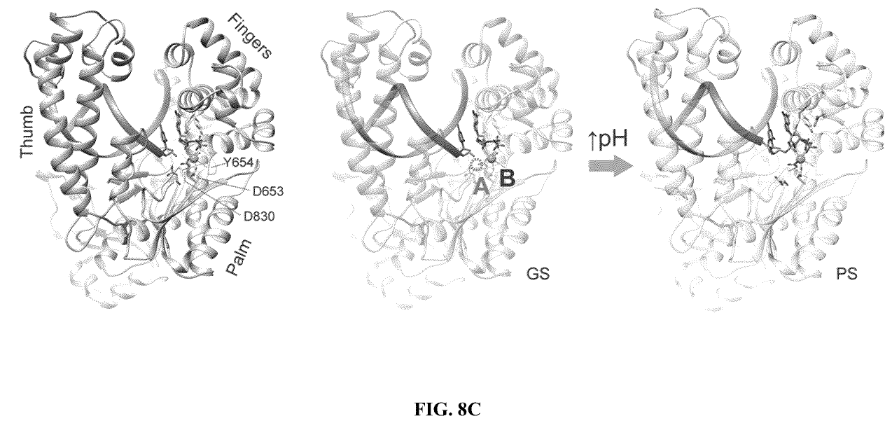

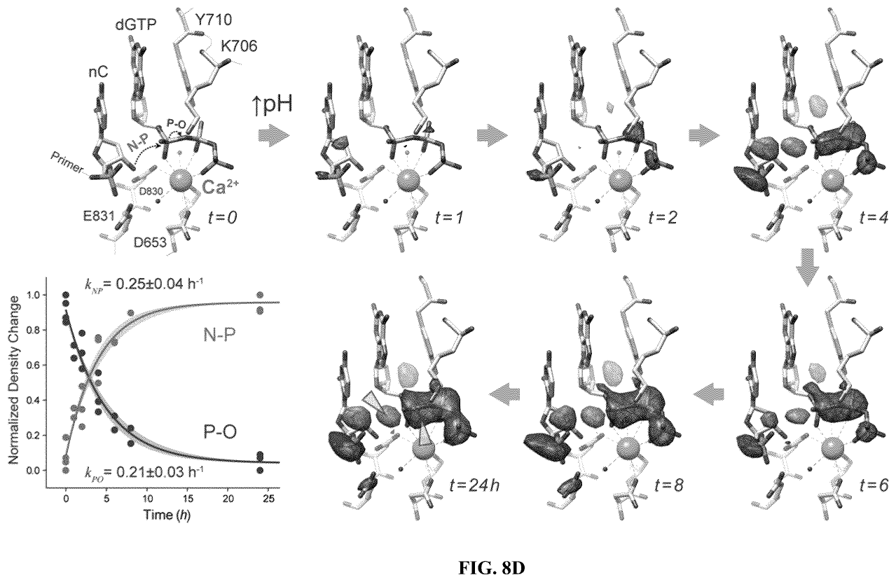

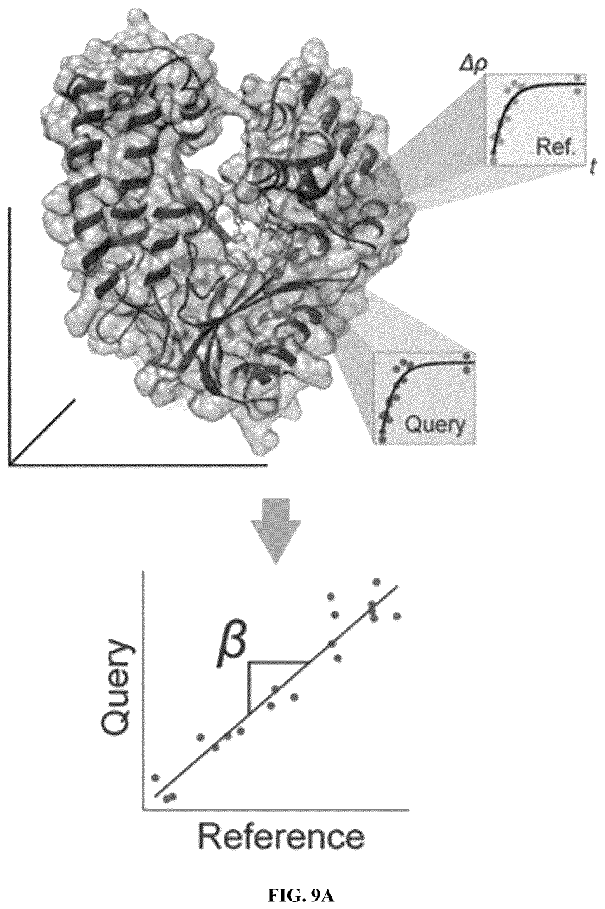

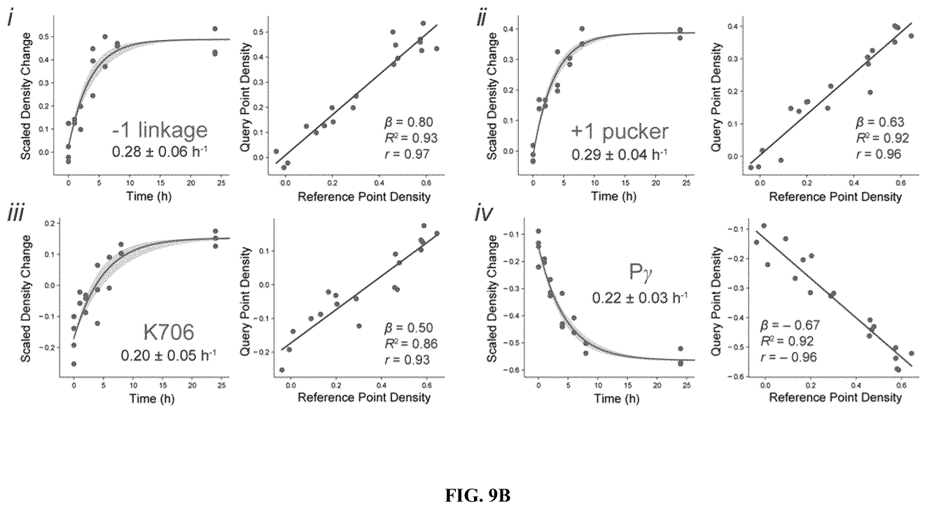

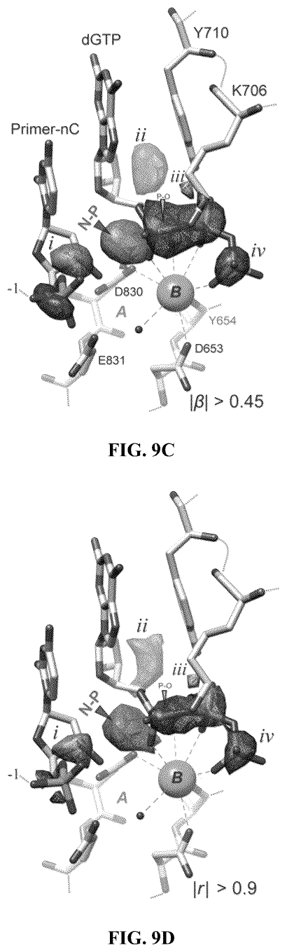

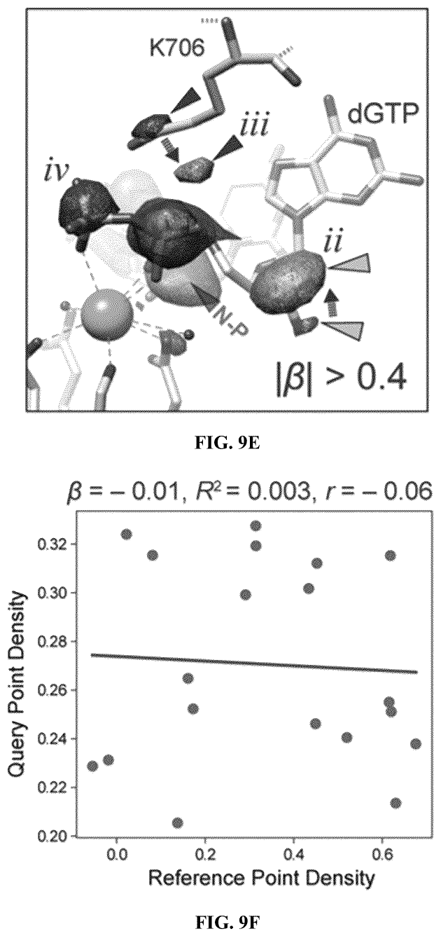

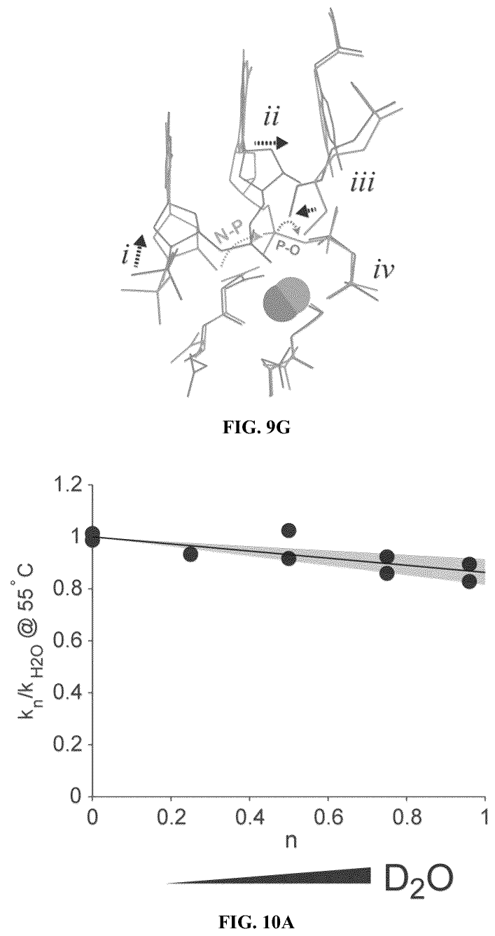

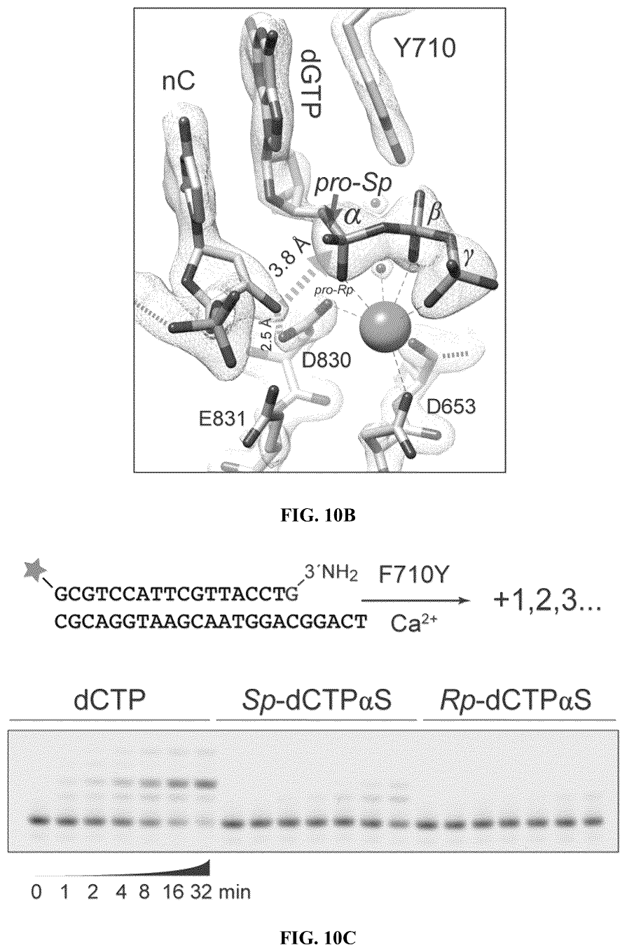

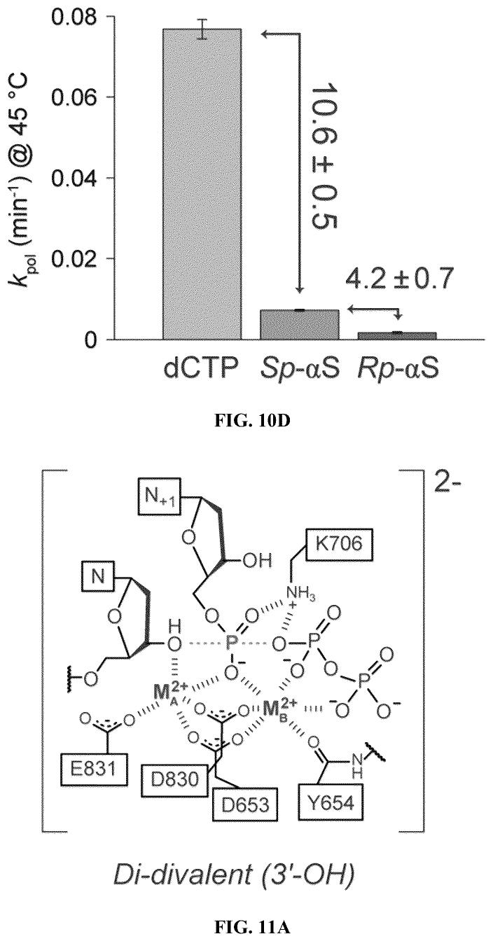

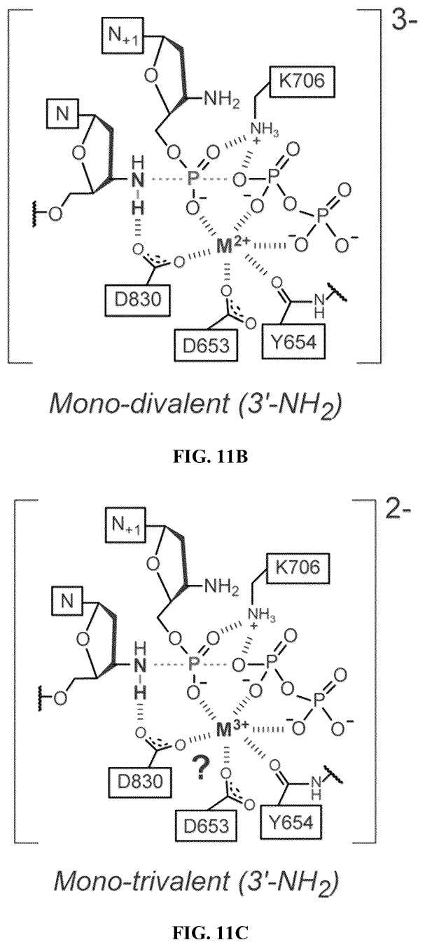

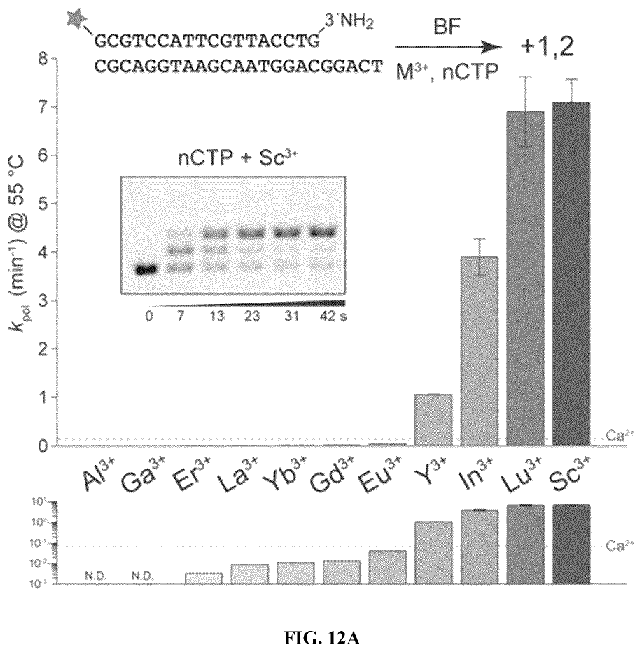

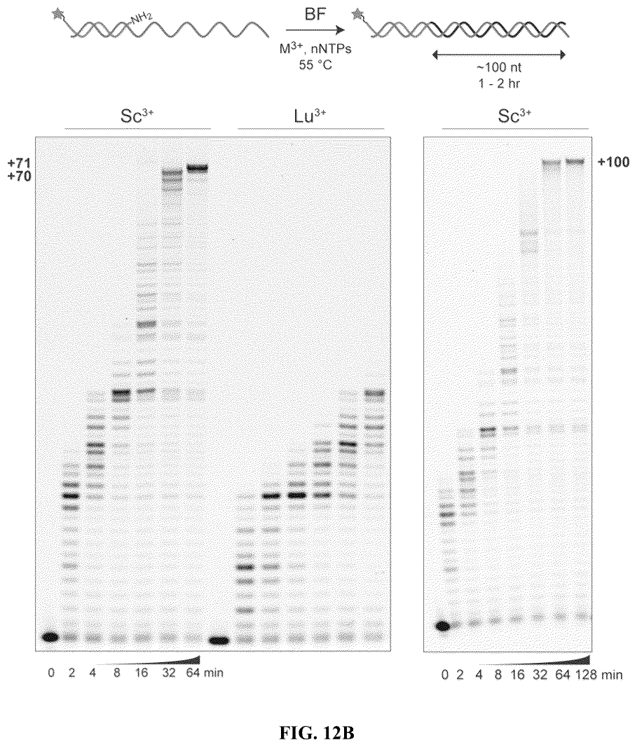

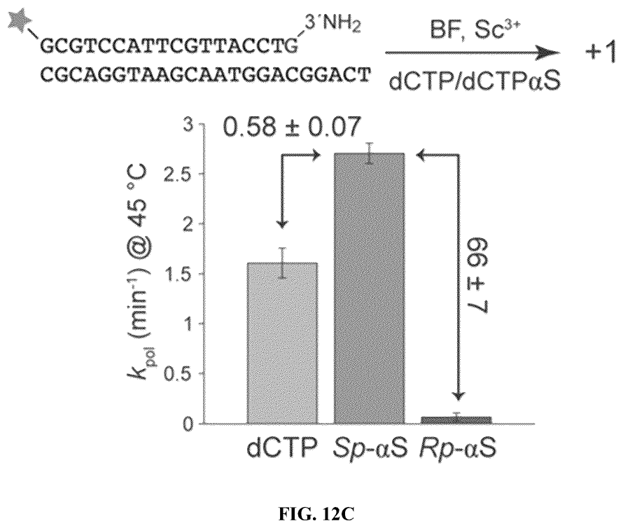

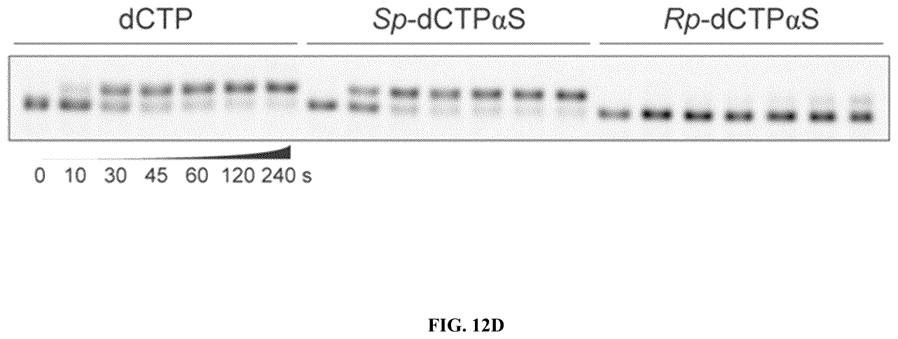

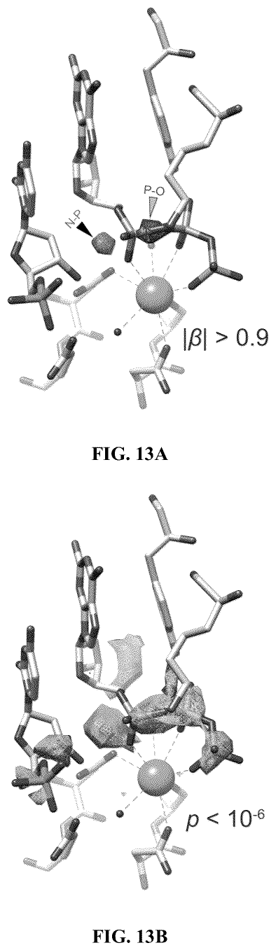

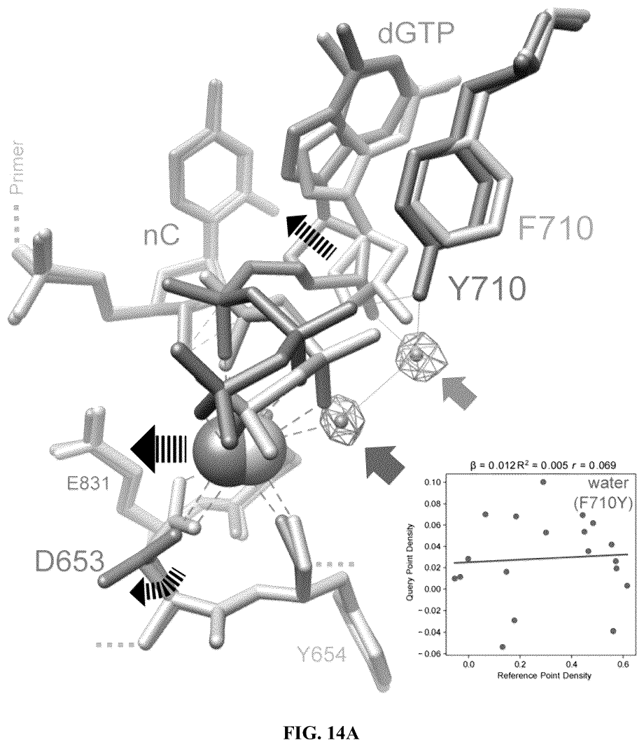

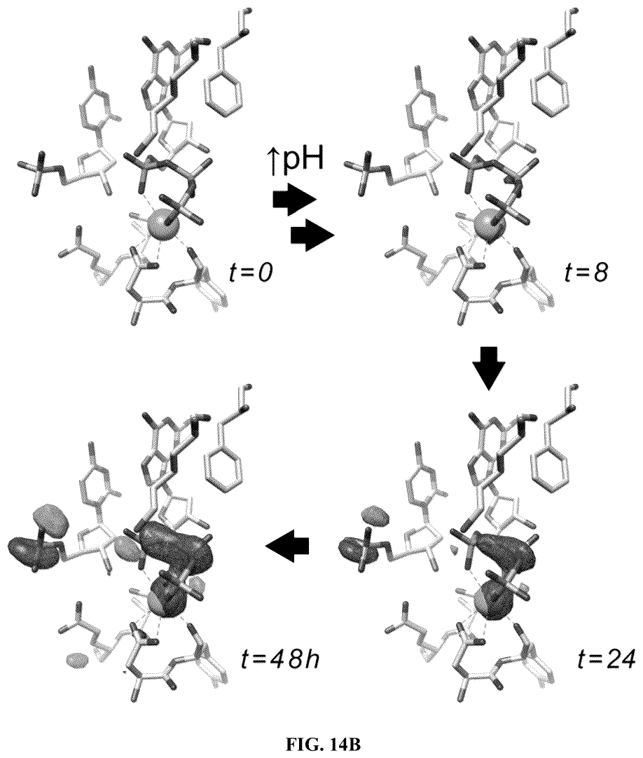

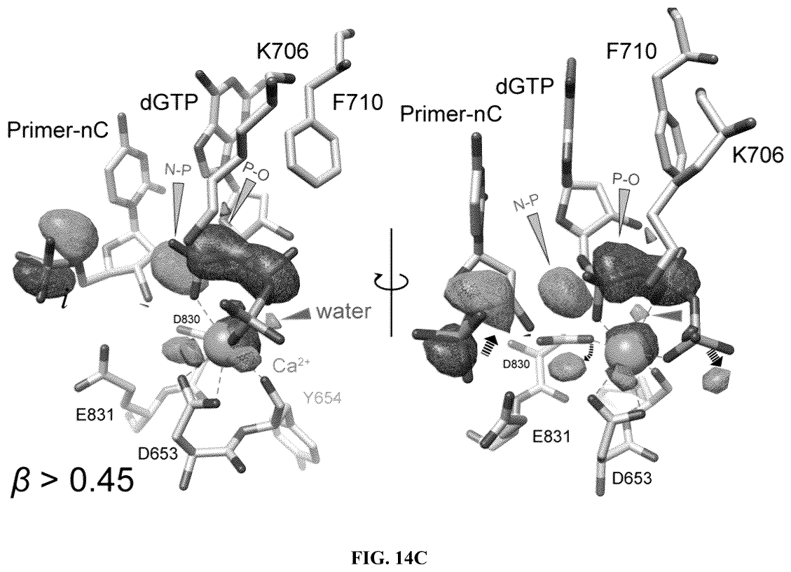

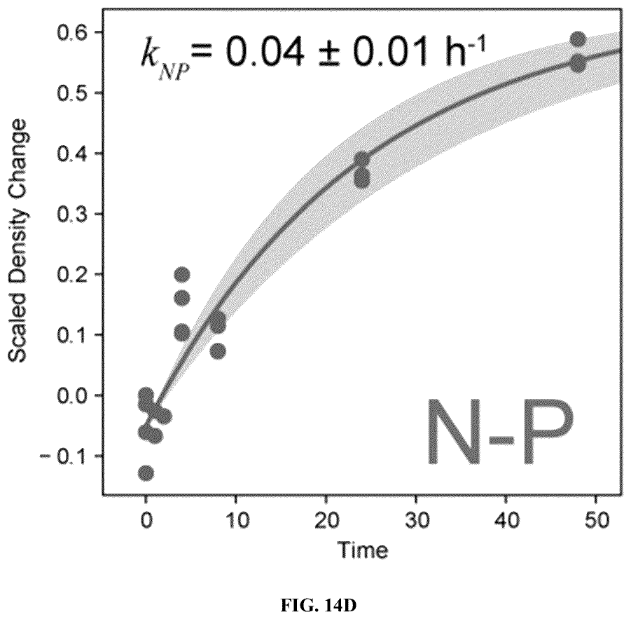

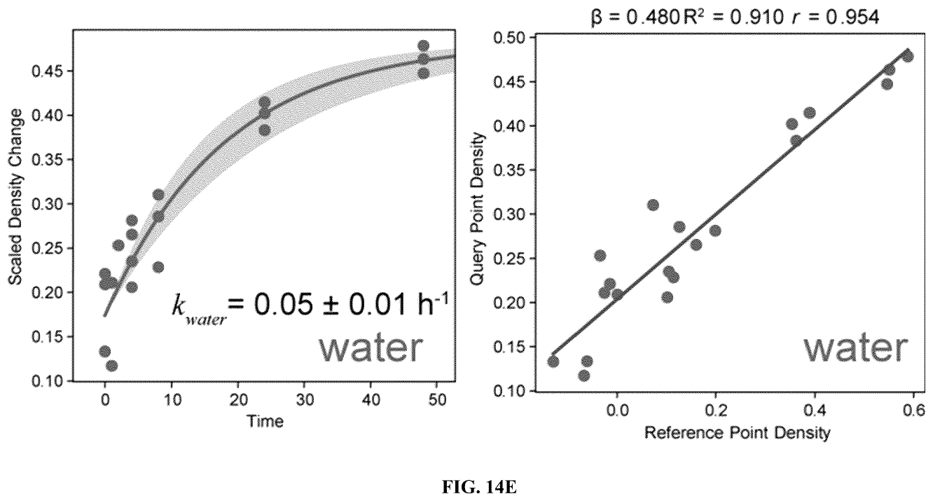

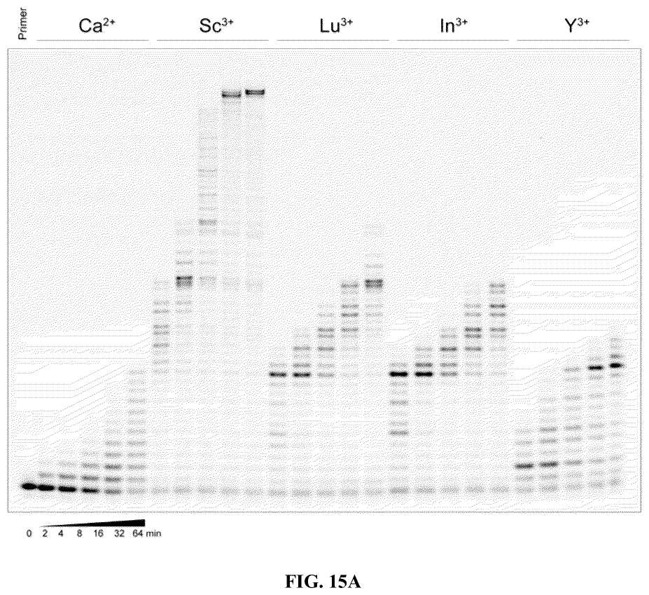

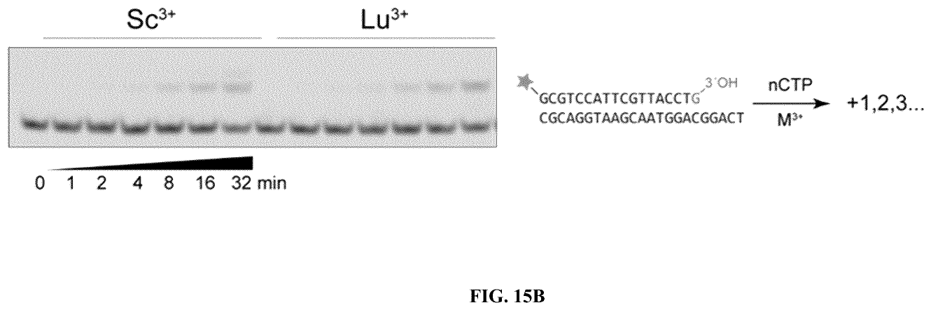

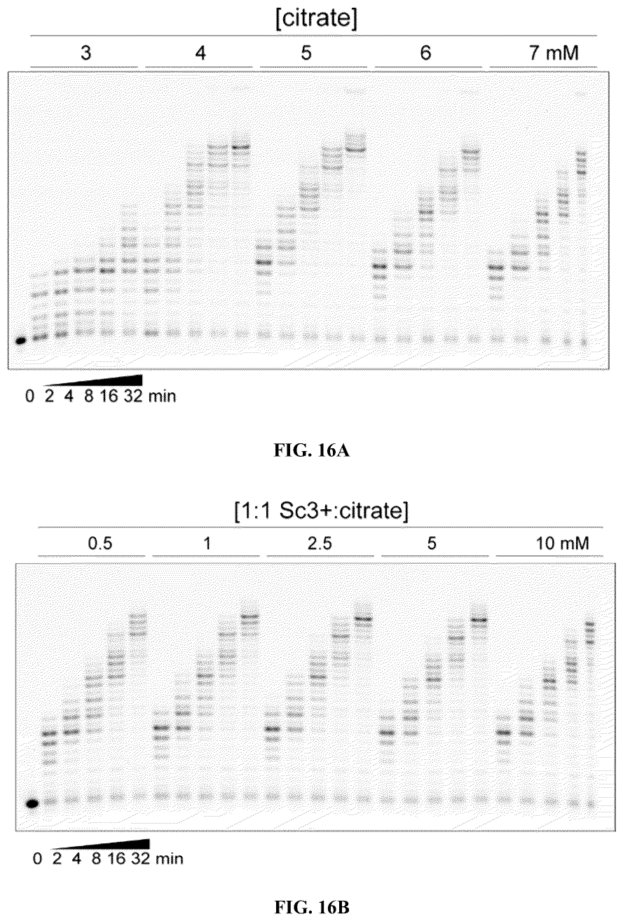

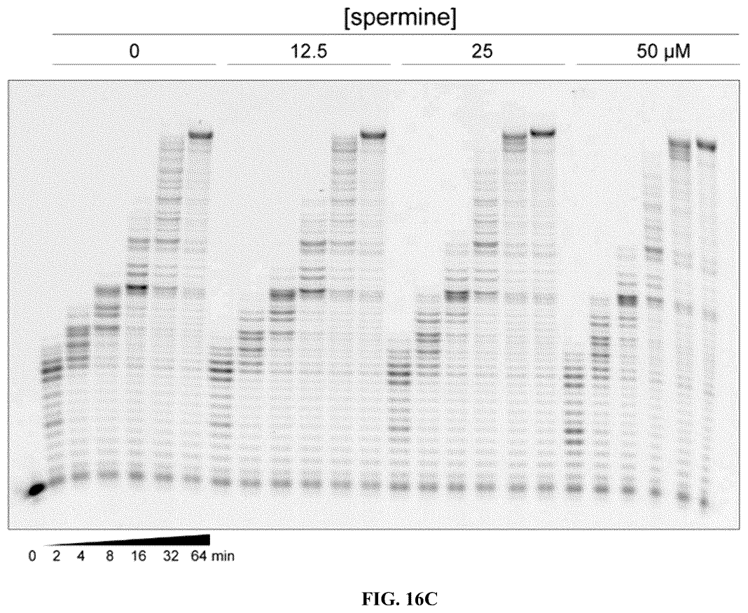

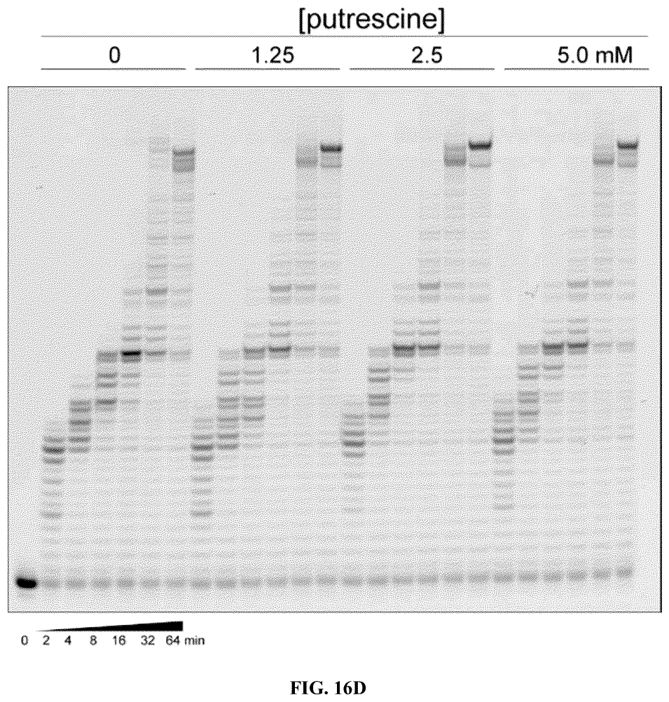

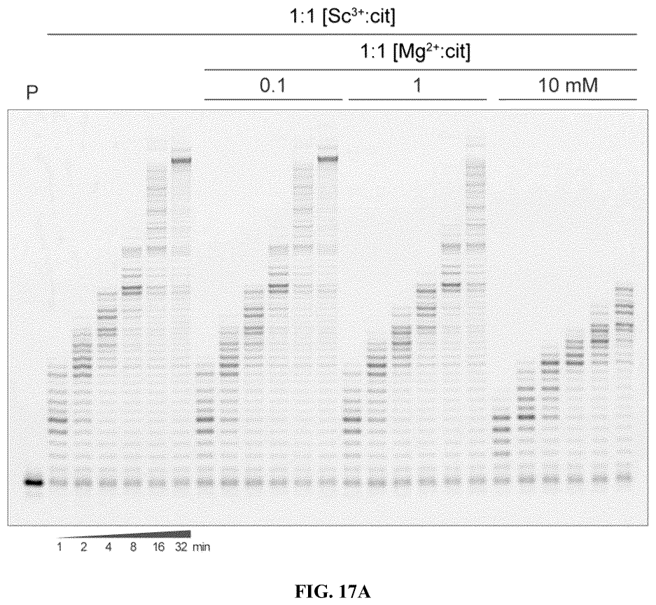

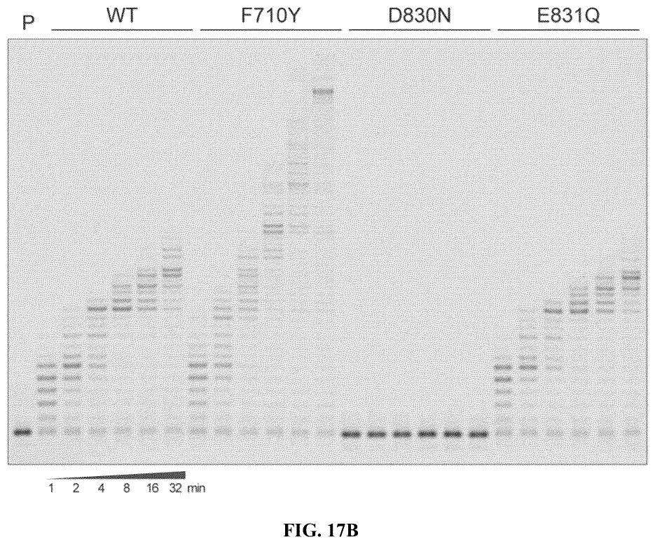

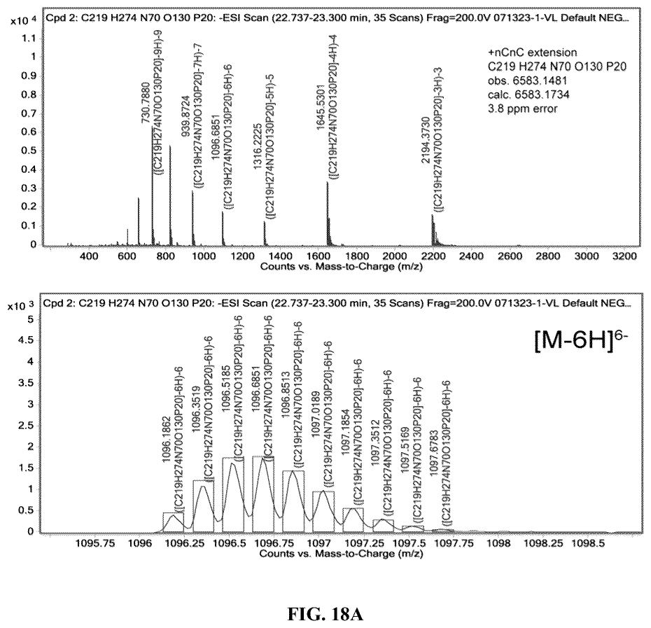

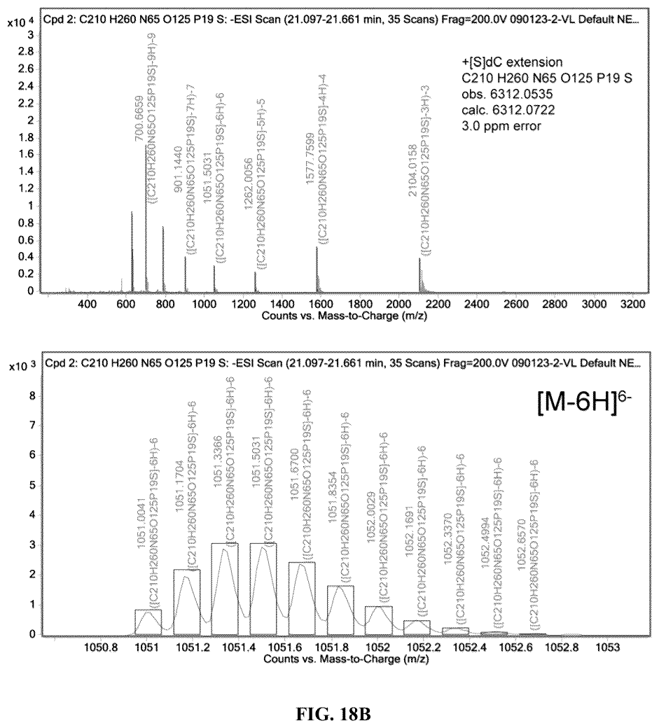

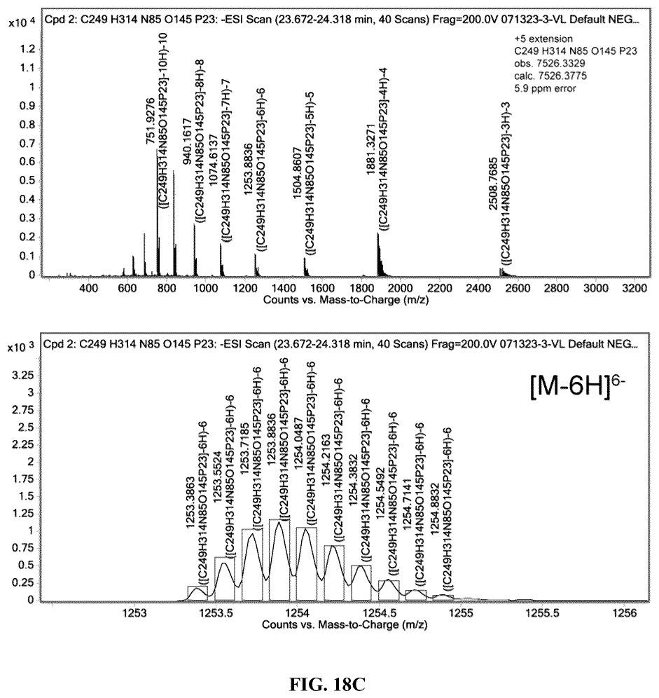

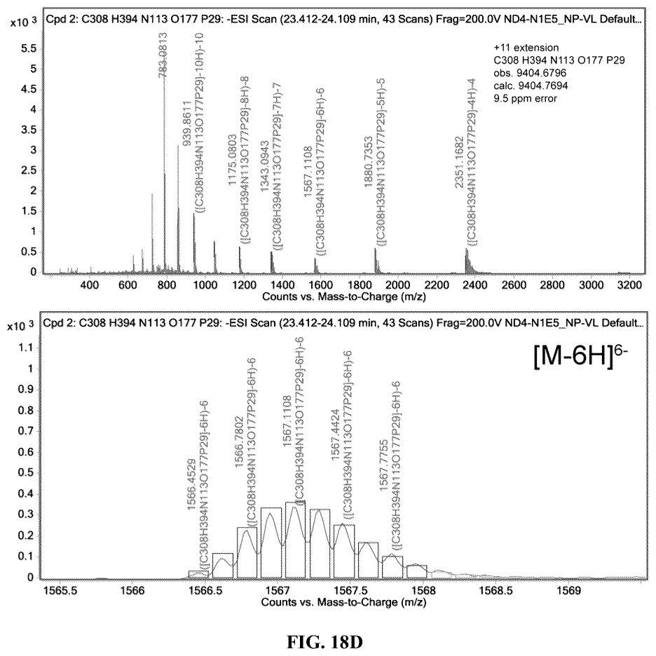

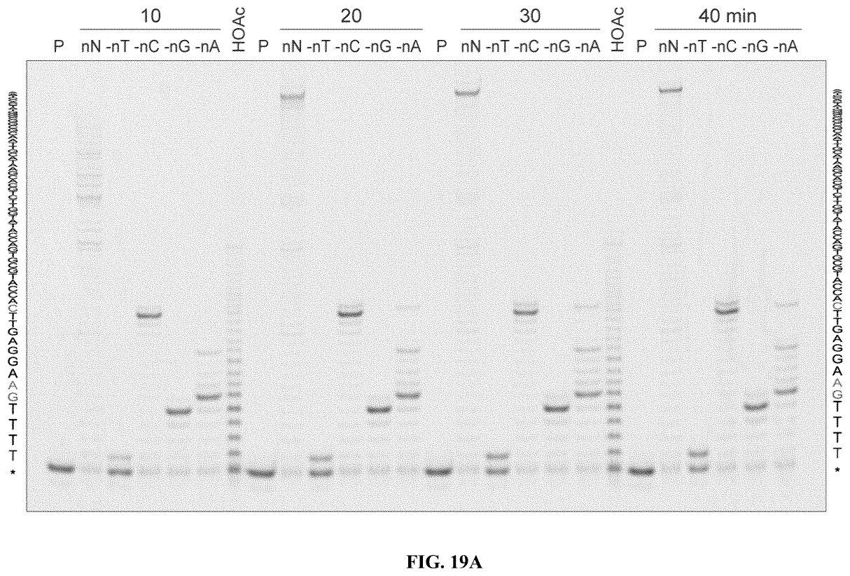

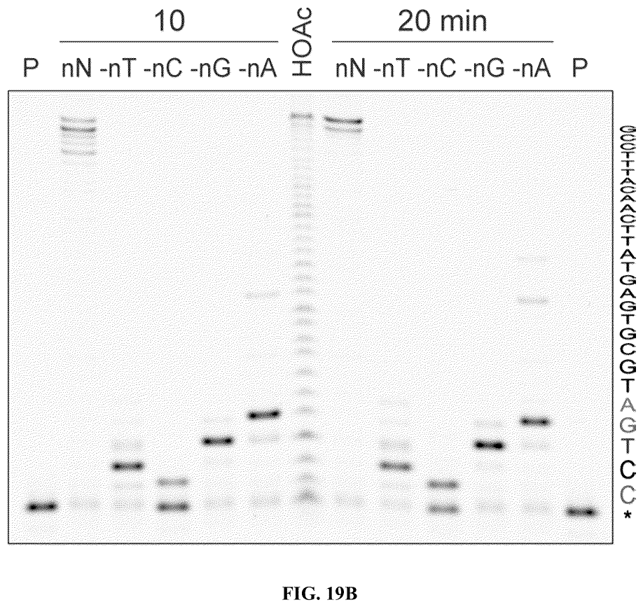

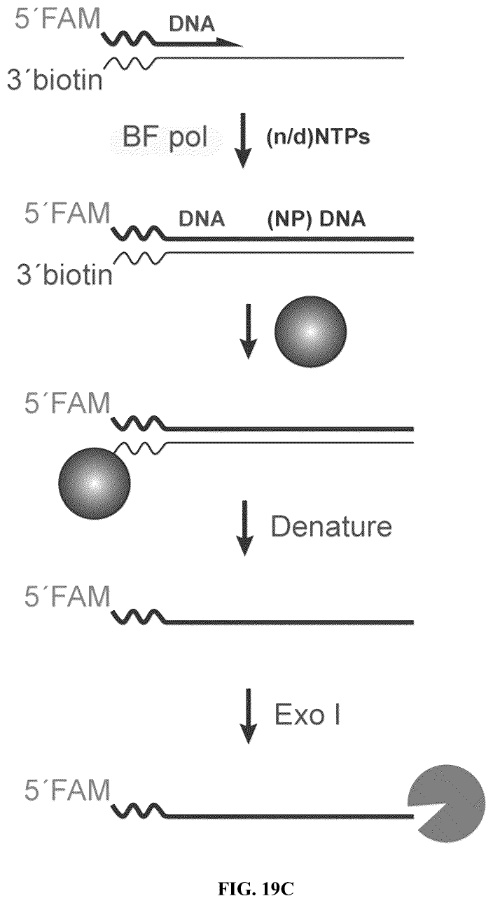

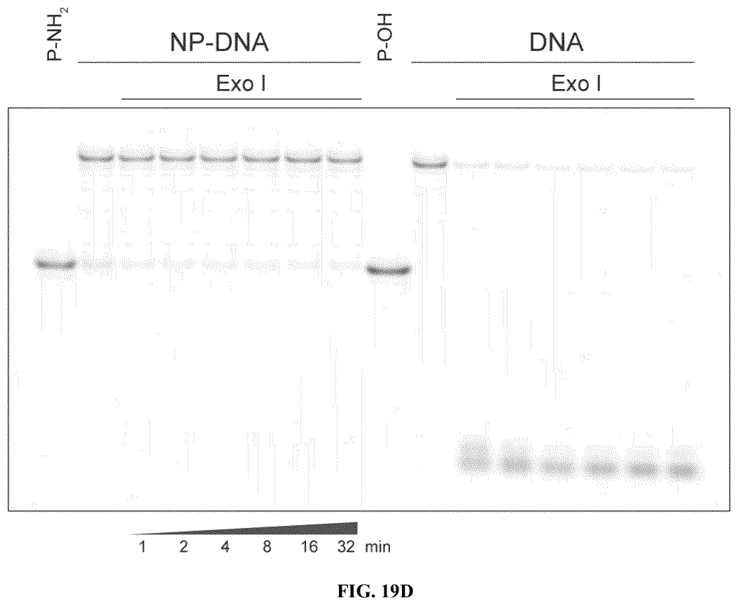

Phosphodiester bonds form the genetic backbone of life on Earth, but phosphoramidate esters do not. It should therefore be unsurprising that highly evolved polymerases are optimized for their wild-type activity and substrate, but it remains notable that 3′-amino nucleotides are not strictly chain terminators. Extension beyond a 3′-amino terminus proceeds with an alternative metal cofactor preference, suggesting mechanistic distinctions in the chemical step between N—P and O—P bond formation. Since the pKa of the protonated 3′-amino group is ˜7.5-7.7 as the free nucleoside (Cagri Izgu et al., Synthesis of activated 3′-amino-3′-deoxy-2-thio-thymidine, a superior substrate for the nonenzymatic copying of nucleic acid templates. Chem. Commun. 52, 3684-3686 (2016); and Kervio et al., Templating efficiency of naked DNA. Proc. Natl. Acad. Sci. 107, 12074-12079 (2010)), consistent with amino functionalities in other glycosylamines (S. Inouye, On the Prediction of pKa Values of Amino Sugars. Chem. Pharm. Bull. (Tokyo) 16, 1134-1137 (1968)), the amino nucleophile is expected to be substantially neutral under the reaction conditions. Notably, mutation of two acidic active site residues, which canonically bind a metal ion, activating the nucleophilic 3′-OH in the native O—P reaction, had disparate effects on the N—P reaction. The mutation D830N completely eliminated 3′-amino primer extension, but E831Q did not (FIG. 3D). The kinetics of these mutants, in addition to structural evidence, are more consistent with a role for Asp-830 as a general base than as a metal ligand proximal to the nucleophile (FIG. 5B).

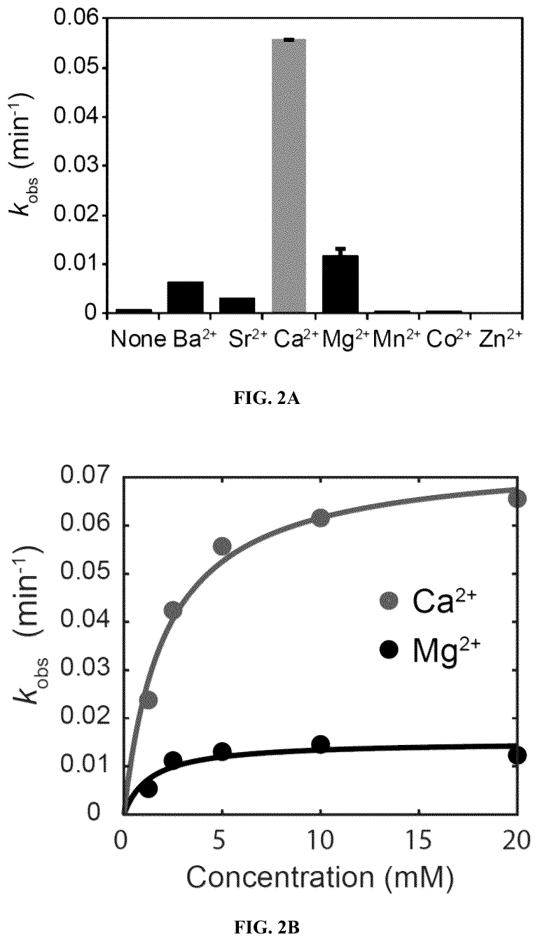

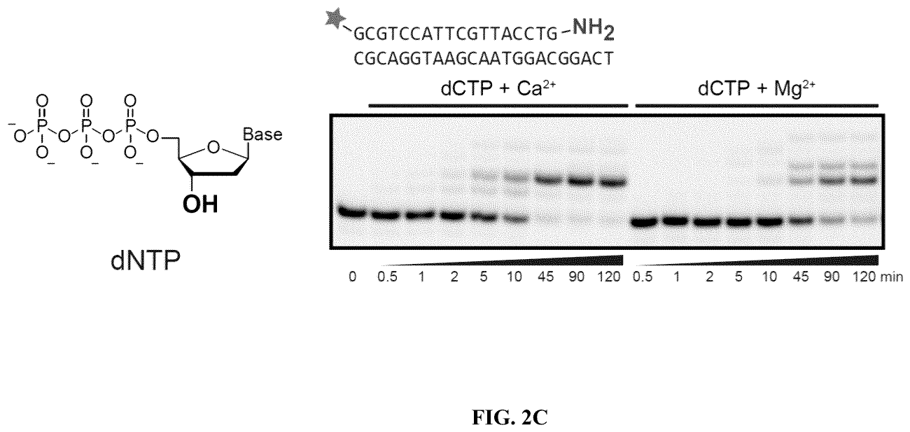

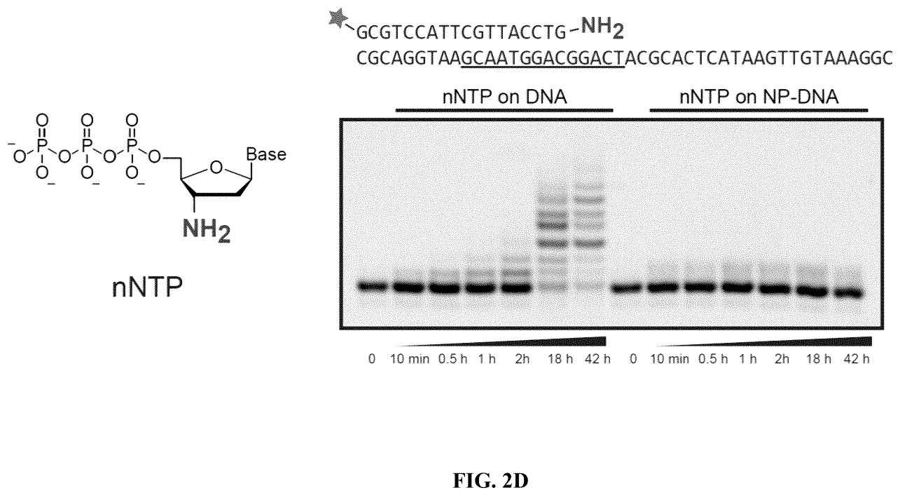

Results described herein demonstrate that this aspartate is likely to play a key role in facilitating proton transfer out of the transition state, in this case without the inner sphere nucleophile-metal ion coordination that is widely understood to activate the nucleophile in the corresponding phosphodiester forming reaction (Steitz et al., A unified polymerase mechanism for nonhomologous DNA and RNA polymerases—Comment/reply. Sci. Wash. 266, 2022 (1994)). It is therefore plausible that the distinction in proton transfer between the two reaction mechanisms contributes substantially to the observed kinetic defect. Some additional outer sphere role for divalent ions in the mechanism also cannot be ruled out, since pre-steady-state rates were not saturated at one equivalent of metal ion vs. substrate (FIGS. 2A-2D). Ca²⁺-mediated catalysis of DNA polymerase activity, which was observed in studies described herein, has been previously reported (Irimia et al., Calcium Is a Cofactor of Polymerization but Inhibits Pyrophosphorolysis by the Sulfolobus solfataricus DNA Polymerase Dpo4. Biochemistry 45, 5949-5956 (2006); and Ralec et al., Calcium-driven DNA synthesis by a high-fidelity DNA polymerase. Nucleic Acids Res. 45, 12425-12440 (2017)), but the precise role of Ca²⁺ in the phosphodiester-forming mechanism remains to be elucidated. By co-crystallization, Ca²⁺ bound to the substrate triphosphate moiety in a manner similar to that seen for Mg²⁺ crystallized under similar conditions was observed, but there remains the possibility that structures provided herein do not capture transiently bound metal ions that are nevertheless critical to the reaction mechanism, as has recently been proposed for certain eukaryotic polymerase family members (Gao et al., Capture of a third Mg²⁺ is essential for catalyzing DNA synthesis. Science 352, 1334-1337 (2016); and Freudenthal et al., Uncovering the polymerase-induced cytotoxicity of an oxidized nucleotide. Nature 517, 635-639 (2015)).

For NP-DNA to participate in any abiogenesis, e.g., a synthetic one undertaken in the laboratory, it would require that this alternative genetic polymer play informational and functional roles in the resulting cell. Given that DNA, RNA, and various XNAs can form folded structures with a broad range of catalytic activities stretching across sequence space, it is reasonable to expect that the sequence space of NP-DNA, too, will contain diverse functions. Although several key aspects of its viability as an alternative genetic polymer remain to be explored, it has long been known that NP-DNA forms stable duplexes with RNA and DNA (Gryaznov et al., Oligodeoxyribonucleotide N3′→P5′ Phosphoramidates: synthesis and Hybridization Properties. J. Am. Chem. Soc. 116, 3143-3144 (1994)), implying its capacity to exchange information. Among alternative nucleic acids known to pair with RNA, only a subset (Kim et al., A model for the emergence of RNA from a prebiotically plausible mixture of ribonucleotides, arabinonucleotides and 2′-deoxynucleotides. J. Am. Chem. Soc. 142, 5, 2317-2326 (2020)) are compatible with template-directed nonenzymatic copying chemistry based on phosphorimidazolides. Interestingly, NP-DNA now appears to be compatible with both nonenzymatic phosphorimidazolide and enzymatic triphosphate chemistries. As a result, a genetic transition between backbone linkages is biochemically conceivable with either chemistry.

### I. Reaction Components for Producing Oligonucleotides or Polynucleotides Comprising Phosphoramidate-Linked or Thiophosphoramidate-Linked Nucleotides

Methods described herein involve the use of a DNA polymerase variant in a pH-buffered aqueous reaction mixture consisting of a divalent metal ion cofactor and/or a trivalent metal ion cofactor, nucleotides (e.g., 3′-amino-2′,3′-dideoxyribonucleoside 5′-triphosphates (nNTPs)), a 3′-amino terminated primer, and a DNA template, to enzymatically produce oligonucleotides or polynucleotides comprising phosphoramidate-linked or thiophosphoramidate-linked nucleotides.

### DNA Polymerase Variants

A DNA polymerase variant, as used herein, refers to a DNA polymerase enzyme comprising a substitution at position F710 (e.g., F710Y) or an equivalent thereof. The DNA polymerase variant can comprise the amino acid sequence of a DNA polymerase from any source, e.g., the amino acid sequence of the DNA polymerase from the thermophilic bacterium Bacillus stearothermophilus. It should be understood that the amino acid substitution at position F710 in the DNA polymerase from B. stearothermophilus can be incorporated into DNA polymerases from various sources with similar effects on the phosphoramidate bond formation activity of the DNA polymerase. In such instances, the substitution can be at a position other than position 710. For example, as shown in Table 1 below, the substitution equivalent to the substitution at position F710 in the DNA polymerase from B. stearothermophilus is at position 762 in the DNA polymerase from Escherichia coli.

Non-limiting examples of DNA polymerase variants that can be used to produce oligonucleotides or polynucleotides comprising phosphoramidate-linked nucleotides, as provided herein, are provided in Table 1.

TABLE 1
Examples of DNA Polymerases Comprising a Substitution
at Position F710 or an equivalent thereof.

| | | | Catalytic | | |
|---|---|---|---|---|---|
| | | Equivalent | domain start | | |
| | | codon to | (C-terminal | Accession | Ref |
| Organism | Gene | Bst 710 | fragment start) | (Uniprot) | (mutation) |
| Geobacillus | POLA | 710 | 469 | P52026, | Described herein |
| stearothermophilus | | | | D9N168 | |
| Thermus aquaticus | POLA | 667 | 423 | P19821 | U.S. Pat. No. 5,614,365A |
| Escherichia coli | POLA | 762 | 521 | P00582 | U.S. Pat. No. 5,614,365A |
| Thermus thermophilis | POLA | 669 | 427 | P52028 | Asakura et al. |
| Homo sapiens | POLG | 951 | 738 | P54098 | Lestienne P. |
| Bacteriophage T5 | dpol | 596 | 333 | P19822 | Chatterjee et al. |
| Bacteriophage T7 | gp5 | 526 | 211 | P00581 | U.S. Pat. No. 5,614,365A |

References:
Asakura et al., Cloning, nucleotide sequence, and expression in Escherichia coli of DNA polymerase gene (polA) from Thermus thermophilus HB8. J. Ferment. Bioeng. 76, 265-269 (1993).
Lestienne P., Evidence for a direct role of the DNA polymerase gamma in the replication of the human mitochondrial DNA in vitro. Biochem. Biophys. Res. Commun. 146 (3), 1146-1153 (1987).
Chatterjee et al., Cloning and overexpression of the gene encoding bacteriophage T5 DNA polymerase. Gene 97 (1), 13-19 (1991).

The DNA polymerase variant can comprise a full-length protein or a fragment thereof. In some examples, the DNA polymerase variant comprises the full-length amino acid sequence of the DNA polymerase from B. stearothermophilus. In other examples, the DNA polymerase variant comprises a fragment of the full-length amino acid sequence of the DNA polymerase from B. stearothermophilus, e.g., a fragment comprising amino acids 298-876, which is provided below as SEQ ID NO: 1.

(SEQ ID NO: 1)
KMAFTLADRVTEEMLADKAALVVEVVEENYHDAPIVGIAVVNEHGRFFL
RPETALADPQFVAWLGDETKKKSMFDSKRAAVALKWKGIELCGVSFDLL
LAAYLLDPAQGVDDVAAAAKMKQYEAVRPDEAVYGKGAKRAVPDEPVLA
EHLVRKAAAIWELERPFLDELRRNEQDRLLVELEQPLSSILAEMEFAGV
KVDTKRLEQMGKELAEQLGTVEQRIYELAGQEFNINSPKQLGVILFEKL
QLPVLKKTKTGYSTSADVLEKLAPYHEIVENILHYRQLGKLQSTYIEGL
LKVVRPDTKKVHTIFNQALTQTGRLSSTEPNLQNIPIRLEEGRKIRQAF
VPSESDWLIFAADYSQIELRVLAHIAEDDNLMEAFRRDLDIHTKTAMDI
FQVSEDEVTPNMRRQAKAVNFGIVYGISDYGLAQNLNISRKEAAEFIER
YFESFPGVKRYMENIVQEAKQKGYVTTLLHRRRYLPDITSRNFNVRSFA
ERMAMNTPIQGSAADIIKKAMIDLNARLKEERLQAHLLLQVHDELILEA
PKEEMERLCRLVPEVMEQAVTLRVPLKVDYHYGSTWYDAK

The DNA polymerase variant can comprise a mutation in addition to an amino acid substitution at position F710 or an equivalent thereof. Such a mutation can be an insertion, a deletion, a substitution, or a combination thereof. For example, the DNA polymerase variant can comprise an amino acid sequence that is at least 70%, at least 80%, at least 85%, at least 90%, at least 91%, at least 92%, at least 93%, at least 94%, at least 95%, at least 96%, at least 97%, at least 98%, or at least 99% identical to SEQ ID NO: 1, which comprises a substitution at position F710 (e.g., F710Y). In some embodiments, the DNA polymerase variant comprises the amino acid sequence set forth in SEQ ID NO: 1, which comprises a substitution at position F710 (e.g., F710Y).

The DNA polymerase variant to be used in methods described herein can be prepared by any known method. The DNA polymerase variant can be added to a reaction mixture as a recombinant protein and/or as a cell lysate from cells that express the DNA polymerase variant. In some embodiments, the DNA polymerase variant can comprise a fusion protein or a tagged protein (e.g., a His6-tagged DNA polymerase variant).

### Divalent Metal Ion Cofactors

A divalent metal ion cofactor can be used by the DNA polymerase variant to form a phosphodiester linkage between a nucleotide (e.g., nNTP) and the terminal nucleotide of the 3′-amino terminated primer, which can be referred to as a single turnover extension of the 3′-amino terminated primer. The DNA polymerase variant can then use a trivalent metal ion cofactor for multiple turnover extension of the 3′-amino terminated primer. Non-limiting examples of divalent metal ion cofactors include Ba2+, Sr+, Ca²⁺, Mg²⁺, Mn²⁺, Co²⁺, and Zn²⁺. In some embodiments, the divalent metal ion cofactor is Ca²⁺.

### Trivalent Metal Ion Cofactors

A trivalent metal ion cofactor enhances the catalytic efficiency and specificity of the DNA polymerase variant in synthesis of phosphoramidate-linked oligonucleotides or polynucleotides as compared to the previously used divalent metal ion cofactors disclosed in PCT Patent Publication WO2021/108632. Non-limiting examples of trivalent metal ion cofactors include trivalent lanthanide metals (e.g., Er³⁺, Gd³⁺, Eu³⁺, Yb³⁺), trivalent transition metals (e.g., Al³⁺, Cr³⁺, Au³⁺), trivalent group III metals (e.g., Y³⁺, Sc³⁺), and trivalent post-transition series metals (e.g., In³⁺). Non-limiting examples of trivalent metal ion cofactors include Sc³⁺, Y³⁺, Lu³⁺, In³⁺, Eu³, Gd³⁺, Yb³⁺ and Er³⁺. In some embodiments, the trivalent metal ion cofactor is Sc³⁺.

### Metal Chelating Agents

Methods described herein can involve use of a metal chelating agent. Non-limiting examples of metal chelating agents include citrate (e.g., ammonium citrate, magnesium citrate, sodium citrate), isocitrate, nitrilotriacetic acid (NTA), nitrilotriacetate, malate, malonate, maleate, oxalate, adipate, glutarate, succinate, fumarate, tripolyphosphate (TPP) sodium salt, or combinations of any of these.

### Polyamine Additives

Addition of a polyamine to the enzymatic reaction appreciably enhances the synthesis of phosphoramidate-linked oligonucleotides or polynucleotides. As used herein, the term “polyamine” refers to molecules comprising two or more amines. Polyamines can be aliphatic, straight-chain amines derived biosynthetically from amino acids. Several polyamines are reviewed in Marton et al. (1995) Ann. Rev. Pharm. Toxicol. 35:55-91, content of which is incorporated herein by reference. Non-limiting examples of polyamines include spermine, putrescine, spermidine, cadaverine, thermospermine, caldopentamine, and caldohexamine. In some embodiments, the polyamine is spermine.

Methods described herein can include use of a polyamine analog. “Polyamine analog” is defined as an organic cation structurally similar but non-identical to polyamines such as spermine and/or spermidine and their precursor, diamine putrescine. Polyamine analogs, which can be branched or unbranched, include, but are not limited to, BE-4444 [1,19bis(ethylamino)-5,10,15-triazanonadecane]; BE-333 [N1, N11-diethylnorspermine; DENSPM; 1,11-bis(efhylamino)4,8-diazaundecane; thermine; Warner-Parke-Davis]; BE-33 [N1,N7-bis(ethyl) norspermidine]; BE-34 [N1,N8-bis (ethyl) spermidine]; BE-44 [N1,N9-bis (ethyl) homospermidine]; BE-343 [N1,N12-bis (ethyl) spermine; diethylspermine-N1-N12; DESPM]; BE-373 [N,N′-bis(3-ethylamino) propyl)-1,7-heptane diamine, Merrell-Dow]; BE-444 [N1, N14-bis (ethyl) homospermine; diefhylhomospermine-N1N14]; BE-3443 [1,17-bis (ethylamino)-4,9,14-triazaheptadecane]; BE-4334 [1,17-bis (ethylamino)-5,9,13triazaheptadecane]; 1,1 2-Me2-SPM [1,12dimethylspermine]; and the various polyamine analogs disclosed in WO 98/17624; U.S. Pat. No. 5,889,061; WO 00/66175 and WO 00/66587; and O'Sullivan et al. (1997) Bioorg. Med. Chem. 5:2145-2155 and Mukhopadhyaya et al. (1995) Exp. Parasit. 81:39-46; and U.S. Pat. No. 4,935,449, content of all of which is incorporated herein by reference.

Methods described herein can include use of a cyclic polyamine compound. Cyclic polyamine compounds and cyclic polyamine analogs are disclosed in International Patent Application WO 02/10142. In certain of these cyclic polyamine compounds, one or more of the aliphatic nitrogens form part of an amide group.

### Nucleotides

Methods described herein involve polymerization of nucleotides via phosphoramidate or thiophosphoramidate linkages using a DNA polymerase variant. Any suitable nucleotide can be used in methods described herein. Nucleotides can be modified or unmodified. Nucleotides can be naturally occurring or synthetic.

For example, the nucleotide can be a 3′-amino-2′,3′-dideoxyribonucleotide 5′-triphosphate (nNTP). Examples of 3′-amino-2′,3′-dideoxyribonucleotide 5′-triphosphate (nNTPs) include 3′-amino-2′,3′-dideoxyadenosine 5′-triphosphate (nATP), 3′-amino-2′,3′-dideoxycytidine 5′-triphosphate (nCTP), 3′-amino-2′,3′-dideoxyguanosine 5′-triphosphate (nGTP), 3′-amino-2′,3′-deoxythymidine 5′-triphosphate (nTTP).

In another example, the nucleotide can be a deoxynucleoside triphosphate (dNTP). Examples of deoxynucleoside triphosphates (dNTPs) include 2′-deoxyadenosine 5′-triphosphate (dATP), 2′-deoxycytidine 5′-triphosphate (dCTP), 2′-deoxyguanosine 5′-triphosphate (dGTP), 2′-deoxythymidine 5′-triphosphate (dTTP).

In yet another example, the nucleotide can be a α-P-thio substituted nucleotide. Examples of α-P-thio substituted nucleosides include 2′-deoxynucleoside 5′-(α-P-thio)triphosphates (dNTPαS), such as 2′-deoxyadenosine 5′-(α-P-thio)triphosphates (dATPαS), 2′-deoxycytidine 5′-(α-P-thio)triphosphates (dCTPαS), 2′-deoxyguanosine 5′-(α-P-thio)triphosphates (dGTPαS), and 2′-deoxythymidine 5′-(α-P-thio)triphosphates (dTTPαS).

In yet another example, the nucleotide can be a nucleoside triphosphate (NTP). Examples of nucleoside triphosphates (NTPs) include adenosine 5′-triphosphate (ATP), cytidine 5′-triphosphate (CTP), guanosine 5′-triphosphate (GTP), and uridine 5′-triphosphate (UTP).

Nucleotides, oligonucleotides, and polynucleotides disclosed herein can comprise any suitable modified nucleotide such as those known in the art. Modified nucleotides include, but are not limited to, nucleotides comprising a backbone modification, a base modification, and/or a sugar modification. Non-limiting examples of backbone modifications include phosphorothioate modifications, methylphosphonate modification, phosphoramidate modifications, and locked nucleic acid (LNA) backbone modifications. Non-limiting examples of base modifications include substituted purines and pyrimidines. Non-limiting examples of sugar modifications include 2′-O-alkylated or 2′-fluorinated ribose and arabinose. Other such modifications are well known to those of skill in the art.

### 3′-Amino Terminated Primers

Methods described herein involve extension of a 3′-amino terminated primer by a DNA polymerase variant to produce oligonucleotides or polynucleotides comprising phosphoramidate-linked nucleotides. The 3′-amino terminated primer can comprise ribonucleotides, deoxyribonucleotides, or a combination thereof.

Nucleotides in the 3′-amino terminated primer can be linked by any type of linkage. Non-limiting examples of linked nucleotides in a 3′-amino terminated primer include phosphodiester-linked nucleotides, phosphoramidate-linked nucleotides, phosphorothioate-linked nucleotides, and thiophosphoramidate-linked nucleotides.

Nucleotides in the 3′-amino terminated primer can be linked by a single type of linkage or by different types of linkages. As such, the 3′-amino terminated primer can be referred to as comprising one type of linkage or mixed types of linkages. For example, when the 3′-amino terminated primer comprises one type of linkage, the 3′-amino terminated primer can comprise phosphodiester-linked nucleotides or phosphoramidate-linked nucleotides. In another example, when the 3′-amino terminated primer comprises multiple types of linkages, the 3′-amino terminated primer can comprise phosphodiester-linked nucleotides and phosphoramidate-linked nucleotides.

The 3′-amino terminated primer can comprise any suitable modified nucleotide such as those known in the art. Modified nucleotides include, but are not limited to, nucleotides comprising a backbone modification, a base modification, and/or a sugar modification. Non-limiting examples of backbone modifications include phosphorothioate modifications, methylphosphonate modification, phosphoramidate modifications, and locked nucleic acid (LNA) backbone modifications. Non-limiting examples of base modifications include substituted purines and pyrimidines. Non-limiting examples of sugar modifications include 2′-O-alkylated or 2′-fluorinated ribose and arabinose. Other such modifications are well known to those of skill in the art.

Any length 3′-amino terminated primer can be used in methods described herein. In some embodiments, the 3′-amino terminated primer is 5 to 200 nucleotides in length. In some embodiments, the is 3′-amino terminated primer is 25 to 200 nucleotides, 50 to 200 nucleotides, 75 to 200 nucleotides, 100 to 200 nucleotides, 125 to 200 nucleotides, 150 to 200 nucleotides, 175 to 200 nucleotides, 5 to 175 nucleotides, 5 to 150 nucleotides, 5 to 125 nucleotides, 5 to 100 nucleotides, 5 to 75 nucleotides, 5 to 50 nucleotides, or 5 to 25 nucleotides in length.

In some embodiments, the 3′-amino terminated primer is 10 to 50 nucleotides, 15 to 50 nucleotides, 20 to 50 nucleotides, 25 to 50 nucleotides, 30 to 50 nucleotides, 35 to 50 nucleotides, 40 to 50 nucleotides, 45 to 50 nucleotides, 5 to 45 nucleotides, 5 to 40 nucleotides, 5 to 35 nucleotides, 5 to 30 nucleotides, 5 to 25 nucleotides, 5 to 20 nucleotides, 5 to 15 nucleotides, or 5 to 10 nucleotides in length.

The 3′-amino terminated primer can comprise any number of consecutive phosphoramidate-linked nucleotides. In some embodiments, the 3′-amino terminated primer comprises at least 5, at least 10, at least 15, at least 20, at least 25, at least 30, at least 35, at least 40, at least 45, or at least 50 consecutive phosphoramidate-linked nucleotides. In some embodiments, each nucleotide in the 3′-amino terminated primer is phosphoramidate-linked.

The 3′-amino group of the 3′-amino terminated primer can be incorporated into a ribonucleotide or a deoxyribonucleotide. Accordingly, the 3′-amino terminated primer can comprise a 3′-amino terminal ribonucleotide or a 3′-amino terminal deoxyribonucleotide.

In some examples, the 3′-amino residue is incorporated into ribonucleotide, and the 3′-amino terminal ribonucleotide can be selected from the group consisting of 3′-amino-adenosine, 3′-amino-guanosine, 3′-amino-cytidine, and 3′-amino-uridine.

In other examples, the 3′-amino residue is incorporated into deoxyribonucleotide, and 3′-amino terminal dideoxynucleotide selected from the group consisting of 3′-amino-2′,3′-dideoxyadenosine (nA), 3′-amino-2′,3′-dideoxythymidine (nT), 3′-amino-2′,3′-dideoxycytidine (nC), 3′-amino-2′,3′-dideoxyguanosine (nG), or 3′-amino-2′,3′-dideoxyuridine (nU).

The 3′-amino terminated primer can be unlabeled or labeled. The 3′-amino terminated primer can be labeled with any suitable label, which can be any suitable chemical or molecule (e.g., a non-nucleotide molecule). Examples of labels include, but are not limited to, fluorescent dyes (e.g., fluorophores), affinity tags (e.g., biotin), luminescent agents, electron-dense reagents, enzymes (e.g., luciferase), isotopes (e.g., ³²P), haptens, lipids (e.g., lipid anchors), sugars (e.g., N-acetylgalactosamine (GalNAc)), linkers, small molecules, and proteins. The 3′-amino terminated primer can be labeled using any method known in the art (e.g., click chemistry).

In some embodiments, the 3′-amino terminated primer comprises a detectable label, which refers to any molecule that is capable of releasing a detectable signal, either directly or indirectly. Any detectable label known in the art (e.g., a fluorescent label or a radioactive label) can be incorporated into the 3′-amino terminated primer. For example, the 3′-amino terminated primer can comprise a detectable label such as a fluorescent label (e.g., fluorescein, Cy3). The detectable label can be attached to any nucleotide in the 3′-amino terminated primer. In some embodiments, the 3′-amino terminated primer comprises a 5′-detectable label.

The 3′-amino terminated primer to be used in methods described herein can be prepared by any known method, e.g., synthetic methods, enzymatic methods, cellular methods, or a combination of any of these.

### DNA Templates

Methods provided herein produce oligonucleotides or polynucleotides comprising phosphoramidate-linked nucleotides in a DNA template-directed manner. The DNA template for use in such methods comprises a sequence complementary to a 3′-amino terminated primer and a nucleic acid sequence of interest. In some embodiments, the DNA template comprises, from 3′ to 5′, a sequence complementary to a 3′-amino terminated primer and a nucleic acid sequence of interest.

Any suitable nucleic acid sequence of interest can be used in a DNA template described herein. The nucleic acid sequence of interest can vary depending on the downstream use of the oligonucleotide or the polynucleotide thus produced. For example, when the oligonucleotide or the polynucleotide can be used in gene editing, the nucleic acid sequence of the oligonucleotide or the polynucleotide can be complementary to the target gene.

DNA templates for use in methods described herein can be linear, e.g., linear DNA templates generated by polymerase chain reaction (PCR), chemical synthesis, or other means known in the art. In some embodiments, the template can be circular, e.g., provided in a vector such as a plasmid.

The DNA template can comprise one type of linkage or multiple types of linkages. For example, when the DNA template comprises one type of linkage, the DNA template can comprise phosphodiester-linked nucleotides or phosphoramidate-linked nucleotides. In another example, when the DNA template comprises multiple types of linkages, the DNA template can comprise phosphodiester-linked nucleotides and phosphoramidate-linked nucleotides.

### II. Methods for Producing Oligonucleotides or Polynucleotides Comprising Phosphoramidate-Linked or Thiophosphoramidate-Linked Nucleotides

Aspects of the present disclosure provide methods for producing oligonucleotides or polynucleotides comprising phosphoramidate-linked (NP-linked) nucleotides or thiophosphoramidate-linked (NPS-linked) nucleotides. Such methods involve incubating a sample comprising a DNA polymerase variant, a divalent metal ion cofactor and/or a trivalent metal ion cofactor, 2′,3′-dideoxyribonucleoside 5′-triphosphates (nNTPs) or a nucleoside α-thiotriphosphate derivative, a 3′-amino terminated primer, and a DNA template in a pH-buffered aqueous solution, each of which are disclosed herein.

To produce an oligonucleotide or a polynucleotide comprising phosphoramidate-linked nucleotides or thiophosphoramidate-linked nucleotides, a sample is incubated under conditions sufficient for the DNA polymerase variant to produce the oligonucleotide or the polynucleotide comprising NP-linked nucleotides or PS-linked nucleotides. Such conditions include, but are not limited to, incubating the sample for a suitable period of time at a suitable temperature and a suitable pH.

Methods described herein encompass incubating a sample for any period of time sufficient for the DNA polymerase variant to produce an oligonucleotide or a polynucleotide comprising phosphoramidate-linked nucleotides or thiophosphoramidate-linked nucleotides. In some embodiments, the sample is incubated for 0.5 to 48 hours. In some embodiments, the sample is incubated for 0.5 to 48 hours, 1 to 48 hours, 2 to 48 hours, 3 to 48 hours, 4 to 48 hours, 5 to 48 hours, 6 to 48 hours, 12 to 48 hours, 24 to 48 hours, 36 to 48 hours, 0.5 to 36 hours, 0.5 to 24 hours, 0.5 to 12 hours, 0.5 to 6 hours, 0.5 to 5 hours, 0.5 to 4 hours, 0.5 to 3 hours, 0.5 to 2 hours, or 0.5 to 1 hours. In some embodiments, the sample is incubated for 0.5 hour, 1 hour, 2 hours, 3 hours, 4 hours, 5 hours, 6 hours, 12 hours, 24 hours, 36 hours, 48 hours, or more.

Methods described herein encompass incubating a sample at any temperature sufficient for the DNA polymerase variant to produce an oligonucleotide or a polynucleotide comprising phosphoramidate-linked nucleotides or thiophosphoramidate-linked nucleotides.

In some embodiments, the sample is incubated at a temperature of 25 to 37° C. , e.g., at a temperature of 25° C., 26° C., 27° C., 28° C., 29° C., 30° C., 31° C., 32° C., 33° C., 34° C., 35° C., 36° C., or 37° C. In some embodiments, the sample is incubated at a temperature of 37° C.

In some embodiments, the sample is incubated at a temperature of 50 to 65° C., e.g., at a temperature of 50° C., 51° C., 52° C., 53° C., 54° C., 55° C., 55° C., 57° C., 58° C., 59° C., 60° C., 61° C., 62° C., 63° C., 64° C., or 65° C. In some embodiments, the sample is incubated at a temperature of 55° C.

In some embodiments, the sample is incubated at a temperature of 40 to 50° C., e.g., at a temperature of 40° C., 41° C., 42° C., 43° C., 44° C., 45° C., 46° C., 47° C., 48° C., 49° C., or 50° C. In some embodiments, the sample is incubated at a temperature of 45° C.

Methods described herein encompass incubating a sample at any pH sufficient for the DNA polymerase variant to produce an oligonucleotide or a polynucleotide comprising phosphoramidate-linked nucleotides. In some embodiments, the sample is incubated at a pH of 7 to 10, 8 to 10, 9 to 10, 7 to 9, or 7 to 8. In some embodiments, the sample is incubated at a pH of 8.6 to 9.2, 8.7 to 9.2, 8.8 to 9.2, 8.9 to 9.2, 9.0 to 9.2, 9.1 to 9.2, 8.6 to 9.1, 8.6 to 9.0, 8.6 to 8.9, 8.6 to 8.8, or 8.6 to 8.7. In some embodiments, the sample is incubated at a pH of 8.8.

In some embodiments, buffer is added to a sample to achieve a particular pH and/or salt concentration. Examples of buffers include, but are not limited to, phosphate buffer, Tris buffer, MOPS buffer, HEPES buffer, EPPS buffer, and POPSO buffer.

Any suitable trivalent metal ion cofactor can be used in methods described herein. Accordingly, the sample can comprise any suitable trivalent metal ion cofactor sufficient for the DNA polymerase variant to produce an oligonucleotide or a polynucleotide comprising phosphoramidate-linked nucleotides. In some embodiments, the sample comprises Sc³⁺, Y³⁺, Lu³⁺, In³⁺, Eu³⁺, Gd³⁺, Yb³⁺, or Er³⁺ or a combination thereof. In some embodiments, the sample comprises Sc³⁺.

The sample can comprise any suitable concentration of a trivalent metal ion cofactor. In some embodiments, the sample comprises a trivalent metal ion cofactor at a concentration of 0.1 to 50 mM. In some embodiments, the sample comprises a trivalent metal ion cofactor at a concentration of 0.1 to 5 mM, 5 to 50 mM, 10 to 50 mM, 15 to 50 mM, 20 to 50 mM, 25 to 50 mM, 30 to 50 mM, 35 to 50 mM, 40 to 50 mM, 45 to 50 mM, 5 to 45 mM, 5 to 40 mM, 5 to 35 mM, 5 to 30 mM, 5 to 25 mM, 5 to 20 mM, 5 to 15 mM, or 5 to 10 mM.

Any amount of metal chelating agent suitable for chelating a trivalent metal ion cofactor can be used in methods described herein. For example, in methods described herein, a sample can comprise a metal chelating agent at a concentration of about 0.1 to about 50 mM, e.g., at a concentration of 0.1 to 5 mM, 5 to 50 mM, 10 to 50 mM, 15 to 50 mM, 20 to 50 mM, 25 to 50 mM, 30 to 50 mM, 35 to 50 mM, 40 to 50 mM, 45 to 50 mM, 5 to 45 mM, 5 to 40 mM, 5 to 35 mM, 5 to 30 mM, 5 to 25 mM, 5 to 20 mM, 5 to 15 mM, or 5 to 10 mM. In another example, in methods described herein, a sample can comprise a metal chelating agent and a trivalent metal ion cofactor in a stoichiometry of about 0.5:1, about 1:1, or about 1:2 (trivalent metal ion cofactor:metal chelating agent).

Any DNA polymerase variant, such as those described herein, can be used in methods provided herein. Accordingly, the sample comprises any DNA polymerase variant described herein. In some embodiments, the sample can comprise a DNA polymerase variant, which comprises an amino acid sequence that is at least 70%, 80%, 90%, or 95% identical to SEQ ID NO: 1. In some embodiments, the sample comprises a DNA polymerase variant set forth in SEQ ID NO: 1.

Methods described herein encompass incubating a sample comprising any amount of a DNA polymerase variant sufficient for the DNA polymerase variant to produce an oligonucleotide or a polynucleotide comprising phosphoramidate-linked nucleotides or thiophosphoramidate-linked nucleotides. For example, the sample can comprise the DNA polymerase variant at a concentration of 0.001 to 50 μM (e.g., at a concentration of 0.001 μM, 0.01 μM, 0.1 μM, 0.5 μM, 1 μM, 5 M, 10 μM, or 50 μM).

DNA polymerase variants in any suitable form can be used in methods described herein. For example, the DNA polymerase variant can be provided as a cell lysate from cells that express the DNA polymerase variant. In another example, the DNA polymerase variant can be provided as a recombinant protein purified from cells that express the DNA polymerase variant. In yet another example, the DNA polymerase variant can be provided as nucleic acids encoding the DNA polymerase variant.

Methods described herein involve template-directed polymerization of 3′-amino-2′,3′-dideoxyribonucleoside 5′-triphosphates (nNTPs) to produce oligonucleotides or polynucleotides comprising phosphoramidate-linked nucleotides. Accordingly, methods described herein comprise incubating a sample comprising nNTPs. In some embodiments, the sample comprises 3′-amino-2′,3′-dideoxyadenosine 5′-triphosphate (nATP), 3′-amino-2′,3′-dideoxycytidine 5′-triphosphate (nCTP), 3′-amino-2′,3′-dideoxyguanosine 5′-triphosphate (nGTP), 3′-amino-2′,3′-deoxythymidine 5′-triphosphate (nTTP), or a combination thereof.

Methods described herein involve template-directed polymerization of α-P-thio substituted nucleosides to produce oligonucleotides or polynucleotides comprising thiophosphoramidate-linked nucleotides. Accordingly, methods described herein comprise incubating a sample comprising 2′-deoxynucleoside 5′-(α-P-thio)triphosphates (dNTPαS). In some embodiments, the sample comprises 2′-deoxyadenosine 5′-(α-P-thio)triphosphates (dATPαS), 2′-deoxycytidine 5′-(α-P-thio)triphosphates (dCTPαS), 2′-deoxyguanosine 5′-(α-P-thio)triphosphates (dGTPαS), 2′-deoxythymidine 5′-(α-P-thio)triphosphates (dTTPαS), or a combination thereof.

Methods described herein can produce oligonucleotides or polynucleotides comprising mixed linkages (e.g., phosphoramidate-linked nucleotides and phosphodiester-linked nucleotides or thiophosphoramidate-linked nucleotides and phosphodiester-linked nucleotides) using nucleotides comprising a 3′-hydroxyl group and lacking a 3′-amino modification. For example, in such methods, the sample can comprise nNTPs (e.g., dNTPαS) and deoxynucleoside triphosphates (dNTPs). In some embodiments, the sample can comprise 2′-deoxyadenosine 5′-triphosphate (dATP), 2′-deoxycytidine 5′-triphosphate (dCTP), 2′-deoxyguanosine 5′-triphosphate (dGTP), 2′-deoxythymidine 5′-triphosphate (dTTP), or a combination thereof. In another example, the sample can comprise nNTPs and nucleoside triphosphates (NTP). In some embodiments, the sample can comprise adenosine 5′-triphosphate (ATP), cytidine 5′-triphosphate (CTP), guanosine 5′-triphosphate (GTP), and uridine 5′-triphosphate (UTP), or a combination thereof.

Any suitable 3′-amino terminated primer, such as those described herein, can be used in methods provided herein. Accordingly, methods described herein comprise incubating a sample comprising any 3′-amino terminated primer. In some embodiments, the sample can comprise a 3′-amino terminated primer comprising ribonucleotides and/or deoxyribonucleotides. In some embodiments, the sample can comprise a 3′-amino terminated primer comprising a 5′-detectable label.

Any suitable amount of a 3′-amino terminated primer can be used in methods described herein. For example, the sample can comprise a 3′-amino terminated primer at a concentration of 0.1 to 10 μM. In some embodiments, the sample can comprise a 3′-amino terminated primer at a concentration of 0.25 to 10 μM, 0.5 to 10 μM, 1 to 10 μM, 2.5 to 10 μM, 5 to 10 μM, 7.5 to 10 μM, 0.1 to 7.5 μM, 0.1 to 5 μM, 0.1 to 2.5 μM, 0.1 to 1 μM, 0.1 to 0.5 μM, or 0.1 to 0.25 μM.

Any suitable DNA template, such as those described herein, can be used in methods provided herein. Accordingly, methods described herein comprise incubating a sample comprising a DNA template. In some embodiments, the sample comprises a DNA template comprising, from 3′ to 5′, a sequence complementary to a 3′-amino terminated primer and a nucleic acid sequence of interest.

Any suitable amount of a DNA template can be used in methods described herein. For example, the sample can comprise a DNA template at a concentration of 0.1 to 10 μM. In some embodiments, the sample can comprise a DNA template at a concentration of 0.25 to 10 μM, 0.5 to 10 M, 1 to 10 μM, 2.5 to 10 μM, 5 to 10 μM, 7.5 to 10 μM, 0.1 to 7.5 μM, 0.1 to 5 μM, 0.1 to 2.5 μM, 0.1 to 1 μM, 0.1 to 0.5 μM, or 0.1 to 0.25 μM.

Methods described herein encompass incubating a sample of any suitable volume. For example, the sample can comprise a volume of 0.01 mL, 0.1 mL, 1 mL, 100 mL, 1 L, or more. Depending on the size of the sample, the amount of the oligonucleotide or the polynucleotide produced according to methods described herein can be 1 ng to 1 mg or more (e.g., 1 ng, 10 ng, 100 ng, 1 μg, 10 μg, 100 μg, 1 mg, or more).

### III. Oligonucleotides or Polynucleotides Comprising Phosphoramidate-Linked or Thiophosphoramidate-Linked Nucleotides

Aspects of the present disclosure provide an oligonucleotide or the polynucleotide comprising phosphoramidate-linked (NP-linked) nucleotides or thiophosphoramidate-linked (NPS-linked) nucleotides produced according to methods described herein.

Oligonucleotides or polynucleotides produced according to methods described herein can be various lengths. For example, the oligonucleotide or the polynucleotide can be at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 500, at least 750, at least 1000, or more nucleotides in length.

In some embodiments, the oligonucleotide or the polynucleotide is 25 to 1000 nucleotides in length. In some embodiments, the oligonucleotide or the polynucleotide is 25 to 750 nucleotides, 25 to 500 nucleotides, 25 to 250 nucleotides, 25 to 100 nucleotides, 50 to 1000 nucleotides, 100 to 1000 nucleotides, 250 to 1000 nucleotides, 500 to 1000 nucleotides, or 750 to 1000 nucleotides.

In some embodiments, the oligonucleotide or the polynucleotide is 25 to 250 nucleotides in length. In some embodiments, the oligonucleotide or the polynucleotide is 25 to 225 nucleotides, 25 to 200 nucleotides, 25 to 175 nucleotides, 25 to 150 nucleotides, 25 to 125 nucleotides, 25 to 100 nucleotides, 25 to 75 nucleotides, 25 to 50 nucleotides, 50 to 250 nucleotides, 75 to 250 nucleotides, 100 to 250 nucleotides, 125 to 250 nucleotides, 150 to 250 nucleotides, 175 to 250 nucleotides, 200 to 250 nucleotides, or 225 to 250 nucleotides.

In some embodiments, the oligonucleotide or the polynucleotide is 25 to 150 nucleotides in length. In some embodiments, the oligonucleotide or the polynucleotide is 25 to 125 nucleotides, 25 to 100 nucleotides, 25 to 75 nucleotides, 25 to 50 nucleotides, 50 to 150 nucleotides, 75 to 150 nucleotides, 100 to 150 nucleotides, or 125 to 150 nucleotides.

In some embodiments, the oligonucleotide or the polynucleotide is 25 to 100 nucleotides in length. In some embodiments, the oligonucleotide or the polynucleotide is 25 to 95 nucleotides, 25 to 90 nucleotides, 25 to 85 nucleotides, 25 to 80 nucleotides, 25 to 75 nucleotides, 25 to 70 nucleotides, 25 to 65 nucleotides, 25 to 60 nucleotides, 25 to 55 nucleotides, 25 to 50 nucleotides, 25 to 45 nucleotides, 25 to 40 nucleotides, 25 to 35 nucleotides, or 25 to 30 nucleotides in length.

In some embodiments, the oligonucleotide or the polynucleotide is 30 to 100 nucleotides, 35 to 100 nucleotides, 40 to 100 nucleotides, 45 to 100 nucleotides, 50 to 100 nucleotides, 55 to 100 nucleotides, 60 to 100 nucleotides, 65 to 100 nucleotides, 70 to 100 nucleotides, 75 to 100 nucleotides, 80 to 100 nucleotides, 85 to 100 nucleotides, 90 to 100 nucleotides, or 95 to 100 nucleotides in length.

Nucleotides in the oligonucleotide or the polynucleotide produced according to methods described herein can be ribonucleotides and/or deoxyribonucleotides. An oligonucleotide comprising ribonucleotides and deoxyribonucleotides can be referred to as a mixed or chimeric oligonucleotide. In general, a mixed oligonucleotide refers to an oligonucleotide comprising a mix of structural components such as a mix of nucleotides (e.g., ribonucleotides and deoxyribonucleotides) and/or an oligonucleotide comprising two or more kinds of backbone or internucleotide linkages (e.g., phosphoramidate linkages and phosphodiester linkages).

Nucleotides in the oligonucleotide or the polynucleotide can comprise any modification known in the art such as those described herein. In some embodiments, one or more nucleotides in the oligonucleotide or the polynucleotide can comprise a backbone modification, which can reduce nuclease-mediated degradation of the oligonucleotide. For example, one or more of the nucleotides in the oligonucleotide or the polynucleotide can comprise a locked nucleic acid (LNA) modification in which the nucleotide comprises a modified sugar residue with an additional 2′-C,4′-C-oxymethylene linker that confines the ribose ring to the 3′-endo conformation.

Alternatively, or in addition to, one or more nucleotides in the oligonucleotide or the polynucleotide can comprise a modification of the base or the sugar moieties. For example, one or more nucleotides in the oligonucleotide or the polynucleotide can comprise arabinose instead of ribose. In another example, one or more nucleotides in the oligonucleotide or the polynucleotide can comprise a substituted base such as a substituted pyrimidine (e.g., 5-methylcytosine) or isomer (e.g., pseudouridine).

At least a portion of the nucleotides in the oligonucleotide or the polynucleotide can be linked by phosphoramidate linkages. In some examples, each nucleotide in the oligonucleotide or the polynucleotide is phosphoramidate-linked. In other examples, a portion of the nucleotides in the oligonucleotide or the polynucleotide can be phosphoramidate-linked and a portion of the nucleotides in the oligonucleotide or the polynucleotide can be linked via another type of linkage (e.g., phosphodiester linkage, phosphorothioate linkage). In such instances, the oligonucleotide or the polynucleotide can be referred to as having mixed linkages.

In some embodiments, the oligonucleotide or the polynucleotide comprises a plurality of phosphoramidate-linked nucleotides. In some embodiments, the oligonucleotide or the polynucleotide comprises at least 5, at least 10, at least 15, at least 20, at least 25, at least 30, at least 35, at least 40, at least 50, at least 60, at least 70, at least 80, at least 90, at least 100, or more consecutive phosphoramidate-linked nucleotides. In some embodiments, each nucleotide in the oligonucleotide or the polynucleotide is phosphoramidate-linked. In some embodiments, the oligonucleotide or the polynucleotide incorporates at least one thiophosphoramidate-linked nucleotide.

In some embodiments, the oligonucleotide or the polynucleotide comprises a plurality of thiophosphoramidate-linked nucleotides. In some embodiments, the oligonucleotide or the polynucleotide comprises at least 5, at least 10, at least 15, at least 20, at least 25, at least 30, at least 35, at least 40, at least 50, at least 60, at least 70, at least 80, at least 90, at least 100, or more consecutive thiophosphoramidate-linked nucleotides. In some embodiments, each nucleotide in the oligonucleotide or the polynucleotide is thiophosphoramidate-linked.

At least one of the thiophosphoramidate-linkages in the oligonucleotide or the polynucleotide can be stereopure. In such instances, the stereochemistry at the thiophosphoramidate-linkage is defined as Sp or Rp. In some embodiments, each of the thiophosphoramidate-linked nucleotides in the oligonucleotide or the polynucleotide are stereopure. In such instances, the oligonucleotide or the polynucleotide can comprise a common length, a common base sequence, a common backbone linkage, and a common backbone chiral center.

Oligonucleotides or polynucleotides described herein can include nucleotides linked by a single type of linkage or by different types of linkages. As such, the oligonucleotide or the polynucleotide can be referred to as comprising one type of linkage or mixed types of linkages. For example, when the oligonucleotide or the polynucleotide comprises one type of linkage, the oligonucleotide or the polynucleotide can include phosphodiester-linked nucleotides, phosphoramidate-linked nucleotides, phosphorothioate-linked nucleotides, or thiophosphoramidate-linked nucleotides. In another example, when the oligonucleotide or the polynucleotide comprises multiple types of linkages, the oligonucleotide or the polynucleotide can include any combination of phosphodiester-linked nucleotides, phosphoramidate-linked nucleotides, phosphorothioate-linked nucleotides, and thiophosphoramidate-linked nucleotides.

The oligonucleotide or polynucleotide produced by methods described herein can be any form. Examples include, but are not limited to, aptamers, antisense oligonucleotides (e.g., antisense RNA, antisense DNA, DNA/RNA heteroduplex oligonucleotides, splice-switching oligonucleotides), guide RNA (gRNA), messenger RNA (mRNA), micro RNA (miRNA), small interfering RNA (siRNA), and single-stranded DNA.

In some instances, two different oligonucleotides or polynucleotides having complementary sequences can be produced according to methods described herein, and the resulting oligonucleotides or polynucleotides can be used to form a double-stranded oligonucleotide or polynucleotide.

In some instances, two different polynucleotides having complementary sequences can be produced according to methods described herein, and the resulting polynucleotides can be used to form a double-stranded polynucleotide.

### IV. Applications of Oligonucleotides or Polynucleotides Comprising Phosphoramidate-Linked or Thiophosphoramidate-Linked Nucleotides

Any of the oligonucleotides and polynucleotides described herein can be used in any suitable application in which oligonucleotides and polynucleotides are utilized. One of ordinary skill in the art will readily recognize that the present disclosure is not limited to particular use but is applicable to any situation in which the use of oligonucleotides or polynucleotides is desirable. For example, oligonucleotides or polynucleotides disclosed herein can be used in microarray technologies, fluorescence in situ hybridization (FISH) technologies, antisense applications, sequencing, and gene editing technologies (e.g., CRISPR-Cas9 gene editing technologies). In another example, oligonucleotides or polynucleotides disclosed herein can be used as agents for modulating a cellular process and/or a cellular machinery, including but not limited to, transcription, translation, immune responses, and epigenetics. Accordingly, any of the oligonucleotides or polynucleotides disclosed herein can be used for therapeutic, diagnostic, agricultural, and/or research purposes.

In such instances, an oligonucleotide or a polynucleotide described herein can comprise a sufficient degree of complementarity to a target element (e.g., a target gene, target mRNA, target regulatory element) to reduce expression of the target element or other activity at the target element. A sufficient degree of complementarity ensures that the oligonucleotide or the polynucleotide specifically binds to the target sequence and avoids non-specific binding of the oligonucleotide or the polynucleotide to non-target sequences under conditions in which specific binding is desired, e.g., under physiological conditions in the case of in vivo assays or therapeutic treatment, or in the case of in vitro assays, under conditions in which the assays are performed.

Any method known in the art can be used to deliver any of the oligonucleotides or the polynucleotides described herein to a cell, a tissue, or an organism. For example, the oligonucleotide or the polynucleotide can be delivered by injection (e.g., microinjection), electroporation, and liposome-mediated transfection.

When used in a therapeutic application, an oligonucleotide or a polynucleotide described herein can be administered to a subject. In such instances, the oligonucleotide or the polynucleotide can be mixed with a pharmaceutically acceptable carrier (excipient) to form a pharmaceutical composition for use in treating a target disease. “Acceptable” means that the carrier must be compatible with the oligonucleotide or the polynucleotide and not deleterious to the subject to be treated. Pharmaceutically acceptable excipients (carriers) including buffers are well known in the art. See, e.g., Remington: The Science and Practice of Pharmacy 20^{th} Ed. (2000) Lippincott Williams and Wilkins, Ed. K. E. Hoover.

The present disclosure also provides a kit for producing an oligonucleotide or a polynucleotide comprising phosphoramidate-linked nucleotides or thiophosphoramidate-linked nucleotides comprising one or more containers of a DNA polymerase variant described herein, a trivalent metal ion cofactor, and optionally with instructions for using the DNA polymerase variant and the trivalent metal ion cofactor for producing the oligonucleotide.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Materials and Methods: Examples 1-4

The following materials and methods were used in the Examples 1-4 set forth herein.

Materials. 3′-Deoxyribonucleotide 5′-triphosphates were obtained from New England Biolabs. 3′-Amino-2′,3′-didexoyribonucleotide 5′-triphosphates were obtained from Trilink Biotechnologies. DNA oligonucleotides were purchased from IDT. Oligonucleotides incorporating a 3′-amino residue were synthesized and purified by standard methods, using phosphoramidites obtained from Chemgenes or as recently reported (Lelyveld et al., DNA polymerase activity on synthetic N3′→P5′ phosphoramidate DNA templates. Nucleic Acids Res. 47, 8941-8949 (2019)) and using reagents purchased from Glen Research on an Expedite 8909 synthesizer. Premixed reagent solutions for crystallization were purchased from Hampton Research. Recombinant lysozyme and Bugbuster lysis reagent were purchased from EMD. Ni-NTA Superflow affinity resin was purchased from Qiagen. All other buffers and reagents were prepared from high purity chemicals obtained from Sigma Sigma-Aldrich, using chemicals with trace metals analysis where available. Competent cells were obtained from New England Biolabs.

Recombinant protein preparation. A bacterial expression plasmid carrying the codon-optimized open reading frame for the DNA polymerase I large fragment (amino acids 297-877) from Bst (BF) fused at its N-terminus to a His6 tag and an HRV 3C protease signal sequence, was produced by standard methods. BF expression was placed under the control of a lacO-regulated T5 promoter based on the expression system from pQE-30. The plasmid backbone carries a kanamycin resistance cassette and lacI derived from pET-28. The resulting plasmid, pVSL5, was used to transform DH5α cells and selected on LB plates containing kanamycin. For protein expression, starter cultures were inoculated from a single colony and grown overnight in LB+1% glucose and kanamycin at 30 μg/mL at 30° C. The overnight culture was then inoculated 1:100 into a shake flask containing LB and antibiotic, and the culture was expanded at 37° C. with 225 rpm shaking (e.g., with 0.5 L medium in a 2 L flask). When the optical density at 600 nm reached ˜0.6-0.8 in a 1 cm path length cuvette, expression of BF was induced by addition of 1 mM IPTG, and induction was allowed to proceed for 2 hr at 37° C. Cells were harvested by centrifugation at 4° C., and the resulting cell pellet was typically stored at −80° C. prior to purification. Thawed pellets were lysed in 1X BugBuster, adjusted to pH ˜8.5 by addition of KOH, containing 0.001X EDTA-free Protease Inhibitor Cocktail Set III (Calbiochem) and lysozyme. The lysate was heat treated in a water bath at 50° C. for 20 min, and its viscosity was reduced by probe sonication at 0° C. The lysate was then clarified by centrifugation at 16000 g for 30 min at 4° C. The polyhistidine-tagged protein was purified by batch binding to Ni-NTA beads that had been pre-equilibrated in 1X BugBuster. The resin was washed with 1 column volume (CV) of BugBuster, followed by 10 CV of 20 mM sodium phosphate, pH 7.6, 0.5 M NaCl, 12.5 mM imidazole. An additional wash with a higher salt buffer, otherwise as above except containing 1 M NaCl, reduced nonspecific binding. The protein was eluted with 20 mM sodium phosphate, pH 7.6, 0.5 M NaCl, 250 mM imidazole, and DTT was added to the resulting fractions up to 2 mM. The protein was concentrated using centrifugal MWCO filtration devices (Millipore), desalted into a 2X storage buffer using a disposable size exclusion column (NAP25, GE Amersham) following the manufacturer's protocol, and then reconcentrated. The 2X storage buffer contained 20 mM Tris-Cl, pH 7.6, 100 mM KCl, 2 mM DTT, 0.2 mM EDTA, and 0.2% Triton X-100. The resulting protein preparation was diluted 2-fold by gradual addition of 1 volume of glycerol, then stored at −30° C. To prepare protein for crystallization, elution fractions from the Ni-NTA resin were diluted 1:1 by addition of the 2X storage buffer without Triton X-100, followed by HRV 3C protease for cleavage of the affinity tag. The cleavage reaction proceeded overnight at 4° C., and the His-tagged protease and cleaved peptide were removed by passing the solution over Ni-NTA resin, followed by concentration and desalting into 1X storage buffer, as above except omitting the nonionic surfactant.

Co-crystallizations. Preparations of the wild type or D598A/F710Y double-mutant BF were concentrated to 20 mg/mL. The mutation D598A reportedly disrupts a crystal contact that otherwise precludes crystallization of the closed conformation of the fingers domain (Johnson et al., Processive DNA synthesis observed in a polymerase crystal suggests a mechanism for the prevention of frameshift mutations. Proc. Natl. Acad. Sci. 100, 3895-3900 (2003)). A DNA primer, containing either a 3′ terminal 2′,3′-dideoxycytidine (5′-GCGATCAGddC (SEQ ID NO: 2)) or 3′-amino-2′,3′-dideoxycytidine (5′-GCGATCAGnC (SEQ ID NO: 2)) residue, was prepared and annealed with a partially complementary DNA template oligonucleotide (5′-ACACGCTGATCGCA (SEQ ID NO: 3)) in the presence of either dGTP, nGTP, or dGpNHpp, as indicated. The final concentrations of primer/template duplex and substrate were 0.25 mM and 3 mM, respectively. Protein was added to a final concentration of 10 mg/mL and allowed to complex by incubating at 37° C. for ˜10-20 min or overnight at 4° C. To obtain crystals of the substrate-bound closed conformation, mixtures of the D598A/F710Y double mutant protein containing substrate, primer, and template were screened by mixing 1:1 with crystallization reagent solutions containing 0.1 M Na-MES pH 5.4 or 5.8 buffer, 2 M ammonium sulfate, 2.5% or 5% (v/v) (+/−)-2-methyl-2,4-pentanediol, and 10 mM of MgSO₄, CaCl₂, MnSO₄, or CoCl₂. Structures in the closed conformation with ordered substrate and metal ion were obtained only with calcium, manganese, or cobalt ions. Crystallization proceeded by the sitting-drop vapor diffusion method. Crystals typically appeared within 2-4 days at 18° C. Prior to mounting and freezing, crystals were soaked in a solution of the same reagent mix as for crystallization, except where the buffer was replaced with 0.1 M Na-EPPS, pH 8.8, and the substrate concentration was 0.5 mM. Soak time was screened from ˜ 2 min-90 min at 20° C. For the closed conformation structure containing Ca²⁺ and dGpNHpp (6UR9), the crystal was formed at pH 5.4 with 2.5% MPD, and the soak time at pH 8.8 was ˜20 min. For the closed conformation structure containing Mn²⁺ and nGTP (6US5), the crystal was formed at pH 5.4 with 5% MPD, and the soak time was ˜5 min at pH 8.8. Crystals of the wild-type BF complex in the open conformation were prepared similarly, except using 8 mM dGTP and 20 mM MgCl₂ in the protein complex mix, and these crystals were mounted and frozen directly from the mother liquor without any additional soaking. No ordered density corresponding dGTP or Mg²⁺ were observed in these open conformation wild-type structures. The pre-reaction open structure (0 Complex, 6UR4) was crystallized by mixing the protein complex solution 1:1 with 60% v/v Tacsimate, pH 7.0. Tascimate (Hampton Research) is a reagent mix containing 1 M malonic acid, 0.15 M ammonium citrate tribasic, 0.07 M Succinic acid, 0.18 M DL-malic acid, 0.24 M sodium acetate trihydrate, 0.3 M sodium formate, 0.1 M ammonium tartrate dibasic, pH 7.0. The post-reaction translocated open structure (+1 Complex, 6UR2), with incorporated dGTP, was obtained after ˜45 d by mixing the same protein complex solution 1:1 with 0.8 M sodium succinate, pH 7.0. For all crystals, the space group was P2₁2₁2₁, and the structures were solved by molecular replacement.

Primer extension assays. Except where indicated, all extension reactions were performed with 1 μM fluorescein-labeled primer, 1.5 μM template, and ˜1.1 μM purified His6-tagged protein at 55° C. in a reaction buffer composed of 40 mM Tris-HCl, pH 8.8, 1 mM DTT, and a divalent cation as specified, typically 10 mM CaCl₂. Reaction mixtures were equilibrated at the indicated reaction temperature for 1 min and initiated by addition of substrate. Progress was monitored by sampling 1 μL from the reaction manually quenched into 24 μL of chilled 90% formamide, 10 mM EDTA. Quenched samples were denatured at 90° C. for 1 min and cooled to room temperature prior to separation by denaturing polyacrylamide gel electrophoresis on 20% TBE-urea gels. To maintain strand separation for long extension products, as in FIGS. 3F-3H, an unlabeled DNA oligonucleotide complementary to the template was added to the quenching buffer (˜2 μM) prior to denaturation. Typically, 3 or 4 μL of quenched sample was loaded per lane for gels of 0.75-mm thickness. Bands were visualized with a Typhoon laser scanning imager (GE Amersham) and quantified with the manufacturer's software. For acid digestion of extended products, a reaction sample was diluted with 2.5 volumes of 1% acetic acid, 10 mM EDTA in formamide and digested at 75° C. for 45 min prior to gel separation.

Mass spectrometry. Reaction samples were desalted using C18 ZipTips equilibrated with LC-MS grade methanol followed by 2 M triethylammonium acetate, pH 7 (TEAA). The sample was diluted by addition of TEAA buffer to a concentration of 0.2 M and bound to the support. The resin was then washed with at least 100 μL of 20 mM TEAA. The sample was eluted in 50% methanol directly into an HPLC vial insert and dried in a centrifugal vacuum concentrator.

### Materials and Methods: Examples 5-9

The following materials and methods were used in the Examples 5-9 set forth herein.

Materials. 3′-Amino-2′,3′-dideoxynucleoside 5′-triphosphates were purchased from Trilink BioTechnologies. Stereopure 2′-deoxycytidine 5′-O-(1-thiotriphosphate), Sp- or Rp-dCTPαS, were obtained from Biolog Life Science Institute. PAGE purified 3′-amino terminal DNA oligonucleotide primers were prepared using 3′-amino-2′,3′-dideoxynucleoside 5′-phosphoramidites and reverse DNA phosphoramidites from Chemgenes, using oligonucleotide synthesis reagents from Glen Research. DNA template oligonucleotides were synthesized by IDT. Crystallization reagents were obtained from Hampton Research. High purity trivalent rare earth metal salts were obtained from Sigma Aldrich, Alfa Aesar, or Strem Chemicals. In particular, Scandium (III) chloride hexahydrate, 99.9%, was obtained from Strem Chemicals. Tag-cleaved BF polymerase variants F710Y/D598A or D598A protein were prepared essentially as previously described (1), with minor modifications. In particular, DH5αF'Iq cells (New England Biolabs) were used for protein expression for F710Y/D598A from a modified version of the plasmid in (1) incorporating the lacI^{q} promoter mutation. Cell lysis was performed with NEBExpress Lysis Reagent (New England Biolabs) pH-adjusted to ˜8.5 with KOH, otherwise as previously described (2).

Time resolved X-ray crystallography and analysis. The conditions for BF polymerase catalyzed extension of a 3′-amino terminal DNA primer on a DNA template in intact crystals were assessed by bright field microscopy for their ability maintain crystal integrity over the full time course of bond formation, up to 24 hours for BF mutant F710Y/D598A and 48 hours for wild type (D598A) with reaction progress monitored by HPLC of redissolved crystals. The quaternary complex was assembled with protein, primer, template, Ca²⁺ ion, and 2′-deoxyguanosine 5′-triphosphate (dGTP) substrate and crystallized within 1-2 days under mild acidic conditions (pH 5-6) below the pKa of the primer terminal 3′-amino group (˜7.7 as the free aminonucleoside (35)), such that the extension reaction could be initiated simply by pH shift. Crystals were transferred from the mother liquor to a similar soaking solution at pH 8.8, then flash frozen in liquid nitrogen at a range of time points for subsequent data collection. The mother liquor for crystallization was 2 M ammonium sulfate, 0.1 M MES, pH 5.3-6.0, 20 mM CaCl₂, and 5% MPD mixed 1:1 with a protein-nucleic acid complexes prepared with 0.15 mM BF F710Y/D598A or D598A, 0.2 mM DNA template, 0.2 mM 3′-amino terminal DNA primer, and 10 mM dGTP, essentially as in (1). The composition of the soaking solution was 2 M ammonium sulfate, 0.1 M EPPS, pH 8.8, 20 mM CaCl₂, 5% MPD, and 10 mM dGTP. Although we observed two fully assembled complexes in the “closed” conformation in the asymmetric unit under these crystallization conditions, it was found during refinement that ground state B-factors were appreciably different between the two complexes, possibly due to distinctions in crystal packing and associated solvent channels in the crystal. Analyses performed here rely exclusively on data from the complex with lower B-factor (model chains A-C), although equivalent kinetic trends are observable in the second complex as well.

We adopted an analysis method similar to that presented in Samara et al. (36), but with several key modifications. Briefly, we begin with a refined ground state model and then prepare difference maps (Fₒ−F_{c}) from each observed soaked crystal dataset vs. the ground state model, where structure factors have been scaled together using the CCP4 program SCALEIT essentially as described in (36). However, in this work we have not omitted any atoms from the reaction center in ground state model refinement. Since any model bias is time invariant, this approach better maintains consistent scaling for density changes, both positive and negative, across real space maps which would not otherwise be the case for dynamics occurring in unmodeled regions.

Pearson correlation maps were calculated by selecting the real space coordinates of the nascent bond (or any other point of interest) as a reference point and then calculating the pairwise correlation coefficient, r, of difference map density from n distinct Fₒ−F_{c} difference maps across all points on the real space grid (x, y, z),

$$r ( x , y , z ) = \frac{{\sum }_{i}^{n} ( {Δρ}_{ref , i} - \overline{{Δρ}_{ref}} ) ( {Δρ}_{i} ( x , y , z ) - \overline{Δρ ( x , y , z )} )}{\sqrt{{\sum }_{i}^{n} {( {Δρ}_{ref , i} - \overline{{Δρ}_{ref}} )}^{2} {\sum }_{i}^{n} {( {Δρ}_{i} ( x , y , z ) - \overline{Δρ ( x , y , z )} )}^{2}}}$$

where Δ̅σ̅_̅{̅r̅e̅f̅}̅ is the mean difference map density at a reference point, Δ̅ρ̅ is the mean difference map density at lattice point (x, y, z), Δρ_{ref,i} is the density at the reference point in difference map i, and Δρᵢ(x, y, z) is the density at grid point (x, y, z) in difference map i. The resulting correlation maps have r values on the interval [−1,1] and can be contoured for visualization at a desired correlation level to generate coefficient isosurfaces in which, for example, the correlation coefficient is greater than or less than a threshold value. Two-sided p-values were calculated by the pearsonr function of the Python scipy.stats package.

Linear regression “beta” maps were produced by least squares regression to estimate the slope β for the best fit line,

$${Δρ}_{ref} = βΔρ ( x , y , z ) + α ,$$

between observed density differences at a reference point, Δρ_{ref}=[Δρ_{ref,1}, Δρ_{ref,2}, . . . , Δρ_{ref,i}], versus any “query” lattice point, Δρ(x, y, z)=[Δρ₁, Δρ₂, . . . , Δρᵢ], where the parameter α corrects for crystal-invariant (i.e. time-invariant) density differences across real space. Regression maps are then contoured for visualization at a desired β value to yield isosurfaces within which β is greater than or less than a desired threshold value. Regression statistics (e.g. coefficients of determination, R²) or correlation coefficients, r, can also in principle be used for filtering β maps, but no such filtering was performed here. Regression and correlation analyses of the spatiotemporal data are closely related, but they offer complementary information about the dynamics specifically relevant to bond formation without requiring more involved dimensionality reduction.

Similarly, observed first-order rate constant estimates, k_{obs}, were obtained by nonlinear regression of time-varying density differences Δρ at any real space position (x, y, z) using the expression,

$$Δρ ( x , y , z ) = A ( 1 - {e}^{- {k}_{obs} t} ) ,$$

for density differences calculated across maps, Δρ(x, y, z)=[Δρ₁, Δρ₂, . . . , Δρᵢ], derived from crystals quenched at various times, t, following initiation of the reaction by pH-shift. Here, A is scaling factor for the amplitude of the difference map density change observed at the point (x, y, z). Baseline subtraction of density at t=0 was performed prior to regression to correct for time-invariant model error.

Pre-steady-state kinetic estimates. Estimates of the pre-steady-state kinetic rate constants of BF-catalyzed primer extension were performed as previously described in (1) with minor modifications. Briefly, primer extension reaction mixtures containing 1 μM 5′-fluorescein labeled 3′-amino terminal primer, 1.25 μM DNA template, and 1-1.3 μM of BF F710Y were prepared in a buffer containing 40 mM Tris-HCl, pH 8.8, 2 mM β-mercaptoethanol (βME), and metal salts as indicated. The reaction was equilibrated at the indicated reaction temperature in a thermocycler for ˜1 min, followed by initiation by rapid addition of nucleotide substrates. Reaction samples were taken manually at various times and rapidly quenched by 1:25 dilution into 98% formamide, 10 mM tetrasodium-EDTA. Reaction samples were denatured at 95° C. for 30 sec and cooled to room temperature prior to loading and separation on 15% TBE-urea polyacrylamide gels. Fluorescent bands were visualized on a laser scanning imager (Amersham Typhoon) and analyzed using the manufacturer's software. For measurement of the elemental thio effect, reactions were carried out as above but at a reduced temperature of 45° C. due to challenges in manual sampling in the burst regime. For solvent deuterium kinetic isotope effect (SDKIE) measurements, reactions were carried out essentially as above with the following changes. First, stock reaction mixtures containing all indicated reaction components except enzyme and βME were lyophilized to dryness and reconstituted by addition of either water or 99.9% D₂O, followed by 10 mM βME from a 1 M stock solution prepared in either water or D₂O, and enzyme. For the D₂O reaction stock, the buffer was prepared at pH 8.62 prior to lyophilization, such that the final pD following resuspension was 8.8. Substrate stock solutions of 10 mM dCTP were prepared by resuspending extensively lyophilized substrate in either water or D₂O, and these stocks were in turn mixed at various ratios to furnish a series of substrate stocks with final D₂O contents of either 25, 50, 75, or 96%. Natural abundance or deuterated solvent reaction mixtures were similarly mixed at various ratios to yield a series of 9 μL reactions with final D₂O contents of 25, 50, 75, or 96% and equilibrated at 55° C. Each reaction was initiated by addition of 1 μL of substrate at the same D₂O content, and the reactions were otherwise assayed as above. The pre-steady-state rate constant, kₚₒₗ, at saturating substrate concentration, as indicated, was estimated as the linear slope of the log-transformed fraction of remaining primer, −ln(P/P₀) where P₀ is the initial primer amount, vs. reaction time at early times. Stocks of trivalent metal salts were prepared from the metal trichloride hydrate in solution with ammonium citrate (1:1 stoichiometry on a metal:citrate basis) and pH-adjusted by addition of ammonium hydroxide. This stock was added to a final concentration of 5 mM on a metal-ion basis in enzyme reactions, except where indicated.

Long NP-DNA synthesis reactions. NP-DNA strands were generated by primer extension of a 5′-fluorescein labeled 3′-amino terminal primer on a synthetic DNA template. Reactions were carried out at 55° C. with 0.9-1 μM primer, 1.2 μM template, and 250 μM of each nNTP in an NP synthesis buffer containing 40 mM Tris-HCl, pH 8.8, 10 mM βME, 25 μM spermine-HCl, and 5 mM of 1:1 ammonium citrate:ScCl₃, prepared as above. Long extension reactions typically require excess BF protein, generally ˜3 μM in the reaction. Samples of the reaction were taken at the indicated times and quenched by 1:50 dilution into 98% formamide, 10 mM tetrasodium-EDTA, denatured as above, and separated on TBE-urea gels.

Isolation of single-stranded extension products. Single-stranded primer extension products were prepared from primer extension reactions, performed as above, with the following modifications: a 3′-biotinylated DNA template oligonucleotide (+5 or +11 template sequence) was used, oligonucleotide concentrations were 2 μM primer and 2.5 μM template, and the reaction was incubated for 10 min at 55° C. in a total reaction volume of 20 μL. The extended primer strand was isolated using streptavidin-coated magnetic beads, followed by strand separation (see FIG. 19C). Briefly, 40 μL of streptavidin-coated magnetic beads from a commercial suspension at 10 mg/mL (Dynabeads MyOne Streptavidin C1) were washed briefly in 50 mM NaOH, 100 mM NaCl and re-separated using a magnetic rack, followed by three washes in the following washing buffer: 10 mM Tris pH 7.6, 100 mM NaCl, 5 mM tetrasodium EDTA, 0.1% (v/v) Tween 20 prepared in RNase-free water and filtered using a 0.2 micron sterile filter. The extension reaction was cooled to room temperature and quenched by addition of the washed magnetic bead suspension in wash buffer at a ˜2:1 ratio by volume. Binding proceeded with slow rotation at room temperature for ˜10 min-1 hr, followed by at least three washes in the same buffer. The extended strand was eluted from the beads by pH-shift, as follows. After removing the supernatant from the final wash, 22.5 μL of 0.1 N NaOH was added, and the beads were gently resuspended and then immediately separated on a magnetic rack. The supernatant was transferred to a tube containing 5 μL of 1 M Tris-HCl pH 7.0. Elution from the beads was repeated with a second fraction of 22.5 μL of 0.1 N NaOH, and the two supernatants were then pooled for a total volume of 50 μL. The eluted oligonucleotide sample was then desalted using an Oligo Clean & Concentrator Kit (Zymo Research) using the manufacturer's protocol, with elution in 20 μL LCMS-grade water.

Mass spectrometry. Single-stranded reaction products were analyzed by high resolution liquid chromatography mass spectrometry (LC-MS), essentially as described in (38). Briefly, samples were injected onto an Agilent 1200 HPLC system with online diode array detector (DAD) coupled to an Agilent 6230 time of flight (TOF) spectrometer (Agilent Technologies) in negative ion mode equipped with an electrospray ionization (Dual ESI) source with simultaneous coinjection of mass reference ions. Sample separation was performed using a 100 mm (length)×1 mm (i.d.) XBridge C18 column with a particle size of 3.5 μm (Waters) at 50° C. in an ion pairing reverse phase chromatography system. The aqueous mobile phase (solvent A) was buffered with 200 mM hexafluoroisopropanol (HFIP) with 1.25 mM triethylamine (TEA) at pH 7.0 prepared in LCMS-grade water, and the organic mobile phase was LCMS-grade methanol (solvent B). Following mass axis calibration using the manufacturer's reference ion mix, desalted samples were loaded using an autosampler in 2.5% solvent B isocratically for 4 min at a flow rate of 0.1 mL/min, with the first 2 min diverted from the MS instrument. Elution into the source was then carried out with the following gradient: 2.5% to 15% B over 16 min, 15% to 40% B over 21 min, followed by washing at 90% B. Scans were collected from 239 to 3200 m/z at 1 spectrum/s. The mass analyzer settings were as follows: drying gas flow, 8 L/min; drying gas temperature, 325° C.; nebulizer pressure, 30 psig; capillary voltage, 3500 V; fragmentor, 200 V; and skimmer, 65 V. Data analysis was performed using the manufacturer's analysis software.

Nuclease resistance assay. Approximately 10 pmol of single-stranded oligonucleotide, prepared as above, was mixed with 1 μL of 10X Exonuclease I buffer (NEB) for a total volume of 9 μL using RNase-free water and equilibrated at 37° C. Recombinant exonuclease I enzyme from E. coli (NEB) was diluted from the commercial stock in 1X Exonuclease I buffer to a concentration of ˜1 U/μL, and the reaction was initiated by addition of 1 μL (1 U) of diluted enzyme. Samples were taken at the indicated times and quenched 1:10 in 98% formamide, 10 mM tetrasodium EDTA. Control oligonucleotides extended with DNA were prepared essentially as above, except using a 3′-OH DNA primer, 0.25 mM dNTPs, and 1X Thermopol buffer (NEB) containing 2 mM MgSO4. Reaction samples were then separated and visualized by TBE-urea PAGE.

### Example 1: Reactant and Product Polymerase “Open” Complexes Containing a 3′-Amino Primer or a N3′→P5′ Phosphoramidate Bond

It was recently reported that reverse transcriptases can recognize NP-DNA templates and synthesize a complementary DNA strand, suggesting that the structural homology between NP-DNA and native nucleic acids is sufficient to support polymerase activity (Lelyveld et al., DNA polymerase activity on synthetic N3′→P5′ phosphoramidate DNA templates. Nucleic Acids Res. 47, 8941-8949 (2019)). However, enzymatic synthesis of NP-DNA would require not only recognition of the genetic polymer, but also a novel chemistry at the active site. For such catalysis to occur, the 3′-amino terminal primer would, at a minimum, need to adopt a conformation related to that seen in the native reaction center.

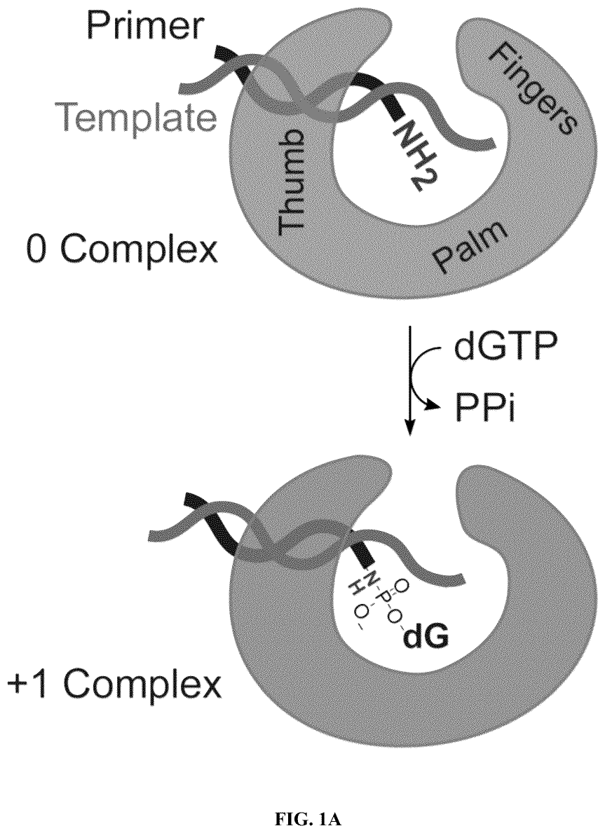

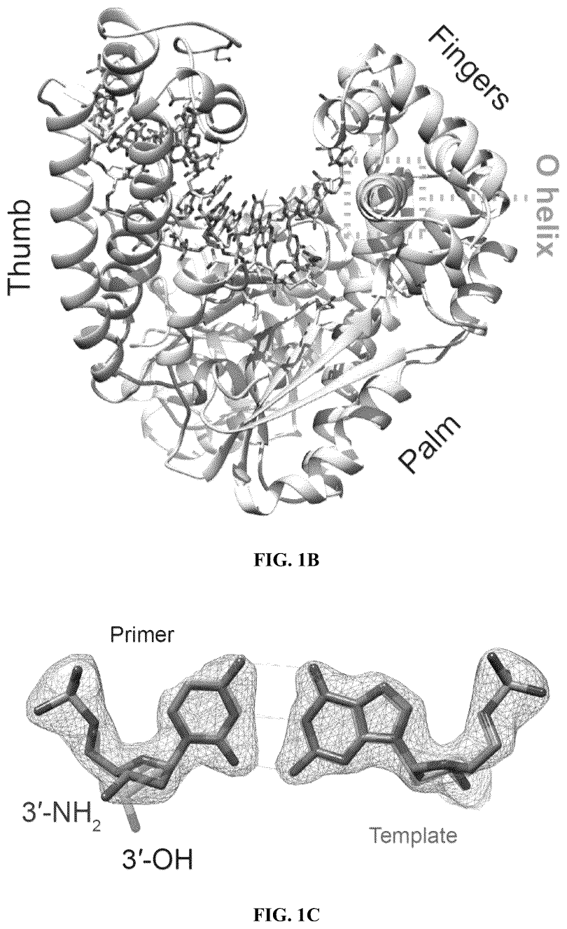

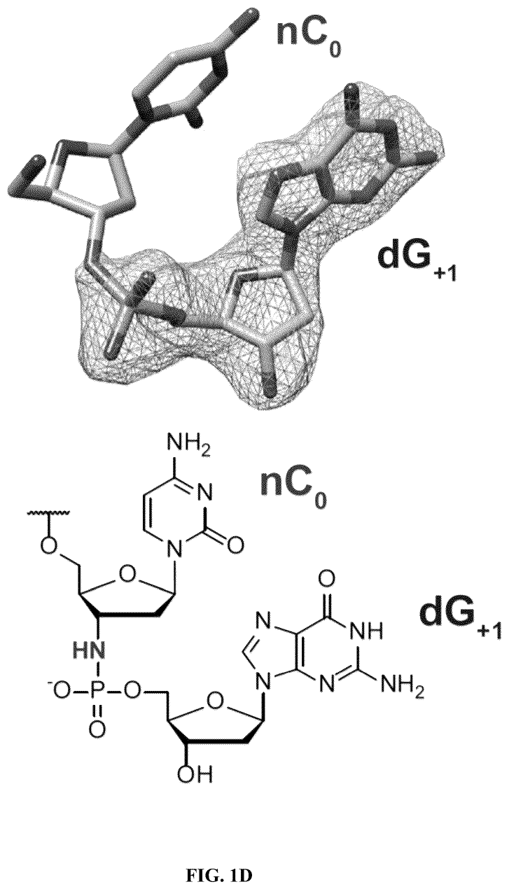

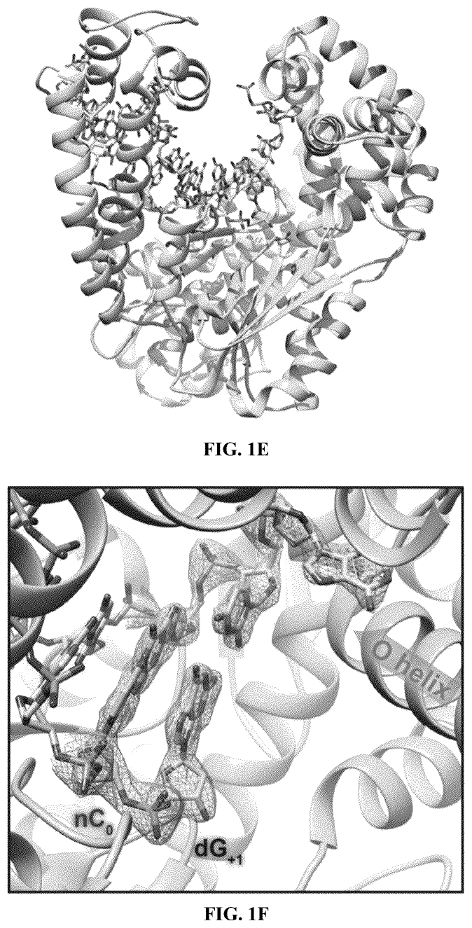

Studies described herein investigate the structural consequences of 3′-amino sugar substitution at the terminus of a primer in an enzymatic context. Crystals of the large fragment of DNA polymerase I (BF) from the thermophilic bacterium Bacillus stearothermophilus (Bst, now classified as Geobacillus) complexed with a DNA duplex in which the primer contains a terminal 3′-amino-2′,3′-dideoxycytidine (nC) residue were grown (FIGS. 1A-1F). The crystal structure of the complex was solved by molecular replacement to a resolution of 2.25 Å (FIGS. 1A-1F and Tables 2-3).

TABLE 2
Crystal X-ray data collection statistics.

| Structure | 1 | | 2 | | 3 | | 4 | |
|---|---|---|---|---|---|---|---|---|
| Space group | P2₁2₁2₁ | | P2₁2₁2₁ | | P2₁2₁2₁ | | P2₁2₁2₁ | |
| Unit cell parameters | 85.20, 93.68, 107.14, | | 87.61, 93.92, 106.12, | | 93.63, 108.07, 149.75, | | 93.76, 108.80, 149.97, | |
| (Å, °) | 90.00, 90.00, 90.00 | | 90.00, 90.00, 90.00 | | 90.00, 90.00, 90.00 | | 90.00, 90.00, 90.00 | |
| Resolution range, Å | 50-2.27 (2.36- | | 50-2.25 (2.33- | | 50-2.1 (2.18- | | 50-2.25 (2.33- | |
| (last shell) | 2.27) | | 2.25) | | 2.1) | | 2.25) | |
| Unique reflections | 39951 | | 40025 | | 89397 | | 72498 | |
| Completeness, % | 99.9 | (100) | 94.4 | (83.2) | 100 | (100) | 98.4 | (90.4) |
| R_{merge}, % | 12 | (50.8) | 14.1 | (57.4) | 8.2 | (46.2) | 12.5 | (60.5) |
| <I/σ(I)> | 17.9 | (4.7) | 15.1 | (6.8) | 23.4 | (3.55) | 10.0 | (2.67) |
| Redundancy | 8.0 | (8.0) | 7.0 | (6.0) | 7.3 | (7.3) | 3.6 | (3.3) |

TABLE 3
Crystal structure refinement statistics.

| Structure | 1 | 2 | 3 | 4 |
|---|---|---|---|---|
| PDB code | 6UR2 | 6UR4 | 6UR9 | 6US5 |
| Complexes per | 1 | 1 | 2 | 2 |
| asymmetric unit | | | | |
| Resolution range, Å | 40.85-2.27 | 28.4-2.25 | 46.8-2.1 | 47.1-2.25 |
| R_{work}, % | 22.9 | 20.5 | 20.4 | 22.4 |
| R_{free}, % | 27.7 | 24.4 | 24.3 | 27.8 |
| Number of reflections | 37883 | 40004 | 89393 | 72436 |
| Bond length R.M.S. (Å) | 0.008 | 0.013 | 0.031 | 0.055 |
| Bond angle R.M.S. | 1.55 | 1.818 | 6.642 | 7.639 |
| Average B-factors, (Å²) | 42.6 | 30.1 | 42.4 | 50.8 |

The enzyme was found in a 1:1 stoichiometry with the bound duplex in the asymmetric unit, with the nC terminated primer aligned at the canonical phosphodiester bond-forming active site (FIG. 1A, 0 Complex). Although the crystal was grown in the presence of free 2′-deoxyguanosine 5′-triphosphate (dGTP) and Mg²⁺, no ordered metal ion or nucleotide was apparent. The structure is consistent with an “open” conformation (Kiefer et al., Visualizing DNA replication in a catalytically active Bacillus DNA polymerase crystal. Nature 391, 304-307 (1998)), in which the “O-helix” of the fingers domain (698-714) is arrayed distally from the primer terminus, allowing significant solvent access to the reaction center (FIG. 1B). Density for the 3′-amino-2′,3′-dideoxyribose sugar of the terminal nC residue was consistent with a C3′-endo conformation, in concordance with the A-form geometry seen in crystallographic and NMR studies of NP-DNA duplexes (Ding et al., An Oligodeoxyribonucleotide N3′→P5′ Phosphoramidate Duplex Forms an A-type Helix in Solution. Nucleic Acids Res. 24, 354-360 (1996); Ding et al., NMR Solution Structure of the N3′→P5′ Phosphoramidate Duplex d(CGCGAATTCGCG)₂ (SEQ ID NO:7) by the Iterative Relaxation Matrix Approach. Biochemistry 37, 12082-12093 (1998); and Tereshko et al., Consequences of Replacing the DNA 3′-Oxygen by an Amino Group: High-Resolution Crystal Structure of a Fully Modified N3′→P5′ Phosphoramidate DNA Dodecamer Duplex. J. Am. Chem. Soc. 120, 269-283 (1998)) (FIG. 1C).

Under different conditions, a slow-growing crystal emerged after ˜45 days, yielding a 2.27-Å structure of an alternative view of the complex. While the solved structure was again consistent with an open conformation, it was found during refinement that the observed density for the bound duplex was best explained as a post-translocation translocated product complex containing a covalently incorporated deoxyguanosine (dG) residue at the +1 position of the primer strand (+1 complex, FIGS. 1D-1F). The density of the added dG is well-defined in the complex, as is the density corresponding to the +2 template deoxyadenosine (dA), now positioned in the proximal templating position in a pocket formed by the O and P helices of the fingers domain (FIG. 1F).

A primer extension reaction occurring on a timescale of weeks could reflect a background uncatalyzed rate or a process that occurs solely in crystallo. It was therefore sought to establish whether NP bond formation occurs in solution and, if so, whether this reaction is in fact catalyzed by the polymerase. When incubated under conditions similar to the mother liquor in which extension was first observed crystallographically, slow extension of the 3′-amino primer could be observed in a polymerase-dependent manner on a timescale of days in solution (FIG. 6). By high resolution mass analysis of the reactions, ions consistent with the NP bonded +1 product were detected with 3.4 ppm mass error and corresponding isotopic distribution, while no products of 3′-5′ exonucleolytic activity and/or extension were detected (FIG. 7A-7B).

Taken together, these results demonstrate that DNA polymerase adopts an “open” conformation to produce a N3′→P5′ phosphoramidate bond.

### Example 2: Polymerase-Catalyzed 3′-Amino Primer Extension and NP-DNA Synthesis

This example reports identification of conditions in which BF is capable of extension beyond a 3′-amino terminus in solution (FIGS. 2A-2D). Divalent metal cofactors were screened under pre-steady-state conditions and strong cofactor-dependent effects on activity were observed. Kinetics of 3′-amino primer extension with 5 mM Ca²⁺, Mg²⁺, Ba²⁺, and Sr²⁺ were all faster than in the absence of added divalent metals (FIG. 2A). The cofactor-dependent kinetics did not follow a trend consistent with the Irving-Williams series nor that expected from the aquo ion pKa. Interestingly, Ca²⁺ had the largest kinetic effect at pH 8.8 and 55° C. When titrated into the reaction, observed rate constants showed saturation behavior at similar apparent dissociation constants, K_{d,app}, for Mg²⁺ and Ca²⁺ (1.3 mM vs. 2.1 mM, respectively, in the presence of 1 mM dCTP). However, the maximum reaction velocity was ˜5-fold faster in the presence of saturating Ca²⁺ vs. Mg²⁺ (FIGS. 2B-2C), maximally ˜4.5 nt/hr with Ca²⁺. Phosphodiester-forming activity with Ca²⁺ has been reported (Irimia et al., Calcium Is a Cofactor of Polymerization but Inhibits Pyrophosphorolysis by the Sulfolobus solfataricus DNA Polymerase Dpo4. Biochemistry 45, 5949-5956 (2006); and Ralec et al., Calcium-driven DNA synthesis by a high-fidelity DNA polymerase. Nucleic Acids Res. 45, 12425-12440 (2017)). Here, two additions of dCTP were observed on a GG-containing template in the presence of either Ca²⁺ or Mg²⁺, where the first is linked via a phosphoramidate ester and the second via a phosphodiester (FIG. 2C).

With the enhanced kinetics afforded by Ca²⁺, BF was capable of catalyzing multiple-turnover condensation of nNTP monomers to form NP-DNA oligonucleotides in a DNA template-directed manner (FIG. 2D). Extension up to the +8 nt product was detected after 24 h at 55° C. in the presence of Ca²⁺ and all four nNTP substrates, but no significant extension was observed on an NP-DNA template.

Taken together, these results demonstrate that DNA polymerase forms N3′→P5′ phosphoramidate linked oligonucleotides in a template-directed manner.

### Example 3: Substrate-Dependent Kinetic Parameters for NP Activity in DNA Polymerase

Although wild-type BF has bona fide DNA-dependent NP-DNA polymerase activity, a substantial kinetic defect is associated with nNTP vs. dNTP addition to a 3′-amino terminal primer (FIG. 3A). For the first addition to a primer ending in nG, the rate constant, kₚₒₗ, in reactions with nCTP was ˜22-fold slower than with dCTP (FIG. 3B and Table 4). In seeking to rationalize this apparent recognition of the 3′-substituent, it was first considered that 3′-amino-2′,3′-dideoxyribonucleosides adopt a C3′-endo sugar conformation and, as such, bear greater conformational similarity to ribonucleosides than deoxyribonucleosides. It is therefore conceivable that nNTPs might be disfavored based on discrimination of their RNA-like sugar pucker. Selectivity against ribonucleotides purportedly involves recognition by “steric gate” residues, and mutations at these positions alter selectivity against ribonucleotide incorporation (C. M. Joyce, Choosing the right sugar: How polymerases select a nucleotide substrate. Proc. Natl. Acad. Sci. 94, 1619-1622 (1997); and Brown and Suo, Unlocking the Sugar “Steric Gate” of DNA Polymerases. Biochemistry 50, 1135-1142 (2011)). However, nNTPs and dNTPs are both chemically 2′-deoxy, despite their distinct sugar pseudorotation. Limited screening of mutations at “steric-gate” hotspot positions (FIG. 3C), as well as two highly conserved residues involved in metal cofactor binding, mainly exacerbated selectivity against nCTP (FIG. 3D). However, mutation at one position in the O helix, Phe-710, known to affect substrate preference for 2′,3′-dideoxyribonucleotide 5′-triphosphates (ddNTPs) (Tabor and Richardson, A single residue in DNA polymerases of the Escherichia coli DNA polymerase I family is critical for distinguishing between deoxy-and dideoxyribonucleotides. Proc. Natl. Acad. Sci. 92, 6339-6343 (1995); Astatke et al., How E. coli DNA polymerase I (klenow fragment) distinguishes between deoxy-and dideoxynucleotides. J. Mol. Biol. 278, 147-165 (1998); and Warren et al., The structural basis for the mutagenicity of O(6)-methyl-guanine lesions. Proc. Natl. Acad. Sci. 103, 19701-19706 (2006)), showed a significant kinetic enhancement, as well as a large effect on dCTP vs. nCTP selectivity. The mutant F710Y demonstrated a 21-fold increase in kₚₒₗ for nCTP, as well as a 2.6-fold increase for dCTP (FIG. 3E and Table 4). This mutant could achieve extension to a full length +28 nt NP-DNA product in <24 hours at 55° C. in the presence of Ca²⁺ and all four nNTPs (FIG. 3F).

TABLE 4
Substrate-dependent kinetic parameters for NP activity
in BF under pre-steady-state conditions, as in FIG. 3.

| | |
|---|---|
| kₚₒₗ | K_{d, app} |

| | | | | |
|---|---|---|---|---|
| dCTP | nCTP | dCTP | nCTP | Selectivity^{a} |

| | | |
|---|---|---|
| min⁻¹ | μM | dCTP/nCTP |

| | | | | | |
|---|---|---|---|---|---|
| WT | 0.075 | 0.0033 | 1.7 | 3.8 | 51 |
| F710Y | 0.191 | 0.069 | 1.1 | 3.6 | 9 |

^{a}Selectivity defined as the ratio (kₚₒₗ/K_{d, app})_{dCTP}/(kₚₒₗ/K_{d, app})_{nCTP}.

To establish that NP-DNA polymerase activity is in fact template-directed by BF, sequence specific stalling of primer extension in the presence or absence of individual nucleotides was analyzed. By using drop-out mixes, in which one of the four nNTPs is absent in each reaction, a substantial kinetic block at (or proximal to) the position corresponding to the absent substrate was observed (FIG. 3G, left). With drop-in mixes, in which one nucleotide of a dNTP mix is substituted with its corresponding 3′-amino analog in each reaction, BF generates a Sanger-type ladder, with accumulation of products at positions corresponding to the nNTP present in each reaction (FIG. 3G, right). Finally, the products of these long extension reactions show the acid lability characteristic of NP bonds (FIG. 3H). Incubation of the quenched reaction with acetic acid (HOAc) at 75° C. generated a cleavage ladder for all positions when nNTPs were used for 3′-amino primer extension, whereas no hydrolysis intermediates are observed when dNTPs have been used to extend the same primer.

Relative to its native phosphodiester bond forming activity, the kinetic disadvantage of BF's NP-DNA polymerase activity is approximately four orders-of-magnitude with F710Y, since transient kₚₒₗ/K_{d} is 3.1 μM⁻¹ s⁻¹ vs. 3.2×10^{-4 μ}M⁻¹ s⁻¹ for DNA synthesis with Mg²⁺ (Wang et al., Structural factors that determine selectivity of a high fidelity DNA polymerase for deoxy-, dideoxy-, and ribonucleotides. J. Biol. Chem. 287, 28215-28226 (2012)) vs. 3.2×10^{-4 μ}M⁻¹ s⁻¹ for NP-DNA synthesis with Ca2+(Table 4). This difference is of a similar order to that seen for mismatch incorporation. Unlike mismatches, the binding affinity for cognate nNTPs remains high, and the difference in catalytic efficiency is likely to arise mainly in the chemical step of the reaction. It may nevertheless be instructive to establish whether there are unique structural determinants associated with formation of the phosphoramidate catalytic complex.

### Example 4: Structural Snapshots of the Reaction Pathway in NP Catalysis

To observe the major steps in the reaction pathway for NP-DNA synthesis, from the reactant complex (FIG. 1B and FIG. 4A) to the +1 product complex (FIGS. 1E-1F and FIG. 4D), BF was co-crystallized with an unreactive nucleotide analog (dGpNHpp), a 3′-amino terminated DNA primer, and a DNA template. Using the double mutant F710Y/D598A (Warren et al., The structural basis for the mutagenicity of O(6)-methyl-guanine lesions. Proc. Natl. Acad. Sci. 103, 19701-19706 (2006), Johnson et al., Processive DNA synthesis observed in a polymerase crystal suggests a mechanism for the prevention of frameshift mutations. Proc. Natl. Acad. Sci. 100, 3895-3900 (2003)), the analog-bound complex with Mn²⁺ was crystallized and the structure was solved to 2.25 Å resolution (FIG. 4B).

Two complexes were found in the asymmetric unit, both substrate bound in a closed conformation of the fingers domain, as seen by a large conformational change in the O helix proximal to the reaction center. A single metal ion was coordinated by the α,β-imido-triphosphate moiety in each active site, although the bound metal ion and substrate is more ordered in one of the two complexes in the asymmetric unit. In this complex, the terminal 3′-amino group of the primer is in close proximity with the alpha-phosphorus of the analog (N3′-P_{α} distance ˜3.8 Å), as well as with the carboxylate group of Asp-830 in the active site (N3′-O distance ˜2.3 Å, FIG. 4C), significantly closer than the ˜2.9-3.0 Å seen in the open conformation (FIG. 4A). This highly conserved aspartate coordinates divalent metal ions in structures of the equivalent native complex when the primer bears a 3′-OH, but there is substantial debate over its potential role as a general base in native phosphodiester catalysis (Castro et al., Two proton transfers in the transition state for nucleotidyl transfer catalyzed by RNA- and DNA-dependent RNA and DNA polymerases. Proc. Natl. Acad. Sci. 104, 4267-4272 (2007); Steitz et al., A unified polymerase mechanism for nonhomologous DNA and RNA polymerases—Comment/reply. Sci. Wash. 266, 2022 (1994); and Pelletier et al., Structures of ternary complexes of rat DNA polymerase beta, a DNA template-primer, and ddCTP. Science 264, 1891-1903 (1994)). In the closed substrate-bound structure, the aspartate conformation is not significantly altered as a result of 3′-amino substitution at the primer terminus relative to that seen previously with 2′,3′-dideoxy termini (Johnson et al., Processive DNA synthesis observed in a polymerase crystal suggests a mechanism for the prevention of frameshift mutations. Proc. Natl. Acad. Sci. 100, 3895-3900 (2003)). Nevertheless, this carboxylate appears to be critical, as the mutation D830N entirely abolishes NP-DNA synthesis (FIG. 3D). On the other hand, the mutation E831Q did not substantially affect activity, although this adjacent residue is typically found coordinating a metal ion in structures modeling the native DNA polymerase activity. In the analogous nonenzymatic chemistry with phosphorimidazolides, rapid kinetics are observed in the absence of divalent metal ions, suggesting that inner sphere metal coordination is superfluous for the reactivity of the 3′-amino nucleophile (Zhang et al., Synthesis of N3′-P5′-linked Phosphoramidate DNA by Nonenzymatic Template-Directed Primer Extension. J. Am. Chem. Soc. 135, 924-932 (2013); Zhang et al., Fast and accurate nonenzymatic copying of an RNA-like synthetic genetic polymer. Proc. Natl. Acad. Sci. 110, 17732-17737 (2013); and Röthlingshöfer and Richert, Chemical Primer Extension at Submillimolar Concentration of Deoxynucleotides. J. Org. Chem. 75, 3945-3952 (2010)).

An alternative model of the catalytic complex was produced by co-crystallization with nGTP and a template-bound primer carrying a 3′-terminal 2′,3′-dideoxycytidine (ddC) residue. This complex with the BF double-mutant was crystallized and solved to 2.10 Å resolution (FIG. 4C). The resulting structure shows a single ordered Ca²⁺ and substrate in the closed conformation, but only in one of two complexes in the asymmetric unit (FIG. 5A). In the closed complex, a possible role for the mutation F710Y is suggested by the orientation of the tyrosine hydroxyl, which is in close proximity to both the β-phosphate non-bridging oxygen of the incoming substrate and its 3′-amino group. This position is similar to that seen in the presence of a dideoxynucleotide substrate (Warren et al., The structural basis for the mutagenicity of O(6)-methyl-guanine lesions. Proc. Natl. Acad. Sci. 103, 19701-19706 (2006)). The noncovalent bonding network also appears to involve the heterocyclic nitrogen of His-682 (FIGS. 4B-4C). Moderate donor-acceptor distances across this network, 2.6-2.8 Å, suggest a distributed hydrogen-bonding stabilization of the substrate and leaving group (FIG. 5A). In the dGpNHpp analog-bound reaction complex, the C2′-endo sugar conformation of the substrate analog is such that a solvent water is bound within this network (FIG. 4B), whereas the 3′-amino group of nGTP in the C3′-endo conformation appears to substitute for this water in the alternative closed complex (FIG. 4C). However, the apparent binding affinity for nCTP vs. dCTP was unaffected by F710Y with Ca²⁺, indicating that this tuning of the substrate is likely relevant only to its conformation in the closed catalytic complex.

### Example 5: Trivalent Rare Earth Metal Cofactors Confer Rapid NP-DNA Polymerase Activity

A DNA polymerase with a single mutation and divalent calcium cofactor catalyzes the synthesis of unnatural N3′→P5′ phosphoramidate (NP) bonds to form NP-DNA. However, this template-directed phosphoryl transfer activity remains orders-of-magnitude slower than native phosphodiester synthesis. We used time-resolved X-ray crystallography to show that NP-DNA synthesis proceeds with a single detectable calcium ion in the active site. Using insights from isotopic and elemental effects, we propose that one-metal-ion electrophilic substrate activation is inferior to the native two-metal-ion mechanism. We find that this deficiency in divalent activation could be ameliorated by trivalent rare earth and post-transition metal cations, dramatically enhancing NP-DNA synthesis. Scandium(III), in particular, confers highly specific NP activity with kinetics enhanced by >100-fold over calcium(II), yielding NP-DNA strands ≥100 nts in length.

The principal chemistry at the core of RNA and DNA metabolism is phosphodiester synthesis. Polymerases generate nascent strands of genetic material by stepwise phosphoryl transfer of nucleotides to primer termini, yielding O3′→P5′ phosphodiester linkages. This template-directed process is conserved across all known biology. Nucleotide 3′ substitutions have therefore been widely regarded as chain terminating for polymerase activity, forming the basis for a class of nucleoside analog drugs (1, 2).

We have demonstrated the direct enzymatic synthesis of an unnatural linkage by substitution of the 3′-OH nucleophile with a 3′-amine, extending the chemistry amenable to polymerase catalysis (1). We reported that 3′-NH2 primer extension can be catalyzed by a modified DNA polymerase, yielding N3′→P5′ phosphoramidate DNA (NP-DNA, FIGS. 8A-8B). The large fragment of DNA polymerase I (BF), cloned from the thermophilic soil bacterium Geobacillus stearothermophilus (Bst), acquires nontrivial levels of N3′→P5′ polymerase activity via two surprisingly minor substitutions: (A) a single active site mutation (F710Y) and (B) substitution of its divalent Mg2+ cofactors with Ca²⁺ (1). However, this level of activity remained around four orders-of-magnitude slower than native phosphodiester synthesis. Although phosphoramidate (NP) synthesis was detectable in the presence of several divalent alkaline earth metal ions, the pattern of metal cofactor activity was distinct from that found in native phosphodiester activity (3). Crystal structures of the ground-state NP reaction complex showed a single divalent metal ion in the active site (1), suggesting a plausible distinction between the NP mechanism and the classical two-metal-ion mechanism in native phosphodiester activity (4). Whether NP catalysis does in fact rely on a distinct mechanism with a nonclassical cofactor configuration is established herein, as do the critical factors limiting the kinetics of NP synthesis.

### Example 6: Linearly Correlated Density Dynamics in Reacting BF Crystals During NP Synthesis

To understand the mechanistic barriers to rapid enzymatic NP-DNA synthesis, we first sought to observe NP bond formation through crystallography. In previous x-ray crystal structures of the fully assembled NP polymerase pre-insertion complex, we modeled a single divalent metal ion in the reaction center, occupying a site distal to the primer-terminal nucleophile (1). This site is equivalent to the so-called “B site” in the classical two-metal-ion reaction center and includes inner sphere ligands from the substrate triphosphate moiety, the side chains of aspartates 830 and 653, and the backbone amide of Tyr-654. No evidence for an “A-site” metal proximal to the primer 3′-amino terminus was observed in these earlier pre-chemistry model structures, a finding consistent with the expectation that a neutral amine is the nucleophile in NP synthesis. However, earlier structures did not fully recapitulate the active NP reaction center or the product state (1). The A-site metal ion can also be poorly ordered even in structures of the native polymerase reaction complex. Nakamura et al. reported that accumulation of an additional metal ion in a “C site” of polymerase η, a polymerase Y family member, occurs in a manner linearly correlated with bond formation, suggesting that the product state's post-chemistry metal configuration may be distinct from that observed in the pre-chemistry reaction complex (5). This observation was subsequently extended to polymerase X family members (6, 7). To add evidence that a single divalent metal ion cofactor catalyzes unnatural NP synthesis in BF, a polymerase A family member, we carried out a single nucleotide primer extension reaction in intact crystals (FIGS. 8B-8D). We crystallized active substrate-bound BF polymerase F710Y/D598A with a 3′-amino terminal DNA primer and DNA template in the presence of Ca²⁺ and 2′-deoxyguanosine 5′-triphosphate (dGTP). Reactions were then initiated in the intact crystals by pH-shift from an acidic mother liquor (pH<6) to a basic soaking liquor at pH 8.8, and reacting crystals were quenched at various times by flash freezing (FIGS. 8C-8D).

Quantitative analysis of electron density dynamics is complicated by crystal-to-crystal variance in diffraction datasets. We therefore incorporated datasets arising from up to 4 crystals at each of 6 time points following initiation. By scaling reflections from 19 total crystals (resolution 2.0-2.7 Å) across the time course (0-24 h), we could estimate voxel-wise first-order rate constants for changes in real-space electron density across the asymmetric unit and particularly at the reaction center (FIG. 8D, bottom left). Early time points show negligible differences in ordered density between the ground state complex crystallized under acidic conditions versus the complex after 1 h of soaking under reaction conditions, suggesting that the reactant state and ground state cannot be clearly distinguished in this resolution range. By following the reaction at subsequent times, we observed monotonic accumulation of nascent NP bond density between the primer terminal 3′-amino group and the substrate α-phosphate with concomitant loss of electron density between the α- and β-phosphates with similar first-order rate constants (FIG. 8D).

By inspecting the kinetics of density changes at nearby sites, we noticed that the most prominent local density dynamics in the active site had simple linear relationships with nascent bond formation. Pairwise linear regression yields the relative slope, β, of density changes occurring at any two points across the time-resolved dataset (FIGS. 9A-9B). Performing this regression at all points in the asymmetric unit vs. the nascent bond furnished a field of β values or “beta map” in real space, which could be contoured at a desired threshold of |β| to give isosurfaces for visualization (FIG. 9C). To show that regions with high |β| are also highly correlated, we produced pairwise Pearson correlation maps by a similar procedure (FIG. 2D). Regions of local conformational dynamics were qualitatively in agreement between correlation maps with coefficient, r, contoured at |r|>0.9 (FIG. 9D) vs. regression maps contoured at |β|>0.45 (FIG. 9C). When beta maps were contoured at |β|>0.9, we observed that the included voxels were tightly constrained to the site of the nascent NP bond and the scissile P_{α}−O_{α,β} bond (FIG. 13A). No other linear density dynamics are larger in scalar magnitude than the chemistry itself during bond formation in crystallo.

Inspecting the isosurfaces contoured at |β|>0.45, we observed four major density perturbations that are also highly correlated (|r|>0.9) with the nascent bond (FIG. 9B, insets i-iv): (i) displacement of the terminal phosphodiester linkage between the −1 position and the primer terminus, (ii) a conformational switch of the substrate deoxyribose moiety from C2′-endo to C3′-endo, matching the sugar pucker of the primer terminus, (iii) a small deflection of the K706 side chain toward the substrate bridging α,β-oxygen, homologous to a putative general acid in the native mechanism (8), and finally (iv) disordering of the leaving group pyrophosphate moiety. As apparent from the high correlation coefficients, maps generated as 1−p for two-tailed p-values determined for the correlation field vs. the nascent bond show that all of these active site dynamics are highly significant with p<10⁻⁶ (FIG. 13B). Deflection of K706 toward the substrate α,β-bridging oxygen is well-resolved on the beta map as positive and negative lobes surrounding terminal atoms of the side chain (FIG. 9E). This motion is noteworthy because the site is structurally homologous to the C-site metal-ion density reported for X-and Y-family polymerases (5-7, 9). Crucially, no well-correlated density changes were observed at a position equivalent to the absent A-site metal in the native mechanism (FIG. 9F).

Conformational changes in both the upstream and downstream nucleotides flanking the nascent bond are linearly correlated with covalent bonding (FIG. 9B insets i and ii, and FIGS. 9C-9D). The upstream conformational change at the primer-terminal linkage is also observable in time-resolved datasets of native phosphodiester synthesis in polymerase η (5). The downstream conformational change in the dGTP substrate sugar from C2′-to C3′-endo upon product formation (FIG. 9B inset ii and FIG. 9E), however, was not observed in an equivalent time-resolved experiment with the wild-type enzyme, in which the conformation is stably C3′-endo (FIGS. 14A-14E). Although intriguing, a ground state conformational change in the deoxynucleotide substrate is unlikely to be relevant to 3′-amino nucleotides, which already prefer the C3′-endo conformation due to a mixture of steric and anomeric effects (10).

Comparative analysis of density dynamics in wild-type versus F710Y BF. We further observed that the ground state substrate conformation is distinct between the mutant F710Y and wild-type BF. This raises the possibility that the role of the activating mutation F710Y could be, at least in part, to induce the observed C2′-endo substrate conformation. F710Y does modestly enhance the pre-steady-state rate constant for incorporation of deoxynucleotide substrates by ˜2.5-fold in solution with a minor improvement in the kinetically-determined apparent dissociation constant, K_{d,app} (1). However, the furanose moiety of 3′-amino nucleotides is generally understood to prefer the C3′-endo conformation due to a mixture of steric and anomeric effects. The 3′-amino sugar was indeed found as C3′-endo in the inactive reaction complex containing nGTP substrate and a dideoxy-terminal primer, as well as in the incorporated terminal 3′-amino product in a structure of the BF-bound translocated state following primer extension (1). Efforts to obtain crystals with 3′-amino groups on both the primer and substrate have so far been unsuccessful. If adoption of a C2′-endo conformation in the substrate were critical to the enhancing effect of F710Y, we would not expect such an enhancement for nNTPs. Yet the rate constant for incorporation of 3′-amino substrates was indeed enhanced by over 20-fold by the mutation, substantially more than for native deoxynucleotide substrates and with almost no effect on Kd,app. We therefore infer that the conformational shift of dGTP in reacting BF F710Y crystals is unlikely to explain the enhanced kinetics for nNTP substrates afforded by the mutation.

Because this single activating mutation has such a large effect on the overall NP-DNA synthesis kinetics, we searched for alternative mechanistic explanations using a time-resolved dataset produced with the wild-type parent enzyme. As the reaction proceeds in wild-type crystals, a new region of density consistent with a solvent molecule accumulates within the Ca²⁺ ligand sphere (Ca²⁺-H2O distance ˜3.2 Å), in a manner highly correlated with the formation of the nascent bond and concomitant with a subtle shift in the Ca²⁺ ion position (FIGS. 14A-14E). The correlated emergence of this water in the wild-type active site (FIG. 14C and FIG. 14E) is not observed in the F710Y mutant, although density at the proximal β-phosphate substrate position becomes substantially disordered in both mutant and wild-type structures over time. In the mutant, a water at the equivalent site remains stably ordered throughout the reaction. One apparent consequence of the presence of this water is to alter the preferred Asp-653 ligand interaction from a predominantly bidentate to a monodentate configuration on the distal side of the ion (39). The coordination sphere ultimately found in the wild-type product state has therefore become more similar to the F710Y ground state, suggesting that the role of the activating mutation is to stabilize a favorable product-like metal-ligand geometry.

Data collection, phasing, and refinement statistics of the determined structures are listed in Tables 5-6.

TABLE 5
Crystal X-ray data collection statistics.

| Item | F710Y 0 h (GS) | | F710Y 1 h | | F710Y 2 h | | F710Y 4 h | |
|---|---|---|---|---|---|---|---|---|
| PDB code | 8SCG | | 8SCI | | 8SCJ | | 8SCK | |
| Beamline | 8.2.2 | | 8.2.2 | | 8.2.2 | | 8.2.2 | |
| Wavelength (Å) | 1.0000 | | 1.0000 | | 1.0000 | | 1.0000 | |
| Space group | P2₁2₁2₁ | | P2₁2₁2₁ | | P2₁2₁2₁ | | P2₁2₁2₁ | |
| Unit cell parameters (Å, °) | 93.68, 108.81, | | 93.16, 108.54, | | 93.03, 109.70, | | 93.54, 108.97, | |
| | 149.55, 90, 90, 90 | | 148.78, 90, 90, 90 | | 149.84, 90, 90, 90 | | 149.40, 90, 90, 90 | |
| Resolution range (Å) | 50.00-2.00 (2.05- | | 50.00-2.68 (2.73- | | 50.00-2.67 (2.72- | | 50.00-2.30 (2.34- | |
| | 2.00) | | 2.68) | | 2.67) | | 2.30) | |
| Unique reflections | 99476 | (6410) | 43114 | (2134) | 41492 | (2033) | 66713 | (3344) |
| Completeness (%) | 96.5 | (88.4) | 99.6 | (100) | 96.8 | (97.3) | 97.6 | (100) |
| Redundancy | 9.0 | (7.8) | 7.2 | (7.3) | 4.7 | (4.8) | 4.8 | (4.9) |
| R_{merge} (%) | 10.9 | (44.9) | 10.8 | (46.3) | 8.2 | (45.3) | 9.7 | (43.2) |
| <I/σ(I)> | 16.7 | (5.7) | 16.4 | (5.5) | 14.8 | (2.6) | 12.7 | (3.8) |

| Item | F710Y 6 h | | F710Y 8 h | | F710Y 24 h (PS) | |
|---|---|---|---|---|---|---|
| PDB code | 8SCL | | 8SCM | | 8SCN | |
| Beamline | 8.2.2 | | 8.2.2 | | 8.2.2 | |
| Wavelength (Å) | 1.0000 | | 1.0000 | | 1.0000 | |
| Space group | P2₁2₁2₁ | | P2₁2₁2₁ | | P2₁2₁2₁ | |
| Unit cell parameters (Å, °) | 93.60, 109.74, | | 93.97, 109.48, | | 93.84, 108.79, | |
| | 150.31, 90, 90, 90 | | 149.82, 90, 90, 90 | | 149.90, 90, 90, 90 | |
| Resolution range (Å) | 50.00-2.44 (2.48- | | 50.00-2.30 (2.34- | | 50.00-2.30 (2.34- | |
| | 2.44) | | 2.30) | | 2.30) | |
| Unique reflections | 54903 | (2768) | 67705 | (3421) | 66403 | (3372) |
| Completeness (%) | 94.5 | (97.5) | 97.7 | (100) | 96.7 | (100) |
| Redundancy | 8.3 | (8.4) | 4.8 | (4.8) | 7.3 | (7.2) |
| R_{merge} (%) | 10.1 | (49.2) | 8.8 | (49.8) | 9.3 | (48.3) |
| <I/σ(I)> | 16.7 | (3.9) | 14.4 | (3.0) | 21.7 | (4.9) |

| Item | WT 0 h (GS) | | WT 1 h | | WT 2 h | | WT 4 h | |
|---|---|---|---|---|---|---|---|---|
| PDB code | 8SCO | | 8SCP | | 8SCQ | | 8SCR | |
| Beamline | 23-ID-B | | 23-ID-B | | 23-ID-B | | 23-ID-B | |
| Wavelength (Å) | 1.033175 | | 1.033175 | | 1.033175 | | 1.033175 | |
| Space group | P2₁2₁2₁ | | P2₁2₁2₁ | | P2₁2₁2₁ | | P2₁2₁2₁ | |
| Unit cell parameters (Å, °) | 94.05, 109.04, | | 93.86, 108.75, | | 93.66, 108.55, | | 93.77, 109.08, | |
| | 150.94, 90, 90, 90 | | 149.99, 90, 90, 90 | | 148.25, 90, 90, 90 | | 151.18, 90, 90, 90 | |
| Resolution range (Å) | 50.00-1.92 (1.95- | | 50.00-2.08 (2.12- | | 50.00-2.18 (2.22- | | 50.00-2.00 (2.03- | |
| | 1.92) | | 2.08) | | 2.18) | | 2.00) | |
| Unique reflections | 111554 | (4420) | 90185 | (4558) | 78539 | (3916) | 101401 | (5214) |
| Completeness (%) | 93.6 | (75.6) | 97.0 | (100) | 98.8 | (100) | 96.3 | (100) |
| Redundancy | 6.3 | (5.4) | 6.0 | (6.4) | 6.1 | (6.5) | 6.4 | (6.6) |
| R_{merge} (%) | 5.5 | (38.9) | 7.9 | (47.1) | 9.5 | (49.5) | 4.8 | (44.9) |
| <I/σ(I)> | 29.9 | (4.1) | 21.7 | (4.2) | 19.2 | (4.1) | 34.6 | (4.2) |

| Item | WT 8 h | | WT 24 h | | WT 48 h (PS) | |
|---|---|---|---|---|---|---|
| PDB code | 8SCS | | 8SCT | | 8SCU | |
| Beamline | 23-ID-B | | 23-ID-B | | 23-ID-B | |
| Wavelength (Å) | 1.033175 | | 1.033175 | | 1.033175 | |
| Space group | P2₁2₁2₁ | | P2₁2₁2₁ | | P2₁2₁2₁ | |
| Unit cell parameters (Å, °) | 93.62, 108.68, | | 94.32, 108.77, | | 94.08, 109.16, | |
| | 149.25, 90, 90, 90 | | 149.57, 90, 90, 90 | | 150.59, 90, 90, 90 | |
| Resolution range (Å) | 50.00-2.10 (2.14- | | 50.00-2.34 (2.38- | | 50.00-2.38 (2.42- | |
| | 2.10) | | 2.34) | | 2.38) | |
| Unique reflections | 89558 | (4432) | 63660 | (3215) | 61520 | (3062) |
| Completeness (%) | 99.7 | (100) | 97.5 | (100) | 98.7 | (99.8) |
| Redundancy | 6.2 | (5.9) | 6.1 | (6.5) | 4.7 | (4.7) |
| R_{merge} (%) | 8.7 | (50.5) | 10.7 | (48.5) | 10.7 | (49.7) |
| <I/σ(I)> | 19.4 | (3.8) | 13.9 | (2.9) | 11.8 | (2.8) |

TABLE 6
Crystal structure refinement statistics.

| Item | F710Y 0 h (GS) | F710Y 1 h | F710Y 2 h | F710Y 4 h |
|---|---|---|---|---|
| PDB code | 8SCG | 8SCI | 8SCJ | 8SCK |
| Resolution range (Å) | 45.32-2.00 | 42.81-2.67 | 37.46-2.68 | 39.63-2.30 |
| Number of reflections | 96822 | 42855 | 40379 | 66267 |
| R_{work} (%) | 20.2 | 18.4 | 20.4 | 19.4 |
| R_{free} (%) | 24.5 | 25.6 | 26.0 | 24.8 |
| Bond length R.M.S. (Å) | 0.007 | 0.008 | 0.002 | 0.008 |
| Bond angle R.M.S. (°) | 0.91 | 0.96 | 0.51 | 0.91 |
| Average B-factors (Å²) | 34.44 | 39.48 | 41.96 | 34.62 |

| Item | F710Y 6 h | F710Y 8 h | F710Y 24 h (PS) |
|---|---|---|---|
| PDB code | 8SCL | 8SCM | 8SCN |
| Resolution range (Å) | 40.98-2.44 | 44.10-2.30 | 44.11-2.30 |
| Number of reflections | 53540 | 65830 | 66193 |
| R_{work} (%) | 19.6 | 19.3 | 18.6 |
| R_{free} (%) | 26.8 | 25.00 | 24.3 |
| Bond length R.M.S. (Å) | 0.008 | 0.008 | 0.008 |
| Bond angle R.M.S. (°) | 0.96 | 0.93 | 0.90 |
| Average B-factors (Å²) | 40.60 | 32.13 | 32.12 |

| Item | WT 0 h (GS) | WT 1 h | WT 2 h | WT 4 h |
|---|---|---|---|---|
| PDB code | 8SCO | 8SCP | 8SCQ | 8SCR |
| Resolution range (Å) | 41.52-1.92 | 39.86-2.08 | 46.83-2.18 | 44.39-2.00 |
| Number of reflections | 111220 | 90010 | 78029 | 100335 |
| R_{work} (%) | 18.9 | 18.9 | 18.8 | 19.4 |
| R_{free} (%) | 23.4 | 23.8 | 23.6 | 23.6 |
| Bond length R.M.S. (Å) | 0.007 | 0.007 | 0.008 | 0.008 |
| Bond angle R.M.S. (°) | 0.90 | 0.92 | 0.91 | 0.93 |
| Average B-factors (Å²) | 26.31 | 30.37 | 34.53 | 31.65 |

| Item | WT 8 h | WT 24 h | WT 48 h (PS) |
|---|---|---|---|
| PDB code | 8SCS | 8SCT | 8SCU |
| Resolution range (Å) | 45.24-2.10 | 47.16-2.34 | 47.04-2.38 |
| Number of reflections | 89344 | 63462 | 60654 |
| R_{work} (%) | 19.0 | 18.7 | 18.7 |
| R_{free} (%) | 23.3 | 23.2 | 23.9 |
| Bond length R.M.S. (Å) | 0.007 | 0.007 | 0.008 |
| Bond angle R.M.S. (°) | 0.90 | 0.93 | 0.95 |
| Average B-factors (Å²) | 30.05 | 35.31 | 42.06 |

### Example 7: Isotopic and Elemental Substitution Effects Point Toward Rate-Limiting Chemistry with Ca²⁺

Reacting crystals are sampled on a timescale vastly slower than that of conformational dynamics like furanose pseudorotation. The crystal data is therefore not directly informative of the relative sequence and magnitude of these reaction barriers. To gain more insight into the rate-limiting step for NP synthesis, we turned to solution-phase kinetics. Two proton transfers have been detected in the rate-limiting step of native polymerase activity using careful measurements of the solvent deuterium kinetic isotope effect (SDKIE) (8, 11). In NP synthesis, it is minimally required that one proton arising from the 3′-amino nucleophile must ultimately be transferred out of the reaction center in the forward reaction, yielding the product phosphoramidate. The conserved Asp-830 side chain, proximal to the nucleophilic primer 3′-amine at ˜2.5 Å in refined ground state structures (FIG. 10B), is the most likely general base mediating this proton transfer. A sterically-conservative point mutation at this position, D830N, entirely abolishes activity (1). We found that the effect of varying mole fraction, n, of D₂O solvent on pre-steady-state NP reaction kinetics was negligible, yielding an SDKIE estimate of 1.16 for 3′-amino primer extension in the presence of Ca²⁺ and dCTP (FIG. 10A). In native phosphodiester polymerases, this value has generally been measured in the 2-5 range (8). The substantially lower value measured here suggests that proton transfer is not a critical barrier in the NP reaction with Ca²⁺.

If the chemical step of NP bond synthesis sets the overall reaction rate, then modulating the electrophilicity of the substrate should show an appreciable kinetic effect. We therefore measured the non-bridging elemental thio-substitution effect in solution using stereopure Sp or Rp diastereomers of dCTPαS. We found that the thio effect was k_{O}/k_{S}=10.6±0.5 (mean±s.d.) when substituting the dCTP substrate with Sp-dCTPαS in pre-steady-state extension of a 3′-amino terminus at 45° C. (FIGS. 10C-10D). Elemental thio effects of similar magnitude have been observed for native polymerase mismatch extension or when the divalent cofactors are Mn²⁺ rather than Mg²⁺, both conditions for which the chemical step appears to be rate-limiting (11, 12). The thio effect was compounded by an additional 4-fold for the Rp substrate vs. the Sp substrate (FIG. 10D), consistent with the coordination geometry seen in crystal structures (FIG. 10B). The interpretation of thio effects has long been a matter of debate in phosphoryl transfer catalysis, given the potential for complicating steric effects with phosphorothioate substrates (12, 13). Nevertheless, a plausible interpretation of the observed thio effects is that the chemical step sets the overall reaction rate for NP synthesis.

### Example 8: Electrophilic Substrate Activation as a Probable Deficiency in NP Catalysis with Ca²⁺

If the chemical step is in fact rate-limiting but proton transfer is not, an augmented barrier to NP vs. phosphodiester synthesis could, at least in part, originate from an altered metal-ion configuration in the presence of a 3′-amino primer. In the native reaction center, it is generally understood that the A-site metal ion activates the 3′-OH nucleophile by inner sphere coordination, yielding a metal-alkoxide in the transition state, but numerous crystallographic models of reaction intermediates also show that the octahedral ion bound at the A site forms an additional inner sphere contact with the substrate pro-Rp α-phosphate non-bridging oxygen (14). It has been independently argued that the role of the conserved polymerase dinuclear metal center (FIG. 11A) is to stabilize the transition state electrostatically on the basis of linear free energy relationships (LFERs) obtained from Brønsted plots of pre-steady-state kinetics in polymerase β, harnessing a series of O_{β,γ} bridge substituent-modified substrate analogs with varying leaving group pKₐ (15, 16). The catalytic effect of the A-site metal in native activity likely encompasses several effects, but any catalytic effect conferred by the presence of an A-site metal ion on substrate activation—as opposed to nucleophile activation—is absent for NP synthesis. The lack of electron density for an A-site metal ion in the ground state and at later time points is also consistent with the expectation of a neutral nucleophile in the NP reaction and the generally weak affinity of aliphatic amines for Ca²⁺. Since neither the apparent binding constant nor the active site conformation are significantly perturbed by the presence of a 3′-amino nucleophile (1), a significant contributor to the kinetic defect for NP vs. OP synthesis may therefore be the relative loss of transition state stabilization associated with the missing A-site metal cofactor for the reaction with an amino nucleophile.

If the native metal cofactors act, at least in part, on the native transition state by charge stabilization, it is noteworthy that the net effect of the absence of one divalent ion (with disengagement of its conserved ligand Glu-831) would be to decrease the formal net charge of the transition state complex by one (FIGS. 11A-11B). Outer sphere charge mutations may not be able to compensate for this inner sphere defect, particularly if the role of the missing metal in native activity is, in part, to contribute to stabilizing the developing charge polarization across the scissile bond. This view is consistent with the observation that eliminating the negative charge on the disengaged native ligand by the mutation E831Q fails to enhance NP synthesis kinetics (1).

### Example 9: Trivalent Metal Ions Confer Rapid and Specific NP Synthesis Activity

On the basis of this activation argument and the observed thio effect, we hypothesized that substitution of a trivalent cation into the metal site B might compensate for the electrostatic component of the catalytic defect resulting from the absence of the A-site metal, following the hypothetical reaction structure depicted in FIG. 11C. Upon screening a series of redox-stable trivalent metal ions, we indeed found that an exotic series of trivalent metal ions could act as polymerase cofactors for rapid catalysis of NP bond formation (FIGS. 12A-12D). The diamagnetic group 3 trivalent rare earth element (REE) cations scandium (Sc³⁺), yttrium (Y³⁺), and lutetium (Lu³⁺), as well as the post-transition metal ion indium (In³⁺), all significantly improved single-nucleotide 3′-amino primer extension (FIG. 12A), as well as NP-DNA polymerization with all four nNTPs on mixed sequence templates (FIG. 12B, FIGS. 15A-15B, and FIGS. 16A-16D). Sc³⁺, in particular, accelerates pre-steady-state rate constants for nCTP addition by ˜100-fold at 55° C. to 7.1±0.5 min⁻¹ (mean±s.e.m) vs. 0.069 min⁻¹ for Ca²⁺ (1), suggesting a stabilization effect of ca. −3 kcal/mol. Lu³⁺ yields similar levels of burst activity, kₚₒₗ=6.9±0.7 min⁺¹ (mean±s.e.m), but had far weaker multiple turnover activity in long primer extensions relative to Sc³⁺ (FIG. 12B and FIGS. 15A-15B). NP-active trivalent ion cofactors have a wide range of pKₐ values for the aquo ion, are prone to hydrolysis under the reaction conditions, and have a tendency to precipitate nucleotides and other phosphates. We found that reaction conditions were optimal when these ions were buffered at ˜1:1 stoichiometry with citrate, such that all reaction components remain homogeneously soluble (FIGS. 16A-16D). Although the reaction kinetics were quite sensitive to the metal:citrate stoichiometry, they were not appreciably sensitive to the concentration of 1:1 Sc³⁺:citrate in the low millimolar range (0.5-10 mM) in the presence of 1 mM total nNTPs (FIGS. 16A-16D). However, it is known that the metal-ligand stoichiometry of trivalent REE citrate complexes in solution is diverse (17). See Table 7 for oligonucleotide sequences.

Trivalent metal ion cofactors confer exquisitely specific catalysis of NP vs. phosphodiester chemistry. We found that the pre-steady-state rate constant for extension of a native DNA 3′-OH terminus with nCTP in the presence of Sc³⁺ was 5.5±0.6×10⁻³ min⁻¹, which is at least three orders of magnitude slower than 3′-NH₂ extension (FIG. 15B). NP-DNA synthesis activated by Sc³⁺ is also inhibited by added Mg²⁺ in the low millimolar range optimal for native activity (FIG. 17A). The mutation D830N is completely inactivating, whereas the adjacent E831Q mutant exhibits wild-type activity in long extensions (FIG. 17B), consistent with the active site configuration proposed in FIG. 11C. Another remarkable feature of trivalent NP catalysis is an apparent inverse elemental thio effect. We found that the Sc³⁺ thio effect was 0.58±0.07 (mean±s.d.) at 45° C., indicating a significant preference for phosphorothioate substrates to yield thiophosphoramidate (NPS) linkages (FIGS. 12C-12D). Inverse thio effects are more readily explained in the context of inverted stereospecificity with thiophilic metal cofactor substitution, but in this case the catalyzed reaction remains highly specific (66-fold) for the Sp over Rp substrates at 45° C. Synthesis of the potentially clinically-relevant NPS linkages under these conditions was confirmed by high resolution mass spectrometry (FIGS. 18A-18D).

NP-DNA synthesis on long templates was aided by the addition of polyamines (FIGS. 16A-16D). With spermine and Sc³⁺, full length products on mixed sequence DNA templates up to +71 nt or, with additional enzyme, up to +100 nt could be synthesized in 1-2 hr at 55° C. (FIG. 12B and FIGS. 16A-16D). Polyamine rescue is consistent with linkage-dependent conformational effects on duplex helicity for NP-DNA:DNA hybrids that arise during long-range primer extension synthesis, or it might alternatively reflect competition for inhibitory metal coordination sites. Activation by spermine was optimal at low micromolar concentrations, consistent with its reported effects on Klenow activity previously attributed to helicity effects (18). Under these conditions, NP-DNA synthesis remains highly determined by template sequence, as omission of any single aminonucleotide from the nNTP mix dramatically stalls extension activity at or immediately before the first template position complementary to the missing substrate (FIGS. 19A-19BB). Extension products also show characteristic features expected from the incorporation of unnatural NP linkages, such as acid lability at elevated temperature (FIGS. 19A-19B: HOAc lanes) and high resistance to 3′-5′ exonuclease I activity (FIGS. 19C-19D).

TABLE 7
Oligonucleotide sequences.

| Oligonucleotide | Sequence (5′→3′) |
|---|---|
| Primer for crystallizations | GCGATCAGnC (SEQ ID NO: 2) |
| Template for crystallizations | ACACGCTGATCGCA (SEQ ID NO: 3) |
| Primer for solution phase reactions | Fluorescein-GCGTCCATTCGTTACCTnG (SEQ ID NO: 4) |
| Template for solution phase kinetics | TCAGGCAGGTAACGAATGGACGC (SEQ ID NO: 7) |
| Control primer with terminal 3′-OH | Fluorescein-GCGTCCATTCGTTACCTG (SEQ ID NO: 4) |
| +5 template (3′ biotinylated) | TCAGGCAGGTAACGAATGGACGC-Biotin (SEQ ID NO: 7) |
| +11 template (3′ biotinylated) | CACGCATCAGGCAGGTAACGAATGGACGC-Biotin (SEQ ID |
| | NO: 8) |
| +28 template | CGGAAATGTTGAATACTCACGCATCAGGCAGGTAACGA |
| | ATGGACGC (SEQ ID NO: 9) |
| +71 template | TTAGGTGATACCCGCCGCGTTAACGTATTCGTGCAGAAC |
| | CATATGGTCACGCATGGTGAACTCCTTCAAAACAGGTAA |
| | CGAATGGACGC (SEQ ID NO: 10) |
| +100 template | CATGTTGAATACTCACGCANNNNNNNNNNTTAGGTGAT |
| | ACCCGCCGCGTTAACGTATTCGTGCAGAACCATATGGTC |
| | ACGCATGGTGAACTCCTTCAAAACAGGTAACGAATGGA |
| | CGC (SEQ ID NO: 11) |

Note:
Base codes marked with the prefix “n” are 3′-amino-2′,3′-dideoxynucleotides. All other codes correspond to standard DNA base codes.

### Discussion

Unlike for native phosphodiester catalysis, only a single metal ion cofactor is detectable by crystallography during NP catalysis. Although transition states and other short-lived intermediates are of course not directly observable in our studies, the totality of the evidence supports a single metal ion mechanism with weakened substrate activation in 3′-amino primer extension by BF. From this inference, we identified a series of trivalent metal ion polymerase cofactors with dramatically enhanced reactivity and specificity for NP-DNA synthesis. Relative to the wild-type BF DNA polymerase, combining a single active-site point mutation with trivalent REE cofactor substitution accelerates NP synthesis by well over 1000-fold, yielding template-directed NP-DNA strands of useful lengths in benchtop enzyme reactions. In the presence of Sc³⁺ and spermine, overall NP polymerase reaction time to yield full length products (˜0.9-1.3 min/nt at 55° C.) was similar to that reported for certain evolved xenonucleic acid (XNA) polymerases producing OP-linked strands incorporating synthetic sugars (0.75-1.5 min/nt at 50-65° C. (19)). REE-catalyzed NP polymerase reactions are therefore comparable to those of extensively mutated polymerases engineered for alternative phosphodiester synthesis activities by directed evolution (20). This finding suggests that the evolutionary distance to catalysis of distinct chemistry may be negligible when alternative cofactors are present, given a conservatively modified substrate. Recovering a conserved chemistry with highly divergent substrates, by comparison, may require a more extensive search of sequence space.

The considerable catalytic advantage conferred by trivalent metal ion cofactors implies that a broader family of polymerase activities may be accessible by directed evolution of substrate specificity, given the well-demonstrated ability of evolutionary methods to optimize native catalysis on XNA substrates (20, 21). NP-DNA falls within a class of phosphoramidate nucleic acids that have long been valued for their potential utility as nuclease-resistant antisense oligonucleotides (ASOs) (22). These polymers have also been extensively studied for their nonenzymatic self-assembly from chemically-activated phosphorimidazolide nucleotide analogs (23-26), on a path to developing synthetic protocells and model systems for key aspects of abiogenesis (27, 28). Despite lacking a 2′-OH group, short synthetic NP-DNA duplexes adopt a conformational geometry more similar to RNA than DNA (10), such that these polymers are viable templates for reverse transcriptase (RT) activity (29) but are poor substrates for several classes of nucleases (30, 31). Whether NP polymers are, like RNA, fully functional Darwinian polymers remains a matter of significant interest. The development of practical levels of NP polymerase activity is a crucial step on the path to demonstrating that this class of synthetic genetic materials is in fact functional and evolvable.

### References

1. V. S. Lelyveld, W. Zhang, J. W. Szostak, Synthesis of phosphoramidate-linked DNA by a modified DNA polymerase. Proceedings of the National Academy of Sciences. 117, 7276-7283 (2020).
2. F. Eckstein, Nucleoside Phosphorothioates. Annual Review of Biochemistry. 54, 367-402 (1985).
3. P. Li, Z. A. Sergueeva, M. Dobrikov, B. R. Shaw, Nucleoside and Oligonucleoside Boranophosphates: Chemistry and Properties. Chem. Rev. 107, 4746-4796 (2007).
4. S. Arangundy-Franklin, A. I. Taylor, B. T. Porebski, V. Genna. S. Peak-Chew, A. Vaisman, R. Woodgate, M. Orozco, P. Holliger, A synthetic genetic polymer with an uncharged backbone chemistry based on alkyl phosphonate nucleic acids. Nature Chemistry (2019), doi:10.1038/s41557-019-0255-4.
5. R. L. Letsinger, J. S. Wilkes, L. B. Dumas, Nucleotide chemistry. XVII. Enzymic synthesis of polydeoxyribonucleotides possessing internucleotide phosphoramidate bonds. Journal of the American Chemical Society. 94, 292-293 (1972).
6. A. K. Vashishtha, J. Wang, W. H. Konigsberg, Different Divalent Cations Alter the Kinetics and Fidelity of DNA Polymerases. Journal of Biological Chemistry. 291, 20869-20875 (2016).
7. T. A. Steitz, A mechanism for all polymerases. Nature. 391, 231-232 (1998).
8. N. Freund, M. J. L. J. Fürst, P. Holliger, New chemistries and enzymes for synthetic genetics. Current Opinion in Biotechnology. 74, 129-136 (2022).
9. T. Nakamura, Y. Zhao, Y. Yamagata, Y. Hua, W. Yang, Watching DNA polymerase η make a phosphodiester bond. Nature. 487, 196-201 (2012).
10. Y. Gao, W. Yang, Capture of a third Mg2+ is essential for catalyzing DNA synthesis. Science. 352, 1334-1337 (2016).
11. N. Chim, L. N. Jackson, A. M. Trinh, J. C. Chaput, Crystal structures of DNA polymerase I capture novel intermediates in the DNA synthesis pathway. eLife. 7, doi:10.7554/eLife.40444.
12. C. Castro, E. D. Smidansky, J. J. Arnold, K. R. Maksimchuk, I. Moustafa, A. Uchida, M. Götte, W. Konigsberg, C. E. Cameron, Nucleic acid polymerases use a general acid for nucleotidyl transfer. Nat Struct Mol Biol. 16, 212-218 (2009).
13. V. Tereshko, S. Gryaznov, M. Egli, Consequences of Replacing the DNA 3′-Oxygen by an Amino Group: High-Resolution Crystal Structure of a Fully Modified N3′→P5′ Phosphoramidate DNA Dodecamer Duplex. Journal of the American Chemical Society. 120, 269-283 (1998).
14. K. A. Johnson, The kinetic and chemical mechanism of high-fidelity DNA polymerases. Biochimica et Biophysica Acta (BBA)—Proteins and Proteomics. 1804, 1041-1048 (2010).
15. C. M. Joyce, S. J. Benkovic, DNA Polymerase Fidelity: Kinetics, Structure, and Checkpoints. Biochemistry. 43, 14317-14324 (2004).
16. C. Castro, E. Smidansky, K. R. Maksimchuk, J. J. Arnold, V. S. Korneeva, M. Gotte, W. Konigsberg, C. E. Cameron, Two proton transfers in the transition state for nucleotidyl transfer catalyzed by RNA- and DNA-dependent RNA and DNA polymerases. Proceedings of the National Academy of Sciences. 104, 4267-4272 (2007).
17. D. Herschlag, J. A. Piccirilli, T. R. Cech, Ribozyme-catalyzed and nonenzymic reactions of phosphate diesters: rate effects upon substitution of sulfur for a nonbridging phosphoryl oxygen atom. Biochemistry. 30, 4844-4854 (1991).
18. W.-J. Wu, W. Yang, M.-D. Tsai, How DNA polymerases catalyse replication and repair with contrasting fidelity. Nat Rev Chem. 1, 1-16 (2017).
19. C. A. Sucato, T. G. Upton, B. A. Kashemirov, J. Osuna, K. Oertell, W. A. Beard, S. H. Wilson, J. Florián, A. Warshel, C. E. McKenna, M. F. Goodman, DNA Polymerase β Fidelity: Halomethylene-Modified Leaving Groups in Pre-Steady-State Kinetic Analysis Reveal Differences at the Chemical Transition State. Biochemistry. 47, 870-879 (2008).
20. K. Oertell, B. T. Chamberlain, Y. Wu, E. Ferri, B. A. Kashemirov, W. A. Beard, S. H. Wilson, C. E. McKenna, M. F. Goodman, Transition State in DNA Polymerase β Catalysis: Rate-Limiting Chemistry Altered by Base-Pair Configuration. Biochemistry. 53, 1842-1848 (2014).
21. S. A. Wood, The aqueous geochemistry of the rare-earth elements: Critical stability constants for complexes with simple carboxylic acids at 25° C. and 1 bar and their application to nuclear waste management. Engineering Geology. 34, 229-259 (1993).
22. A. Osland, K. Kleppe, Influence of polyamines on the activity of DNA polymerase I from Escherichia coli. Biochimica et Biophysica Acta (BBA)—Nucleic Acids and Protein Synthesis. 520, 317-330 (1978).
23. A. I. Taylor, P. Holliger, Directed evolution of artificial enzymes (XNAzymes) from diverse repertoires of synthetic genetic polymers. Nat Protoc. 10, 1625-1642 (2015).
24. A. A. Henry, F. E. Romesberg, The evolution of DNA polymerases with novel activities. Current Opinion in Biotechnology. 16, 370-377 (2005).
25. S. Gryaznov, Oligonucleotide N3′→P5′ phosphoramidates as antisense agents. Nucleic Acids Research. 24, 1508-1514 (1996).
26. R. Lohrmann, L. E. Orgel, Template-directed synthesis of high molecular weight polynucleotide analogues. Nature. 261, 342-344 (1976).
27. W. S. Zielinski, L. E. Orgel, Oligomerization of activated derivatives of 3′-amino-3′-deoxyguanosine on poly(C) and poly(dC) templates. Nucleic Acids Research. 13, 2469-2484 (1985).
28. S. Zhang, J. C. Blain, D. Zielinska, S. M. Gryaznov, J. W. Szostak, Fast and accurate nonenzymatic copying of an RNA-like synthetic genetic polymer. Proceedings of the National Academy of Sciences. 110, 17732-17737 (2013).
29. W. Zhang, A. Pal, A. Ricardo, J. W. Szostak, Template-Directed Nonenzymatic Primer Extension Using 2-Methylimidazole-Activated Morpholino Derivatives of Guanosine and Cytidine. J. Am. Chem. Soc. 141, 12159-12166 (2019).
30. S. S. Mansy, J. P. Schrum, M. Krishnamurthy, S. Tobé, D. A. Treco, J. W. Szostak, Template-directed synthesis of a genetic polymer in a model protocell. Nature. 454, 122-125 (2008).
31. D. K. O'Flaherty, L. Zhou, J. W. Szostak, Nonenzymatic Template-Directed Synthesis of Mixed-Sequence 3′-NP-DNA up to 25 Nucleotides Long Inside Model Protocells. Journal of the American Chemical Society. 141, 10481-10488 (2019).
32. V. S. Lelyveld, D. K. O'Flaherty, L. Zhou, E. C. Izgu, J. W. Szostak, DNA polymerase activity on synthetic N3′→P5′ phosphoramidate DNA templates. Nucleic Acids Research. 47, 8941-8949 (2019).
33. S. Gryaznov, J.-K. Chen, Oligodeoxyribonucleotide N3′->P5′ Phosphoramidates: synthesis and Hybridization Properties. Journal of the American Chemical Society. 116, 3143-3144 (1994).
34. R. L. Hanna, S. M. Gryaznov, J. A. Doudna, A phosphoramidate substrate analog is a competitive inhibitor of the Tetrahymena group I ribozyme. Chemistry & Biology. 7, 845-854 (2000).
35. E. Kervio, A. Hochgesand, U. E. Steiner, C. Richert, Templating efficiency of naked DNA. Proceedings of the National Academy of Sciences. 107, 12074-12079 (2010).
36. N. L. Samara, Y. Gao, J. Wu, W. Yang, “Detection of Reaction Intermediates in Mg 2+-Dependent DNA Synthesis and RNA Degradation by Time-Resolved X-Ray Crystallography” in Methods in Enzymology (Elsevier, 2017), vol. 592, pp. 283-327.

### Other Embodiments

All of the features disclosed in this specification can be combined in any combination. Each feature disclosed in this specification can be replaced by an alternative feature serving the same, equivalent, or similar purpose. Thus, unless expressly stated otherwise, each feature disclosed is only an example of a generic series of equivalent or similar features.

Embodiment 1 is a method for producing an oligonucleotide or a polynucleotide comprising phosphoramidate-linked nucleotides, the method comprising incubating a sample comprising a DNA polymerase variant comprising an amino acid sequence that is at least 70% identical to SEQ ID NO: 1, wherein the amino acid sequence comprises an amino acid substitution at position F710, a trivalent metal ion cofactor, 3′-amino-2′,3′-dideoxyribonucleotide 5′-triphosphates (nNTPs), a 3′-amino terminated primer, and a DNA template comprising, from 3′ to 5′, a sequence complementary to the primer and a nucleic acid sequence of interest, under conditions and for a time sufficient for the DNA polymerase variant to produce the oligonucleotide or the polynucleotide comprising phosphoramidate-linked nucleotides.

Embodiment 2 is the method of embodiment 1, wherein the amino acid sequence is at least 80% identical to SEQ ID NO: 1.

Embodiment 3 is the method of embodiment 1, wherein the amino acid sequence is at least 90% identical to SEQ ID NO: 1.

Embodiment 4 is the method of embodiment 1, wherein the amino acid sequence is at least 95% identical to SEQ ID NO: 1.

Embodiment 5 is the method of embodiment 1, wherein the amino acid substitution at position F710 is F710Y.

Embodiment 6 is the method of embodiment 1, wherein the DNA polymerase variant comprises the amino acid sequence set forth in SEQ ID NO: 1.

Embodiment 7 is the method of any one of embodiments 1-6, wherein the DNA polymerase variant is prepared from cells that express the DNA polymerase variant.

Embodiment 8 is the method of any one of embodiments 1-6, wherein the DNA polymerase variant is in a cell lysate from cells that express the DNA polymerase variant.

Embodiment 9 is the method of any one of embodiments 1-8, wherein the sample is incubated at a temperature of 50 to 65° C.

Embodiment 10 is the method of embodiment 9, wherein the sample is incubated at a temperature of 55° C.

Embodiment 11 is the method of any one of embodiments 1-10, wherein the sample is incubated for 1 to 24 hours.

Embodiment 12 is the method of embodiment 11, wherein the sample is incubated for 24 hours.

Embodiment 13 is the method of any one of embodiments 1-12, wherein the sample is incubated at a pH of 7 to 10.

Embodiment 14 is the method of embodiment 13, wherein the sample is incubated at a pH of 8.6-9.2.

Embodiment 15 is the method of embodiment 13, wherein the sample is incubated at a pH of 8.8.

Embodiment 16 is the method of any one of embodiments 1-15, wherein the trivalent metal ion cofactor is selected from the group consisting of Sc³⁺, Y³⁺, Lu³⁺, In³⁺, Eu³⁺, Gd³⁺, Yb³⁺, and Er³⁺.

Embodiment 17 is the method of embodiment 16, wherein the trivalent metal ion cofactor is Sc³⁺.

Embodiment 18 is the method of any one of embodiments 1-17, wherein the nNTPs comprise nATP, nGTP, nCTP, and/or nTTP.

Embodiment 19 is the method of any one of embodiments 1-18, wherein the 3′-amino terminated primer comprises ribonucleotides and/or deoxyribonucleotides.

Embodiment 20 is the method of any one of embodiments 1-19, wherein the 3′-amino terminated primer comprises a 3′-amino terminal ribonucleotide selected from the group consisting of 3′-amino-adenosine, 3′-amino-guanosine, 3′-amino-cytidine, and 3′-amino-uridine.

Embodiment 21 is the method of any one of embodiments 1-19, wherein the 3′-amino terminated primer comprises a 3′-amino terminal dideoxynucleotide selected from the group consisting of 3′-amino-2′,3′-dideoxyadenosine (nA), 3′-amino-2′,3′-dideoxythymidine (nT), 3′-amino-2′,3′-dideoxycytidine (nC), or 3′-amino-2′,3′-dideoxyguanosine (nG).

Embodiment 22 is the method of any one of embodiments 1-21, wherein the 3′-amino terminated primer comprises a label.

Embodiment 23 is the method of any one of embodiments 1-22, wherein the 3′-amino terminated primer comprises phosphodiester-linked nucleotides and/or phosphoramidate-linked nucleotides.

Embodiment 24 is the method of any one of embodiments 1-23, wherein the 3′-amino terminated primer comprises phosphorothioate-linked nucleotides.

Embodiment 25 is the method of any one of embodiments 1-24, wherein the 3′-amino terminated primer is 5 to 200 nucleotides in length.

Embodiment 26 is the method of any one of embodiments 1-25, wherein the 3′-amino terminated primer comprises at least 5 consecutive phosphoramidate-linked nucleotides.

Embodiment 27 is the method of any one of embodiments 1-25, wherein the 3′-amino terminated primer comprises at least 25 consecutive phosphoramidate-linked nucleotides.

Embodiment 28 is the method of any one of embodiments 1-25, wherein each nucleotide in the 3′-amino terminated primer is phosphoramidate-linked.

Embodiment 29 is the method of any one of embodiments 1-28, wherein the oligonucleotide or the polynucleotide is 25 to 250 nucleotides in length.

Embodiment 30 is the method of any one of embodiments 1-29, wherein the oligonucleotide or the polynucleotide comprises phosphoramidate-linked nucleotides and phosphodiester-linked nucleotides.

Embodiment 31 is the method of any one of embodiments 1-30, wherein the oligonucleotide or the polynucleotide comprises at least 25 consecutive phosphoramidate-linked nucleotides.

Embodiment 32 is the method of any one of embodiments 1-31, wherein the oligonucleotide or the polynucleotide comprises at least 50 consecutive phosphoramidate-linked nucleotides.

Embodiment 33 is the method of any one of embodiments 1-32, wherein the oligonucleotide or the polynucleotide comprises at least 100 consecutive phosphoramidate-linked nucleotides.

Embodiment 34 is the method of any one of embodiments 1-33, wherein each nucleotide in the oligonucleotide or the polynucleotide is phosphoramidate-linked.

Embodiment 35 is the method of any one of embodiments 1-34, wherein the sample further comprises nucleoside triphosphates (NTPs).

Embodiment 36 is the method of embodiment 35, wherein the NTPs comprise deoxynucleoside triphosphates (dNTPs).

Embodiment 37 is the method of any one of embodiments 1-36, wherein the sample further comprises a metal chelating agent.

Embodiment 38 is the method of embodiment 37, wherein the metal chelating agent comprises citrate, nitrilotriacetic acid (NTA), isocitrate, malate, malonate, tripolyphosphate (TPP) sodium salt, or combinations of any of these.

Embodiment 39 is the method of embodiment 37, wherein the metal chelating agent comprises citrate.

Embodiment 40 is the method of any one of embodiments 1-39, wherein the sample further comprises a polyamine.

Embodiment 41 is the method of embodiment 40, wherein the polyamine comprises spermine, putrescine, spermidine, cadaverine, thermospermine, caldopentamine, caldohexamine, or a combination of any of these.

Embodiment 42 is the method of any one of embodiments 1-41, wherein the sample further comprises a divalent metal ion cofactor.

Embodiment 43 is the method of embodiment 42, wherein the divalent metal ion cofactor is selected from the group consisting of Ba²⁺, Sr²⁺, Ca²⁺, Mg²⁺, Mn²⁺, Co²⁺, and Zn²⁺.

Embodiment 44 is an oligonucleotide or a polynucleotide comprising a plurality of phosphoramidate linkages, wherein the oligonucleotide or the polynucleotide is 25 to 1000 nucleotides in length.

Embodiment 45 is the oligonucleotide or the polynucleotide of embodiment 44, wherein the oligonucleotide or the polynucleotide is 25 to 500 nucleotides in length.

Embodiment 46 is the oligonucleotide or the polynucleotide of embodiment 44 or embodiment 45, wherein the oligonucleotide or the polynucleotide is 25 to 250 nucleotides in length.

Embodiment 47 is the oligonucleotide or the polynucleotide of any one of embodiments 44-46, wherein the oligonucleotide or the polynucleotide comprises phosphoramidate-linked nucleotides and phosphodiester-linked nucleotides.

Embodiment 48 is the oligonucleotide or the polynucleotide of any one of embodiments 44-47, wherein the oligonucleotide or the polynucleotide comprises at least 25 consecutive phosphoramidate-linked nucleotides.

Embodiment 49 is the oligonucleotide or the polynucleotide of any one of embodiments 44-48, wherein the oligonucleotide or the polynucleotide comprises at least 50 consecutive phosphoramidate-linked nucleotides.

Embodiment 50 is the oligonucleotide or the polynucleotide of any one of embodiments 44-49, wherein the oligonucleotide or the polynucleotide comprises at least 100 consecutive phosphoramidate-linked nucleotides.

Embodiment 51 is the oligonucleotide or the polynucleotide of any one of embodiments 44-50, wherein each nucleotide in the oligonucleotide or the polynucleotide is phosphoramidate-linked.

Embodiment 52 is the oligonucleotide or the polynucleotide of any one of embodiments 44-51, wherein the oligonucleotide or the polynucleotide comprises ribonucleotides and/or deoxyribonucleotides.

Embodiment 53 is a method for producing an oligonucleotide or the polynucleotide comprising thiophosphoramidate-linked nucleotides, the method comprising incubating a sample comprising a DNA polymerase variant comprising an amino acid sequence that is at least 70% identical to SEQ ID NO: 1, wherein the amino acid sequence comprises an amino acid substitution at position F710, a trivalent metal ion cofactor, a nucleoside 5′-(α-P-thio)triphosphate, a 3′-amino terminated primer, and a DNA template comprising, from 3′ to 5′, a sequence complementary to the primer and a nucleic acid sequence of interest, under conditions and for a time sufficient for the DNA polymerase variant to produce the oligonucleotide or the polynucleotide comprising at least one thiophosphoramidate-linked nucleotide.

Embodiment 54 is the method of embodiment 53, wherein the α-P-thio substituted nucleoside comprises 2′-deoxynucleoside 5′-(α-P-thio)triphosphates (dNTPαS).

Embodiment 55 is the method of embodiment 54, wherein the dNTPαS comprises 2′-deoxyadenosine 5′-(α-P-thio)triphosphates (dATPαS), 2′-deoxycytidine 5′-(α-P-thio)triphosphates (dCTPαS), 2′-deoxyguanosine 5′-(α-P-thio)triphosphates (dGTPαS), 2′-deoxythymidine 5′-(α-P-thio)triphosphates (dTTPαS), or combinations thereof.

Embodiment 56 is the method of embodiment 53 or embodiment 54, wherein the α-P-thio substituted nucleoside is stereopure.

Embodiment 57 is the method of any one of embodiments 53-56, wherein the sample is incubated at a temperature of 40 to 50° C.

Embodiment 58 is the method of any one of embodiments 53-57, wherein the oligonucleotide or the polynucleotide is 25 to 250 nucleotides in length.

Embodiment 59 is the method of any one of embodiments 53-58, wherein the oligonucleotide or the polynucleotide comprises thiophosphoramidate-linked nucleotides, phosphodiester-linked nucleotides, phosphorothioate-linked nucleotides, phosphoramidate-linked nucleotides, or a combination of any of these.

Embodiment 60 is the method of any one of embodiments 53-59, wherein the oligonucleotide or the polynucleotide comprises at least 25 consecutive thiophosphoramidate-linked nucleotides.

Embodiment 61 is the method of any one of embodiments 53-60, wherein each nucleotide in the oligonucleotide or the polynucleotide is thiophosphoramidate-linked.

Embodiment 62 is an oligonucleotide or a polynucleotide comprising a plurality of thiophosphoramidate linkages, wherein the oligonucleotide or the polynucleotide is 25 to 1000 nucleotides in length.

Embodiment 63 is the oligonucleotide or the polynucleotide of embodiment 62, wherein the oligonucleotide or the polynucleotide is 25 to 500 nucleotides in length.

Embodiment 64 is the oligonucleotide or the polynucleotide of embodiment 62 or embodiment 63, wherein the oligonucleotide or the polynucleotide is 25 to 250 nucleotides in length.

Embodiment 65 is the oligonucleotide or the polynucleotide of any one of embodiments 62-64, wherein the oligonucleotide or the polynucleotide comprises thiophosphoramidate-linked nucleotides and phosphodiester-linked nucleotides.

Embodiment 66 is the oligonucleotide or the polynucleotide of any one of embodiments 62-65, wherein the oligonucleotide or the polynucleotide comprises at least 25 consecutive thiophosphoramidate-linked nucleotides.

Embodiment 67 is the oligonucleotide or the polynucleotide of any one of embodiments 62-66, wherein the oligonucleotide or the polynucleotide comprises at least 50 consecutive thiophosphoramidate-linked nucleotides.

Embodiment 68 is the oligonucleotide or the polynucleotide of any one of embodiments 62-67, wherein the oligonucleotide or the polynucleotide comprises at least 100 consecutive thiophosphoramidate-linked nucleotides.

Embodiment 69 is the oligonucleotide or the polynucleotide of any one of embodiments 62-68, wherein each nucleotide in the oligonucleotide or the polynucleotide is thiophosphoramidate-linked.

Embodiment 70 is the oligonucleotide or the polynucleotide of any one of embodiments 62-69, wherein at least one of the thiophosphoramidate-linked nucleotides is stereopure.

Embodiment 71 is the oligonucleotide or the polynucleotide of any one of embodiments 62-70, wherein each of the thiophosphoramidate-linked nucleotides are stereopure.

Embodiment 72 is the oligonucleotide or the polynucleotide of any one of embodiments 62-71, wherein the oligonucleotide or the polynucleotide comprises ribonucleotides and/or deoxyribonucleotides.

Embodiment 73 is a kit for producing an oligonucleotide or a polynucleotide comprising phosphoramidate-linked nucleotides or thiophosphoramidate-linked nucleotides, the kit comprising one or more containers of a DNA polymerase variant comprising an amino acid sequence that is at least 70% identical to SEQ ID NO:1, and a trivalent metal ion cofactor.

Embodiment 74 is a method for producing an oligonucleotide or the polynucleotide comprising thiophosphoramidate-linked nucleotides, the method comprising incubating a sample comprising a DNA polymerase variant comprising an amino acid sequence that is at least 70% identical to SEQ ID NO: 1, wherein the amino acid sequence comprises an amino acid substitution at position F710, a trivalent metal ion cofactor, a nucleoside 5′-(α-P-thio)triphosphate, a 3′-amino terminated primer, and a DNA template comprising, from 3′ to 5′, a sequence complementary to the primer and a nucleic acid sequence of interest, under conditions and for a time sufficient for the DNA polymerase variant to produce the oligonucleotide or the polynucleotide comprising at least one thiophosphoramidate-linked nucleotide.

Embodiment 75 is the method of embodiment 74, wherein the amino acid sequence is at least 80% identical to SEQ ID NO: 1.

Embodiment 76 is the method of embodiment 74, wherein the amino acid sequence is at least 90% identical to SEQ ID NO: 1.

Embodiment 77 is the method of embodiment 74, wherein the amino acid sequence is at least 95% identical to SEQ ID NO: 1.

Embodiment 78 is the method of embodiment 74, wherein the amino acid substitution at position F710 is F710Y.

Embodiment 79 is the method of embodiment 74, wherein the DNA polymerase variant comprises the amino acid sequence set forth in SEQ ID NO: 1.

Embodiment 80 is the method of any one of embodiments 74-79, wherein the DNA polymerase variant is prepared from cells that express the DNA polymerase variant.

Embodiment 81 is the method of any one of embodiments 74-79, wherein the DNA polymerase variant is in a cell lysate from cells that express the DNA polymerase variant.

Embodiment 82 is the method of any one of embodiments 74-81, wherein the sample is incubated at a temperature of 40 to 50° C.

Embodiment 83 is the method of embodiment 82, wherein the sample is incubated at a temperature of 55° C.

Embodiment 84 is the method of any one of embodiments 74-83, wherein the sample is incubated for 1 to 24 hours.

Embodiment 85 is the method of embodiment 84, wherein the sample is incubated for 24 hours.

Embodiment 86 is the method of any one of embodiments 74-85, wherein the sample is incubated at a pH of 7 to 10.

Embodiment 87 is the method of embodiment 86, wherein the sample is incubated at a pH of 8.6-9.2.

Embodiment 88 is the method of embodiment 86, wherein the sample is incubated at a pH of 8.8.

Embodiment 89 is the method of any one of embodiments 74-88, wherein the trivalent metal ion cofactor is selected from the group consisting of Sc³⁺, Y³⁺, Lu³⁺, In³⁺, Eu³⁺, Gd³⁺, Yb³⁺, and Er³⁺.

Embodiment 90 is the method of embodiment 89, wherein the trivalent metal ion cofactor is Sc³⁺.

Embodiment 91 is the method of any one of embodiments 74-89, wherein the α-P-thio substituted nucleoside comprises 2′-deoxynucleoside 5′-(α-P-thio)triphosphates (dNTPαS).

Embodiment 92 is the method of embodiment 91, wherein the dNTPαS comprises 2′-deoxyadenosine 5′-(α-P-thio)triphosphates (dATPαS), 2′-deoxycytidine 5′-(α-P-thio)triphosphates (dCTPαS), 2′-deoxyguanosine 5′-(α-P-thio)triphosphates (dGTPαS), 2′-deoxythymidine 5′-(α-P-thio)triphosphates (dTTPαS), or combinations thereof.

Embodiment 93 is the method of embodiment 91 or embodiment 92, wherein the α-P-thio substituted nucleoside is stereopure.

Embodiment 94 is the method of any one of embodiments 74-93, wherein the 3′-amino terminated primer comprises ribonucleotides and/or deoxyribonucleotides.

Embodiment 95 is the method of any one of embodiments 74-94, wherein the 3′-amino terminated primer comprises a 3′-amino terminal ribonucleotide selected from the group consisting of 3′-amino-adenosine, 3′-amino-guanosine, 3′-amino-cytidine, and 3′-amino-uridine.

Embodiment 96 is the method of any one of embodiments 74-94, wherein the 3′-amino terminated primer comprises a 3′-amino terminal dideoxynucleotide selected from the group consisting of 3′-amino-2′,3′-dideoxyadenosine (nA), 3′-amino-2′,3′-dideoxythymidine (nT), 3′-amino-2′,3′-dideoxycytidine (nC), or 3′-amino-2′,3′-dideoxyguanosine (nG).

Embodiment 97 is the method of any one of embodiments 74-96, wherein the 3′-amino terminated primer comprises a label.

Embodiment 98 is the method of any one of embodiments 74-97, wherein the 3′-amino terminated primer comprises phosphodiester-linked nucleotides and/or phosphoramidate-linked nucleotides.

Embodiment 99 is the method of any one of embodiments 74-98, wherein the 3′-amino terminated primer comprises phosphorothioate-linked nucleotides.

Embodiment 100 is the method of any one of embodiments 74-99, wherein the 3′-amino terminated primer is 5 to 200 nucleotides in length.

Embodiment 101 is the method of any one of embodiments 74-100, wherein the 3′-amino terminated primer comprises at least 5 consecutive phosphoramidate-linked nucleotides.

Embodiment 102 is the method of any one of embodiments 74-100, wherein the 3′-amino terminated primer comprises at least 25 consecutive phosphoramidate-linked nucleotides.

Embodiment 103 is the method of any one of embodiments 74-100, wherein each nucleotide in the 3′-amino terminated primer is phosphoramidate-linked.

Embodiment 104 is the method of any one of embodiments 74-103, wherein the oligonucleotide or the polynucleotide is 25 to 250 nucleotides in length.

Embodiment 105 is the method of any one of embodiments 74-104, wherein the oligonucleotide or the polynucleotide comprises thiophosphoramidate-linked nucleotides, phosphodiester-linked nucleotides, phosphorothioate-linked nucleotides, phosphoramidate-linked nucleotides, or a combination of any of these.

Embodiment 106 is the method of any one of embodiments 74-105, wherein the oligonucleotide or the polynucleotide comprises at least 25 consecutive thiophosphoramidate-linked nucleotides.

Embodiment 107 is the method of any one of embodiments 74-106, wherein each nucleotide in the oligonucleotide or the polynucleotide is thiophosphoramidate-linked.

Embodiment 108 is the method of any one of embodiments 74-107, wherein the sample further comprises nucleoside triphosphates (NTPs).

Embodiment 109 is the method of embodiment 108, wherein the NTPs comprise deoxynucleoside triphosphates (dNTPs).

Embodiment 110 is the method of any one of embodiments 74-109, wherein the sample further comprises a metal chelating agent.

Embodiment 111 is the method of embodiment 110, wherein the metal chelating agent comprises citrate, nitrilotriacetic acid (NTA), isocitrate, malate, malonate, tripolyphosphate (TPP) sodium salt, or combinations of any of these.

Embodiment 112 is the method of embodiment 110, wherein the metal chelating agent comprises citrate.

Embodiment 113 is the method of any one of embodiments 74-112, wherein the sample further comprises a polyamine.

Embodiment 114 is the method of embodiment 113, wherein the polyamine comprises spermine, putrescine, spermidine, cadaverine, thermospermine, caldopentamine, caldohexamine, or a combination of any of these.

Embodiment 115 is the method of any one of embodiments 74-114, wherein the sample further comprises a divalent metal ion cofactor.

Embodiment 116 is the method of embodiment 115, wherein the divalent metal ion cofactor is selected from the group consisting of Ba²⁺, Sr²⁺, Ca²⁺, Mg²⁺, Mn²⁺, Co²⁺, and Zn²⁺.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments can be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases can encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for producing an oligonucleotide or a polynucleotide comprising phosphoramidate-linked nucleotides, the method comprising:
incubating a sample comprising a DNA polymerase variant comprising an amino acid sequence that is at least 70% identical to SEQ ID NO: 1, wherein the amino acid sequence comprises an amino acid substitution at position F710, a trivalent metal ion cofactor, 3′-amino-2′,3′-dideoxyribonucleotide 5′-triphosphates (nNTPs), a 3′-amino terminated primer, and a DNA template comprising, from 3′ to 5′, a sequence complementary to the primer and a nucleic acid sequence of interest, under conditions and for a time sufficient for the DNA polymerase variant to produce the oligonucleotide or the polynucleotide comprising phosphoramidate-linked nucleotides.

2. The method of claim 1, wherein the amino acid sequence is at least 80% identical to SEQ ID NO: 1.

3. The method of claim 1, wherein the amino acid sequence is at least 90% identical to SEQ ID NO: 1.

4. The method of claim 1, wherein the amino acid sequence is at least 95% identical to SEQ ID NO: 1.

5. The method of claim 1, wherein the amino acid substitution at position F710 is F710Y.

6. The method of claim 1, wherein the DNA polymerase variant comprises the amino acid sequence set forth in SEQ ID NO: 1.

7. The method of any one of claims 1-6, wherein the DNA polymerase variant is prepared from cells that express the DNA polymerase variant.

8. The method of any one of claims 1-6, wherein the DNA polymerase variant is in a cell lysate from cells that express the DNA polymerase variant.

9. The method of any one of claims 1-8, wherein the sample is incubated at a temperature of 50 to 65° C.

10. The method of claim 9, wherein the sample is incubated at a temperature of 55° C.

11. The method of any one of claims 1-10, wherein the sample is incubated for 1 to 24 hours.

12. The method of claim 11, wherein the sample is incubated for 24 hours.

13. The method of any one of claims 1-12, wherein the sample is incubated at a pH of 7 to 10.

14. The method of claim 13, wherein the sample is incubated at a pH of 8.6-9.2.

15. The method of claim 13, wherein the sample is incubated at a pH of 8.8.

16. The method of any one of claims 1-15, wherein the trivalent metal ion cofactor is selected from the group consisting of Sc³⁺, Y³⁺, Lu³⁺, In³⁺, Eu³⁺, Gd³⁺, Yb³⁺, and Er³⁺.

17. The method of claim 16, wherein the trivalent metal ion cofactor is Sc³⁺.

18. The method of any one of claims 1-17, wherein the nNTPs comprise nATP, nGTP, nCTP, and/or nTTP.

19. The method of any one of claims 1-18, wherein the 3′-amino terminated primer comprises ribonucleotides and/or deoxyribonucleotides.

20. The method of any one of claims 1-19, wherein the 3′-amino terminated primer comprises a 3′-amino terminal ribonucleotide selected from the group consisting of 3′-amino-adenosine, 3′-amino-guanosine, 3′-amino-cytidine, and 3′-amino-uridine.

21. The method of any one of claims 1-19, wherein the 3′-amino terminated primer comprises a 3′-amino terminal dideoxynucleotide selected from the group consisting of 3′-amino-2′,3′-dideoxyadenosine (nA), 3′-amino-2′,3′-dideoxythymidine (nT), 3′-amino-2′,3′-dideoxycytidine (nC), or 3′-amino-2′,3′-dideoxyguanosine (nG).

22. The method of any one of claims 1-21, wherein the 3′-amino terminated primer comprises a label.

23. The method of any one of claims 1-22, wherein the 3′-amino terminated primer comprises phosphodiester-linked nucleotides and/or phosphoramidate-linked nucleotides.

24. The method of any one of claims 1-23, wherein the 3′-amino terminated primer comprises phosphorothioate-linked nucleotides.

25. The method of any one of claims 1-24, wherein the 3′-amino terminated primer is 5 to 200 nucleotides in length.

26. The method of any one of claims 1-25, wherein the 3′-amino terminated primer comprises at least 5 consecutive phosphoramidate-linked nucleotides.

27. The method of any one of claims 1-25, wherein the 3′-amino terminated primer comprises at least 25 consecutive phosphoramidate-linked nucleotides.

28. The method of any one of claims 1-25, wherein each nucleotide in the 3′-amino terminated primer is phosphoramidate-linked.

29. The method of any one of claims 1-28, wherein the oligonucleotide or the polynucleotide is 25 to 250 nucleotides in length.

30. The method of any one of claims 1-29, wherein the oligonucleotide or the polynucleotide comprises phosphoramidate-linked nucleotides and phosphodiester-linked nucleotides.

31. The method of any one of claims 1-30, wherein the oligonucleotide or the polynucleotide comprises at least 25 consecutive phosphoramidate-linked nucleotides.

32. The method of any one of claims 1-31, wherein the oligonucleotide or the polynucleotide comprises at least 50 consecutive phosphoramidate-linked nucleotides.

33. The method of any one of claims 1-32, wherein the oligonucleotide or the polynucleotide comprises at least 100 consecutive phosphoramidate-linked nucleotides.

34. The method of any one of claims 1-33, wherein each nucleotide in the oligonucleotide or the polynucleotide is phosphoramidate-linked.

35. The method of any one of claims 1-34, wherein the sample further comprises nucleoside triphosphates (NTPs).

36. The method of claim 35, wherein the NTPs comprise deoxynucleoside triphosphates (dNTPs).

37. The method of any one of claims 1-36, wherein the sample further comprises a metal chelating agent.

38. The method of claim 37, wherein the metal chelating agent comprises citrate, nitrilotriacetic acid (NTA), isocitrate, malate, malonate, tripolyphosphate (TPP) sodium salt, or combinations of any of these.

39. The method of claim 37, wherein the metal chelating agent comprises citrate.

40. The method of any one of claims 1-39, wherein the sample further comprises a polyamine.

41. The method of claim 40, wherein the polyamine comprises spermine, putrescine, spermidine, cadaverine, thermospermine, caldopentamine, caldohexamine, or a combination of any of these.

42. The method of any one of claims 1-41, wherein the sample further comprises a divalent metal ion cofactor.

43. The method of claim 42, wherein the divalent metal ion cofactor is selected from the group consisting of Ba²⁺, Sr²⁺, Ca²⁺, Mg²⁺, Mn²⁺, Co²⁺, and Zn²⁺.

44. An oligonucleotide or a polynucleotide comprising a plurality of phosphoramidate linkages, wherein the oligonucleotide or the polynucleotide is 25 to 1000 nucleotides in length.

45. The oligonucleotide or the polynucleotide of claim 44, wherein the oligonucleotide or the polynucleotide is 25 to 500 nucleotides in length.

46. The oligonucleotide or the polynucleotide of claim 44 or claim 45, wherein the oligonucleotide or the polynucleotide is 25 to 250 nucleotides in length.

47. The oligonucleotide or the polynucleotide of any one of claims 44-46, wherein the oligonucleotide or the polynucleotide comprises phosphoramidate-linked nucleotides and phosphodiester-linked nucleotides.

48. The oligonucleotide or the polynucleotide of any one of claims 44-47, wherein the oligonucleotide or the polynucleotide comprises at least 25 consecutive phosphoramidate-linked nucleotides.

49. The oligonucleotide or the polynucleotide of any one of claims 44-48, wherein the oligonucleotide or the polynucleotide comprises at least 50 consecutive phosphoramidate-linked nucleotides.

50. The oligonucleotide or the polynucleotide of any one of claims 44-49, wherein the oligonucleotide or the polynucleotide comprises at least 100 consecutive phosphoramidate-linked nucleotides.

51. The oligonucleotide or the polynucleotide of any one of claims 44-50, wherein each nucleotide in the oligonucleotide or the polynucleotide is phosphoramidate-linked.

52. The method of any one of claims 44-51, wherein the oligonucleotide or the polynucleotide comprises ribonucleotides and/or deoxyribonucleotides.

53. A method for producing an oligonucleotide or the polynucleotide comprising thiophosphoramidate-linked nucleotides, the method comprising:
incubating a sample comprising a DNA polymerase variant comprising an amino acid sequence that is at least 70% identical to SEQ ID NO: 1, wherein the amino acid sequence comprises an amino acid substitution at position F710, a trivalent metal ion cofactor, a nucleoside 5′-(α-P-thio)triphosphate, a 3′-amino terminated primer, and a DNA template comprising, from 3′ to 5′, a sequence complementary to the primer and a nucleic acid sequence of interest, under conditions and for a time sufficient for the DNA polymerase variant to produce the oligonucleotide or the polynucleotide comprising at least one thiophosphoramidate-linked nucleotide.

54. The method of claim 53, wherein the α-P-thio substituted nucleoside comprises 2′-deoxynucleoside 5′-(α-P-thio)triphosphates (dNTPαS).

55. The method of claim 54, wherein the dNTPαS comprises 2′-deoxyadenosine 5′-(α-P-thio)triphosphates (dATPαS), 2′-deoxycytidine 5′-(α-P-thio)triphosphates (dCTPαS), 2′-deoxyguanosine 5′-(α-P-thio)triphosphates (dGTPαS), 2′-deoxythymidine 5′-(α-P-thio)triphosphates (dTTPαS), or combinations thereof.

56. The method of claim 53 or claim 54, wherein the α-P-thio substituted nucleoside is stereopure.

57. The method of any one of claims 53-56, wherein the sample is incubated at a temperature of 40 to 50° C.

58. The method of any one of claims 53-57, wherein the oligonucleotide or the polynucleotide is 25 to 250 nucleotides in length.

59. The method of any one of claims 53-58, wherein the oligonucleotide or the polynucleotide comprises thiophosphoramidate-linked nucleotides, phosphodiester-linked nucleotides, phosphorothioate-linked nucleotides, phosphoramidate-linked nucleotides, or a combination of any of these.

60. The method of any one of claims 53-59, wherein the oligonucleotide or the polynucleotide comprises at least 25 consecutive thiophosphoramidate-linked nucleotides.

61. The method of any one of claims 53-60, wherein each nucleotide in the oligonucleotide or the polynucleotide is thiophosphoramidate-linked.

62. An oligonucleotide or a polynucleotide comprising a plurality of thiophosphoramidate linkages, wherein the oligonucleotide or the polynucleotide is 25 to 1000 nucleotides in length.

63. The oligonucleotide or the polynucleotide of claim 62, wherein the oligonucleotide or the polynucleotide is 25 to 500 nucleotides in length.

64. The oligonucleotide or the polynucleotide of claim 62 or claim 63, wherein the oligonucleotide or the polynucleotide is 25 to 250 nucleotides in length.

65. The oligonucleotide or the polynucleotide of any one of claims 62-64, wherein the oligonucleotide or the polynucleotide comprises thiophosphoramidate-linked nucleotides and phosphodiester-linked nucleotides.

66. The oligonucleotide or the polynucleotide of any one of claims 62-65, wherein the oligonucleotide or the polynucleotide comprises at least 25 consecutive thiophosphoramidate-linked nucleotides.

67. The oligonucleotide or the polynucleotide of any one of claims 62-66, wherein the oligonucleotide or the polynucleotide comprises at least 50 consecutive thiophosphoramidate-linked nucleotides.

68. The oligonucleotide or the polynucleotide of any one of claims 62-67, wherein the oligonucleotide or the polynucleotide comprises at least 100 consecutive thiophosphoramidate-linked nucleotides.

69. The oligonucleotide or the polynucleotide of any one of claims 62-68, wherein each nucleotide in the oligonucleotide or the polynucleotide is thiophosphoramidate-linked.

70. The oligonucleotide or the polynucleotide of any one of claims 62-69, wherein at least one of the thiophosphoramidate-linked nucleotides is stereopure.

71. The oligonucleotide or the polynucleotide of any one of claims 62-70, wherein each of the thiophosphoramidate-linked nucleotides are stereopure.

72. The method of any one of claims 62-71, wherein the oligonucleotide or the polynucleotide comprises ribonucleotides and/or deoxyribonucleotides.

73. A kit for producing an oligonucleotide or a polynucleotide comprising phosphoramidate-linked nucleotides or thiophosphoramidate-linked nucleotides, the kit comprising:
one or more containers of a DNA polymerase variant comprising an amino acid sequence that is at least 70% identical to SEQ ID NO:1, and
a trivalent metal ion cofactor.


SEQUENCE LISTING (11 sequences)

SEQ ID NO: 1 - protein, 579 aa, Geobacillus stearothermophilus
KMAFTLADRVTEEMLADKAALVVEVVEENYHDAPIVGIAVVNEHGRFFLRPETALADPQFVAWLGDETKKKSMFDSKRAAVALKWKGIELCGVSFDLLLAAYLLDPAQGVDDVAAAAKMKQYEAVRPDEAVYGKGAKRAVPDEPVLAEHLVRKAAAIWELERPFLDELRRNEQDRLLVELEQPLSSILAEMEFAGVKVDTKRLEQMGKELAEQLGTVEQRIYELAGQEFNINSPKQLGVILFEKLQLPVLKKTKTGYSTSADVLEKLAPYHEIVENILHYRQLGKLQSTYIEGLLKVVRPDTKKVHTIFNQALTQTGRLSSTEPNLQNIPIRLEEGRKIRQAFVPSESDWLIFAADYSQIELRVLAHIAEDDNLMEAFRRDLDIHTKTAMDIFQVSEDEVTPNMRRQAKAVNFGIVYGISDYGLAQNLNISRKEAAEFIERYFESFPGVKRYMENIVQEAKQKGYVTTLLHRRRYLPDITSRNFNVRSFAERMAMNTPIQGSAADIIKKAMIDLNARLKEERLQAHLLLQVHDELILEAPKEEMERLCRLVPEVMEQAVTLRVPLKVDYHYGSTWYDAK

SEQ ID NO: 2 - skipped

SEQ ID NO: 3 - DNA, 14 bp, synthetic construct
  source 1..14: mol_type = other DNA
acacgctgatcgca

SEQ ID NO: 4 - DNA, 18 bp, synthetic construct
  source 1..18: mol_type = other DNA
gcgtccattcgttacctg

SEQ ID NO: 5 - DNA, 23 bp, synthetic construct
  source 1..23: mol_type = other DNA
cgcaggtaagcaatggacggact

SEQ ID NO: 6 - DNA, 46 bp, synthetic construct
  source 1..46: mol_type = other DNA
cgcaggtaagcaatggacggactacgcactcataagttgtaaaggc

SEQ ID NO: 7 - DNA, 23 bp, synthetic construct
  source 1..23: mol_type = other DNA
tcaggcaggtaacgaatggacgc

SEQ ID NO: 8 - DNA, 29 bp, synthetic construct
  source 1..29: mol_type = other DNA
cacgcatcaggcaggtaacgaatggacgc

SEQ ID NO: 9 - DNA, 46 bp, synthetic construct
  source 1..46: mol_type = other DNA
cggaaatgttgaatactcacgcatcaggcaggtaacgaatggacgc

SEQ ID NO: 10 - DNA, 89 bp, synthetic construct
  source 1..89: mol_type = other DNA
ttaggtgatacccgccgcgttaacgtattcgtgcagaaccatatggtcacgcatggtgaactccttcaaaacaggtaacgaatggacgc

SEQ ID NO: 11 - DNA, 118 bp, synthetic construct
  source 1..118: mol_type = other DNA
catgttgaatactcacgcannnnnnnnnnttaggtgatacccgccgcgttaacgtattcgtgcagaaccatatggtcacgcatggtgaactccttcaaaacaggtaacgaatggacgc